(19) United States
(12) Patent Application Publication
(10) Pub. No.: US 2022/0305401 A1
(43) Pub. Date: Sep. 29, 2022

(54) EUTECTIC EXTRACTION OF SOLIDS

(71) Applicant: Givaudan SA, VERNIER (CH)

(72) Inventors: Michael LAGUERRE, Le Pontet (FR); Antoine Charles BILY, Vedène (FR); Simona BIRTIC, Cavaillon (FR); Céline GERIN, ORGON (FR); Alexis LAVAUD, Muespach (FR); Mathieu TENON, Malemort du Comtat (FR)

(21) Appl. No.: 17/619,014

(22) PCT Filed: Jun. 19, 2020
(86) PCT No.: PCT/EP2020/067104

§ 371 Date: Dec. 14, 2021

(30) Foreign Application Priority Data
Jun. 20, 2019 (GB) 1908878.0

(51) Int. Cl. B01D 11/02; A23L 33/105; B01D 9/00; B01D 21/26; B01D 21/00; A23K 10/30; A23L 27/10; A23L 27/12; A61K 36/53; C11B 9/02

(52) U.S. Cl. CPC B01D 11/0288; A23L 33/105; B01D 9/0054; B01D 21/262; B01D 21/0012; A23K 10/30; A23L 27/11; A23L 27/13; A61K 36/53; C11B 9/025; A23V 2002/00; A61K 2236/333

(57) ABSTRACT

The present in relates to methods and uses for preparing biological extracts using Deep Eutectic Solvents (DES) as hydrotropic agents, methods for purifying biological extracts formed using Deep Eutectic Solvents (DES) as hydrotropic agents, the biological extractions obtained using the methods and uses and the use of the biological extracts, such as in food-stuffs, flavours and fragrances, pharmaceuticals, cosmetics, nutraceuticals and supplements, such as food supplements and sports supplements.

The present invention relates to methods and uses for preparing biological extracts using Deep Eutectic Solvents (DES) as hydrotropic agents, methods for purifying biological extracts formed using Deep Eutectic Solvents (DES) as hydrotropic agents, the biological extractions obtained using the methods and uses and the use of the biological extracts, such as in food-stuffs, flavours and fragrances, pharmaceuticals, cosmetics, nutraceuticals and supplements, such as food supplements and sports supplements.

Natural molecules, such as that present in plant materials, can be extracted, solubilized and/or stabilized by a wide variety of liquid organic solvents such as alcohols, acetone or hexane with the inherent problem, however, that these solvents are mostly petroleum-sourced and that they emit volatile organic compounds that are harmful for the environment.

Supercritical fluids, mostly carbon dioxide, can also be used for this purpose even though they are often not cost-effective at an industrial level.

Water is a natural solvent that is generally considered to be renewable. However, its high polarity drastically limits the profile of the extracted molecules. Typically, it does not easily allow for the extraction of lipophilic/non-water-soluble molecules.

Therefore, there is a need to improve existing extraction methods to prepare biological extracts with improved quality and with different proportions of lipophilic/non-water-soluble active compounds, as well as reducing the environmental impact of such methods by reducing the emission of volatile organic compounds.

Hydrotropic agents are water-soluble organic compounds which, at and above a certain concentration, known as “MHC” (Minimum Hydrotropic Concentration), provide a significant increase in the solubility of organic compounds that are practically insoluble in water under normal conditions.

The minimum hydrotropic concentration (MHC) is the concentration on and after that hydrotropes start to form aggregates i.e. new micro-environments with physical properties differing from those observed when the compound is diluted and differing from hydrotropic behaviour.

The MHC can be determined using several physicochemical methods such as measurement of surface tension, conductivity, dynamic and static light scattering (RE, Coffman, DO, Kildsig, Self-association of nicotinamide in aqueous solution: Light-scattering and vapor pressure osmometry studies (1996) 85(8): 848-853) or by plotting a solubilisation curve of a lipophilic compound (content of solubilised solute vs. hydrotrope concentration). Sudan red or disperse red 13, two lipophilic dyes easy assayed by spectrophotometry, can be used as reference.

The term ‘hydrotropy’ was coined by Carl A. Neu Berg Neuberg (C. Neuberg, Hydrotropic phenomena, Biochem Z. 76 (1916) 107) to describe the ability of certain amphiphilic molecular structures, called ‘hydrotropes’ or ‘hydrotropic agents’, to increase, when present in water, the aqueous solubility of poorly soluble compounds.

Hydrotropes are typically amphiphilic and may be ionic (anionic, cationic, zwitterionic) or non-ionic (resorcinol, nicotinamide, alkyl polyglycosides etc.) and may have various structures e.g. aromatic, aliphatic, or cyclic.

They do not form colloids such as micelles, but it is thought that they improve solubility by forming non-covalent interactions with the solute (V. Kumar, C. Raja, C. Jayakumar. A review on solubility enhancement using hydrotropic phenomena, Int. J. Pharm. Pharm, Sci. 6 (2014) 1-7).

It is believed that a hydrotropic molecule interacts with molecules that are poorly soluble in aqueous solutions via van der Waals interactions such as π-π or attractive dipole-dipole interaction (M. Neumann, C. Schmitt, K. Prieto, et al. The photo physical determination of the minimum hydrotrope concentration of aromatic hydrotropes. J. Colloid. Interface. Sci. 315 (2007) 810-813).

Hydrotropes typically contain both hydrophobic and hydrophilic fractions in them. In comparison to surfactant, they contain only a very small hydrophobic fraction N. Kapadiya, I. Singhvi, K. Mehta, K. Gauri, D. Sen, Hydrotropy: a promising tool for solubility enhancement: a review, Int. J. Drug Dev. Res. 3 (2011) 26-33).

In recent years, new classes of solvents formed by the association of at least two components have emerged as alternatives to conventional solvents (Welton T. Room-temperature ionic liquids. Solvents for synthesis and catalysis. Chem. Rev. 1999, 99, 2071-2084; Smith E L, A P Abbott, K S Ryder. Deep Eutectic Solvents (DESs) and their applications. Chem. Rev. 2014, 114, 11060-11082). These new solvents are known as Deep Eutectic Solvents (DES).

DES are mixtures of compounds having melting points much lower than those of their constituents taken in isolation. They take their name from the Greek term “eutektis” meaning “easily melted”, a term which was used for the first time by the English physician Guthrie in 1884. This phenomenon described by Abbott et al, in EP1324979 of lowering of melting points by the formation of a eutectic mixture is attributable to the establishment of inter-molecular hydrogen bonds, which have the effect of increasing the volume of the space between the chemical species thereby increasing their mobility such that they are rendered iquid or less viscous.

However, one common drawback of DESs is their relatively high viscosity which hampers their use as extraction and formulation solvents or reaction medium for synthesis. Indeed, their high viscosity not only hinders the mass transport of chemical species but also leads to handling difficulties (e.g. in filtration, decantation, and dissolution). Furthermore, due to the fact that their degradation point is generally reached before their boiling point (if any), their removal from the extract or the formulation is not possible with cost-effective techniques such as distillation or vacuum evaporation.

The listing or discussion of an apparently prior-published document in this specification should not necessarily be taken as an acknowledgement that the document is part of the state of the art or common general knowledge.

Method of Providing A Biological Extract

The present inventors have surprisingly and unexpectedly found that solid biological extracts comprising lipophilic, hydrophobic, oil soluble and/or non-water-soluble compounds can be obtained by combining/mixing biological material (such as plant biological material, algal biological material, animal biological material and/or prokaryotic biological material) with an extraction solution comprising water and a DES.

The present invention provides a method for providing a solid biological extract comprising:

i) mixing biological material (such as plant biological material, algal biological material, animal biological material and/or prokaryotic biological material) with an extraction solution comprising water and a DES;

ii) removing any undissolved biological material from the solution obtained in (i);

iii) obtaining a flocculate and/or precipitate by adding water to and/or cooling the solution obtained in step (ii);

iv) collecting the resulting solid material obtained in step (iii) from the solution; and

v) optionally drying the solid material obtained in step (iv).

This method is hereinafter referred to as the method of the invention.

In a particular embodiment of the method of the invention, in step i) the extraction solution comprises a DES and less than about 0.5% of water, less than about 0.1. less than about 0.01, 0.001, 0.0001 or less than about 0.00001% water. In a particular embodiment of the method of the invention, in step i) the extraction solution comprising a DES is free of water.

Thus in a particular embodiment it is provided a method for providing a solid biological extract composes:

i) mixing biological material (such as plant biological material, algal biological material, animal biological material and/or prokaryotic biological material) with an extraction solution comprising a DES;

ii) removing any undissolved biological material from the solution obtained in (i);

iii) obtaining a flocculate and/or precipitate by adding water to and/or cooling the solution obtained in step (ii);

iv) collecting the resulting solid material obtained m step (iii) from the solution; and

v) optionally drying the solid material obtained step (iv).

wherein the extraction solution comprising a DES is free of water.

The present invention also provides the use of an extraction solution comprising water and a DES to provide a solid biological extract from biological material (such as plant biological material, algal biological material, animal biological material and/or prokaryotic biological material), wherein the use comprises:

i) mixing biological material (such as plant biological material, algal biological material, animal biological material and/or prokaryotic biological material) with an extraction solution comprising water and a DES;

ii) removing any undissolved biological material from the solution obtained in (i);

iii) obtaining a flocculate and/or precipitate by adding water to and/or cooling the solution obtained in step (ii);

iv) collecting the resulting solid material obtained in stop (iii) from the solution; and

(v) optionally drying the solid material obtained in step (iv).

This use is hereinafter referred to as the use of the invention,

In a particular embodiment of the use of the invention, in step i) the extraction solution comprises a DES and less than about 0.5% of water, less than about 0.1, less than about 0.01, 0.001, 0.0001 or less than about 0.00001% of water.

In a particular embodiment of the use of the invention, in step i) the extraction solution comprising a DES is free of water.

In the methods and uses described herein, the extraction solvent may comprise, consist or consist essentially of water and a DES.

In a particular embodiment of the method and uses of the invention, in step i) the extraction solution comprises a DES and less than about 0.5% of water, less than about 0.1% of water, less than about 0.01, 0.001, 0.0001 or less than about 0.00001% of water.

Optionally the extraction solvent or solution may comprise, consist or consist essentially of a DES and sad solution is free of water, i.e. is substantially free of water. As used herein, the term “substantially free” means that the extract being described may contain small concentrations of water (for example up to 0.1% or 0.01% or 0.001 or 0.0001% % of water by weight of the extract).

Where the biological extract is obtained using a method or use described herein comprising steps (i) to (v) the biological extract may be considered to be, for example, a crude solid or semi-solid biological extract.

As used herein, the term “solid biological extract” is intended to mean that at least 50% by weight, such as at least 75% by weight or at least 90% or 99% by weight of the biological extract is in the form of matter that retains its shape and density when not confined

As used herein, the term “extraction solution or solvent comprising water and a DES” is intended to mean an aqueous solution containing a DES at a concentration equal to or greater than the DES's minimum hydrotropic concentration (MHC).

As used herein the term “extraction solvent or solution comprising a DES is free of water” is intended to mean an solution containing a DES at a concentration equal to or greater than the DES's minimum hydrotropic concentration (MHC) wherein the solution is substantially tree of water.

As used herein, the term “minimum hydrotropic concentration (MHC)” is the critical concentration of the hydrotrope beyond which the hydrotrope starts to solubilise compounds that are lipophilic, hydrophobic, oil soluble and/or non-water-soluble. The MHC can be determined using several physicochemical methods such as measurement of surface tension, conductivity, dynamic and static light scattering (RE, Coffman, DO Kildsig, Self-association or nicotinamide in aqueous solution: Light-scattering and vapor pressure osmometry studies (1996) 85(8): 848-853) or by plotting a solubilisation curve of a lipophilic compound (content of solubilised solute vs. hydrotrope concentration). Disperse red 13, a lipophilic dye easily assayed by spectrophotometry, can be used as reference.

As used herein, the term “plant biological material” is material that has been obtained from or is obtainable from plants, such as from plant roots and/or the aerial parts of the plant, such as leaves, flowers, stems, barks, fruits or seeds or their tissues. For example, the plant biological material may be obtained from the leaves of the plant.

As used herein, the term “algal biological material” is material that has been obtained from or is obtainable from a macroalgal or a microalgal source, such as from seaweeds, freshwater algae or cultivated populations of single cell microalgal organisms of eukaryotic nature.

As used herein, the term “animal biological material” is material that has been obtained from or is obtainable from an animal source, such as from secretions from the glands of mammals, i.e. musk.

As used herein, the term “prokaryotic biological material” is material that has been obtained from or is obtainable from single cell organisms, such as bacteria.

As will be appreciated by the person skilled in the art, as used herein the term “obtainable from” means that the plant and/or algal and/or animal and/or prokaryotic biological material may be obtained from a plant/algae/animal/prokaryote directly or may be isolated from the plant/algae/animal/prokaryote, or may be obtained from an alternative source, for example by chemical synthesis or enzymatic production. Whereas the term “obtained” as used herein, means that the extract is directly derived from the plant/algal/animal/prokaryote source.

As used herein, the term “liquid” means a state of matter in which atoms or molecules within the liquid can move freely, while remaining in contact with one another, and will take the shape of its container. Typically, a liquid will have a viscosity from about 1 cP at 20° C. to about 10,000 cP at 20° C., such as from about 50 cP at 20 to about 5,000 cP at 20° C.

Due to the presence of a DES in the extraction solution, the biological extract obtained by the methods or uses described herein is enriched in compounds that have limited solubility in aqueous solutions, i.e. compounds that are lipophilic, hydrophobic, oil soluble and/or non-water-soluble.

This, the present invention also provides a method of providing lipophilic, hydrophobic, oil soluble and/or non-water-soluble compounds from biological material (such as plant biological material, algal biological material, animal biological material and/or prokaryotic biological material) comprising:

i) mixing biological material (such as plant biological material, algal biological material, animal biological material and/or prokaryotic biological material) with an extraction solution comprising water and a DES;

ii) removing any undissolved biological material from the solution obtained in (i);

iii) obtaining a flocculate and/or precipitate by adding water to and/or cooling the solution obtained in step (ii);

iv) collecting the resulting solid material obtained in step (iii) from the solution; and

v) optionally drying the solid material obtained in step (iv).

In a particular embodiment of the method of the invention, in step i) the extraction solution comprising a DES is free of water. In a particular embodiment of the method of the invention, in step i) the extraction solution comprises a DES and less than about 0.5% of water, and less than about 0.1, less than about 0.01, 0.001, 0.0001 or less than about 0.00001%.

The present invention also provides the use of an extraction solution comprising water and a DES to provide lipophilic, hydrophobic, oil soluble and/or non-water-soluble compounds from biological material (such as plant biological material, algal biological material, animal biological material and/or prokaryotic biological material), wherein the use comprises;

i) mixing biological material (such as plant biological material algal biological material, animal biological material and/or prokaryotic biological material) with an extraction solution comprising water and a DES;

ii) removing any undissolved biological material from the solution obtained in (i);

iii) obtaining a flocculate and/or precipitate by adding water to and/or cooling the solution obtained in step (ii);

iv) collecting the resulting solid material obtained in step (iii) from the solution; and

v) optionally drying the solid material obtained in step (iv).

In a particular embodiment, in step i) the extraction solution comprises a DES and less than about 0.5 % of water, less than about 0.1, than about 0.01, 0.001, 0.0001 or less than about 0.00001% of water.

In a particular embodiment, in step i) the extraction solution comprising a DES is free of water.

As used herein, the term “enriched” means that the biological extract comprises about 0.05% or more by weight of the extract compounds that have limited solubility in aqueous solutions, i.e. compounds that are lipophilic, hydrophobic, oil soluble and/or non-water-soluble.

For example, the crude solid or semi-solid biological extract may comprise about 2% or more, about 5 or more, about 10% or mole about 20% or more, or about 40 or more by weight of the extract of compounds that have limited solubility in aqueous solutions, i.e. compounds that are lipophilic, hydrophobic, oil soluble and/or non-water-soluble, i.e. the crude biological extract may comprise from about 2% to about 60% by weight of the extract of compounds that have limited solubility in aqueous solutions, i.e., compounds that are lipophilic, hydrophobic, oil soluble and/or non-water-soluble or from about 5% to about 40% by weight of the extract of compounds that have limited solubility in aqueous solutions, i.e. compounds that are lipophilic, hydrophobic, oil soluble and/or non-water-soluble.

As used herein, the term “hydrophobic” means that the compounds are able to dissolve in fats, oils, lipids, and non-polar solvents such as hexane or toluene. For example, at least about 90% of the compound can dissolve in fats, oils, lipids, and non-polar solvents such as hexane or toluene, or at least about 95% or at least about 99% or about 100%. As used herein, the term “non-water soluble” means the compounds have a solubility in water of less than about 1 g/L, such as less than about 0.5 g/L at 20° C. For example, a solubility in water from about 0.001 g/L to about 1 g/L or are substantially insoluble in water.

As used herein, the term “hydrophobic” means that the compounds have a very low solubility in water. For example, the compounds have a solubility in water of less than 1 g/L, such as less than 0.5 g/L at 20° C. For example, a solubility in water from about 0.001 g/L to about 1 g/L or are substantially insoluble in water.

As used herein, the term “oil soluble” means that the compounds have a high solubility in oil. For example, the compounds have a solubility in oil of from about 5 g/L or more, such as from about 10 g/L or more or from about 20 g/L or more.

Compounds that have limited solubility in aqueous solutions, i.e. compounds that are lipophilic, hydrophobic, oil soluble and/or non-water-soluble that may be present in the biological extract of the invention include, but are not limited to, are phenolic compounds including phenolic acids (such as salycilic acid, rosmarinic acid or caffeic acid), phenolic esters, phenolic diterpenes (such as carnosic acid and its derivatives), flavonoids (such as hesperidin or luteolin glucuronide), secoiridoids, curcuminoids (such as curcumin, demethoxycurcuminor bisdemethoxycurcumin and its derivatives), bixin, capsaicinoids, cannabinoids, pyranoanthocyanins, stilbenes, phenolic alcohols, phenolic lipids (such as shogaol or ubiquinol), sylimarins, alkaloids, phenylpropanoids, coumarin (such as dimethoxycoumarin), organic acids (such as malic acid or tartaric acid), terpenoids including monoterpenoids, sesquiterpenoids (such as turmerone, curcumadione, procurcumadiol or dehydrocurdione), diterpenoids (such as carnosic acid or hydroxycryptotanshinone), saponins, lignans, anthraquinone, glucosinolates, sulforaphane and isothiocyanates, triterpenoids (such as ursolic acid), sapogenins or carotenoids, and mixtures thereof, from the biological material (i.e. the plant and/or algal and/or and/or animal and/or prokaryotic biological material). For example, the biological extract may comprise one or more of the compounds listed above or mixtures thereof.

These compounds may typically be natural biological flavourings, and taste modifiers, sweeteners, fragrances, biocides, antimicrobials, proteins, enzymes, colourings, pigments, surfactants, antioxidants, chelatants, emulsifiers, texturizers, vitamins and/or bioactive compounds of nutritional, cosmetic or pharmaceutical interest

The biological material (i.e. the plant and/or algal and/or animal and/or prokaryotic biological material) used in the method of the invention may be in the form of a liquid, such as fluid from the biological material, i.e. juice from a plant or fruit. Alternatively, the biological material may be in the form of a solid, such as fresh or dried biological material, which may optionally be ground and/or mashed biological material, i.e. ground or mashed material obtained or obtainable from the biological material.

The biological material is preferably plant biological material. The plant biological material may be obtained from or obtainable from plant roots and/or plant serial parts, such as the leaves, flowers, stems, barks, peels, fruits and/or seeds, their tissues (such as the rind of the fruit) or mixtures thereof. For example, the plant biological material may be the leaves of the plant, the roots or the rind of a fruit.

The plant biological material may be obtained from or obtainable from Lamiaceae (such as basil, mint, rosemary, sage, savory, marjoram, oregano, hyssop, thyme, lavender, perilla and mixtures thereof). For example, the plant biological material may be rosemary and/or sage, The plant biological material may also be obtained from or obtainable from Citrus genus (such as C. sinensis, C. medica, C. reticulate, etc), Curcuma genus (such as Curcuma longa), Ginger (Zingiber officinale), Prunus genus (such as P. africana, P. armenicana, P. dulcis, P. avium, etc), (such as cherry flower), Gardenia jasminoides (such as gardenia fruits), Sellaginelia genus, Olea europaea (such as olive leaf), Equisetu, Crithmum (Sea fennel), Rose of Jericho, Saffron flower, Jambu flower, Lavandula genus (such as Lavandula x intermedia, Lavandula angustifolia, Lavandula latifolia), milk thistle (Silybum marianum), green tea, green coffee, clove leaves, pomegranate, rice hulis, etc.

When the biological material is plant material, such as rosemary and or sage, the biological extract obtained using the method of the invention may be enriched in carnosic acid and/or its derivatives and/or other lipophilic, hydrophobic, oil soluble and/or non-water-soluble compounds present in the biological material. For example, the biological extract may be enriched in carnosic acid, 12-methoxycarnosic acid, carnosol, rosmarinic acid, palmitic acid (16:0), stearic acid (18:0), oleic, acid (18:1), linoleic acid (18:2-n6), linolenic acid (18:3-n3), gluconio acid, malic acid, tartaric acid, salycilic acid, caffeic acid, nepitrin, ursolic acid, apigenin, luteolin glucuronide, luteolin-O-(O-acetyl) glucuronide isomers, diosmetin, hispidulin cirsimaritin, chlorophyll pigments, scutellarein, nepetin, dimethoxycoumarin, rhamnazin, rosmanol, epirosmanol, epiisorosmanol, hydroxycryptotanshinone, gingerol, epirosmanol methyl ether and its isomer, ubiquinol, para-miltioc acid rosmadial, rosmaridiphenol, O-methylcamosol, genkwanin, tetrahydrohydroxyrosmaquinone, shogaol and mixtures thereof.

When the biological material is plant material, such as turmeric, the biological extract obtained using the method of the invention may be enriched in curcumin and/or its derivatives and/or other lipophilic, hydrophobic, oil soluble and/or non-water-soluble compounds present in the biological material. For example, the biological extract may be enriched in Ar-turmerone, curcumin, dihydrocurcumin, demethoxycurcumin, dehydrodemethoxycurcumin, bisdemethoxycurcumin, dehydro bisdemethoxycurcumin, curcumadione, procurumadiol, dehydrocurdione, deoxy bisdemethoxycurcumin, deoxy dehydrobisdemethoxycurcumin, O-demethyldemethoxycurcumin and mixtures thereof.

When the biological material is plant material, the biological extract obtained using the method of the invention may be enriched in lipophilic, hydrophobic, oil soluble and/or non-water-soluble compounds present in said biological material such as Lamiaceae (such as basil, mint, rosemary, sage, savory, marjoram, oregano, hyssop, thyme, lavender, perilla and mixtures thereof), Citrus genus (such as C. sinensis, C. medica, C. reticulate, etc), Curcuma genus (such as Curcuma longa), Ginger (Zingiber officinale), Prunus genus (such as P. africana, P. armenicana, P. dulcis, P. avium, etc.), (such as cherry flower) Gardenia jasminoides (such as gardenia fruits), Sellaginella genus, Olea europaea (such as olive leaf), Equisetum, Crithmum (Sea fennel), Rose of Jericho, Saffron flower, Jambu flower, Lavandula genus (such as Lavandula x intermedia, Lavandula angustifolia, Lavandula latifolia), milk thistle (Silybum marianum), green tea, green coffee, clove leaves, pomegranate, rice hulls, etc.

For example, when the biological extract is a solid biological extract provided by a method or use as defined herein comprising steps (i) to (v), the extract may comprise at least about 2% by weight of the extract of carnosic acid, hesperidin, curcumin, curcuminoids, or bixin and/or derivatives thereof, such as at least about 5% or about 10% by weight of the dried solid or semi-solid extract of carnosic acid, hesperidin, curcumin, curcuminoids, or bixin and/or derivatives thereof.

In the method or uses defined herein, the DES may be obtained from at least two compounds selected from methylamines, organic acids, sugars, polyols. amino acids and urea. As mentioned before, in the method or used defined herein, the DES may be formulated with or without water (such as solution comprising a DES essentially free of water).

Methylamines that may be used to provide the DES used in the methods and uses described herein may be selected from N-trimethylamine, oxide (TMAO), betaine, glycerophosphocholine, camitine, homarine, choline chloride, methyl sulfonium solutes including dimethylsulfonopropionate (DMSP) and derivatives thereof, for example, their halide forms, such as betaine halides (betaine HCl).

Organic adds that may be used to provide the DES used in the methods and uses described herein may be selected from levulinic acid, lactic acid, malic acid, maleic acid, pyruvic acid, fumaric acid, succinic acid, citric acid, citraconic acid, glutaric acid, glycolic acid, acetic acid, aconitic acid, tartaric acid, ascorbic acid, malonic acid, oxalic acid, glucuronic add, neuraminic acid, sialic acid, shikimic acid, phytic acid, galacturonic acid, iduronic acid, hyaluronic acid, hydroxycitric acid, lactone derivatives and derivatives thereof.

Sugars that may be used to provide the DES used in the method and uses described herein may be selected from trehalose, glucose, sucrose, lactose, ribose, galactose, fructose, etc. and derivatives thereof.

Polyols that may be used to provide the DES used in the methods and uses described herein may be selected from glycerol, erythritol, mannitol, sorbitol, xylitol, ethylene glycol, propylene glycol, ribitol, aldonitol, propanediol, inositol, pentylene glycol, and derivatives thereof (such as o-methyl-inositol).

Amino acids that may be used to provide the DES used in the methods and uses described herein may be selected from glycine, proline, taurine, lysine, etc. and derivatives thereof (e.g. ectoine, sarcosine, theanine, dimethyglycine, etc.).

For the avoidance of doubt, the DES used DES used in the methods and uses described herein may be obtained from at least two compounds selected from methylamines, organic acids, sugars, polyols, amino acids and urea and combinations of them (i.e. one or more methylamines; one or more methylamines and one or more organic acids, etc.)

For example, the DES used in the methods and uses described herein may comprise or consist or consist essentially of betaine and urea or choline chloride and urea. In another embodiment, the DES used in the methods and uses described herein may comprise or consist or consist essentially of glycerol and betaine or glycerol and choline chloride.

In another embodiment, the DES used in the methods and uses described herein may comprise or consist or consist essentially of malic acid and chorine chloride.

In another embodiment the DES used in the methods and uses described herein may comprise or consist or consist essentially of lactic acid and betaine or levulinic acid and betaine.

In another embodiment the DES used in the methods and uses described herein may comprise or consist or consist essentially of pyruvic acid and betaine or sorbitol and betaine. In another embodiment, the DES used in the methods and uses described herein may comprise or consist or consist essentially of: urea and chlorine chloride, sorbitol and levulinic acid, proline and levulinic acid, betaine and levulinic acid, betaine and proline, betaine and glucose, proline and glucose, lysine and levulinic acid, glycerol and sorbitol, glycerol and lactic acid, glucose and levulinic acid, xylitol and levulinic acid, sorbitol and lactic acid, urea and betaine HCl, or glycerol and levulinic acid.

Where two components are present in the DES, the molar ratio of the components may range from about 4:1 to about 1:4, such as from about 2:1 to about 1:2 or about 1:1. In particular, the molar ratio of the two components may be from about 1:0.5 to about 1:4.

As mentioned before, the DES may be obtained from at least two compounds selected from methylamines, organic acids, sugars, polyos, amino acids and urea and different combinations can be made.

Where three components are present in the DES (such as an organic acid, a sugar and a polyol), the molar ratio of the components may range from about 4:1.1 to about 1:4:4, or from about 1:4:1: to about 4:1:4, or from about 1:1:4 to about 4:4:1, etc) such as from about 2:2:1 to about 1:1:2 or to about 1:2:2 or about 1:1:1.

For example, the DES used in the methods and uses described herein may comprise or consist of betaine and urea, choline chloride and urea, glycerol and betane, glycerol and choline chloride, malic acid and choline, lactic acid and betaine or levulinic acid and betaine in a molar ratio of from about 4.1 to about 1:4, such as from about 2:1 to about 1:2 or about 1:1.

For example, the DES used in the methods and uses described herein may comprise or consist or consist essentially of pyruvic acid and betaine, sorbitol and betaine, urea and chlorine chloride, sorbitol and levulinic acid, proline and levulinic acid, betaine and levulinic acid, betaine and proline, betaine and glucose, proline and glucose, lysine and levulinic acid, glycerol and sorbitol, glycerol and lactic acid, glucose and levulinic acid, xylitol and levulinic acid, sorbitol and lactic acid, urea and betaine HCl, or glycerol and levulinic acid in a molar ratio of from about 4:1 to about 1:4, such as from about 2:1 to about 1:2 or about 1:1.

The DES may be obtained from a commercial source and added to water to form the extraction solution or the DES may be prepared separately by any known methods

Alternatively, the extraction solution comprising water and a DES may be prepared during the methods and uses described herein. As mentioned before, the DES may be formulated with or without water (such as solution comprising a DES wherein said solution is essentially free of water). In the last case, the solution comprising a DES wherein said solution is free of water (essentially free of water) may have other components, such as other solvents like ethanol, methanol, etc.

As such, the methods and uses described herein may include a step before step (i) of preparing the extraction solution comprising (a) combining two or more compounds selected from methylamines, organic acids, sugars, polyols, amino acids and urea to form a DES; and (b) diluting the product of (a) in water. In a particular embodiment of the methods and uses described herein, may include a step before step (i) of preparing the extraction solution comprising (a) combining two or more compounds selected from methylamines, organic acids, sugars, polyols, amino adds and urea to form a DES essentially free of water.

The water used for dilution of the DES formed in (b) may be from 0% to 90%, as from about 0.00001, 0.0001, 0.001, 0.01, 0.1, 0.5, 1, 5, 10, 15, 20, 30 or about 40% to about 90, 80, 70, 60 or about 50%. In a preferred embodiment, the water used in from about 5% to about 30%, such as about 10%, 20% 25% or about 30%.

For example, the methods and uses described herein may indude before step (i) the step of:

(a) combining betaine and urea or choline chloride and urea in a molar ratio of 1:2 to form a DES.

For example, the methods and uses described herein may include before step (i) the step of:
(a) combining betaine and urea or choline chloride and urea in a molar ratio of 1:2 to form a DES; and
(b) diluting the product of (a) in water.

Further combinations of DES may be: glycerol:betaine (2:1 molar ratio), glycerol:betaine (1:1 molar ratio), glycerol:betaine (1:2 molar ratio), glycerol:choline chloride (2:1 molar ratio), glycerol:choline chloride (1:1 molar ratio), glycerol:choline chloride (1:2 molar ratio), malic acid:choloride (1:1 molar ratio), lactic acid:betaine (3:1 molar ratio), lactic acid:betaine (2:1 molar ratio), lactic acid:betaine (1:1 molar ratio), levulinic acid:betaine (3:1 molar ratio), levulinic acid:betaine (2:1 molar ratio), levulinic acid:betaine (2:1 molar ratio), levulinic acid:betaine (2:1 molar ratio), levulinic acid:betaine (2:1 molar ratio), levulinic acid:betaine (2:1 molar ratio), pyruvic acid:betaine (2:1 mol), urea:betaine (2:1 molar rato), urea:betaine (1:1 molar ratio), urea:betaine HCl (2:1 mol), urea:choline chloride (2:1 molar ratio I) betaine:sorbitol (2:1 molar ratio), betaine:sorbitol (1:1 molar ratio), betaine:sorbitol (1:1 molar ratio), betaine:sorbitol (1:1 molar ratio), betaine:sorbitol (1.2 molar ratio) betaine:sorbitol (1:2 molar ratio), proline:levulinic acid (1:1 molar ratio), betaine:proline (1:1 molar ratio), betaine:proline (1:2 molar ratio), praline:glucose (3:1 molar ratio), proline:glucose (2:1 molar ratio), proline:glucose (1:1 molar ratio), proline:glucose (1:1 molar ratio), proline:glucose (1:2 molar ratio), betaine:glucose (3:1 molar ratio) betaine:glucose (2:1 molar ratio), betaine:glucose (1:1 molar ratio9, betaine:glucose (1:2 molar ratio), betaine:glucose (1:2 molar ratio), lysine:levulinic acid (1:1 molar ratio), lysine:levulinic acid (1:2 molar ratio), sorbitol:levulinic acid (1:1 molar ratio), sorbitol:levulinic acid (1:1 molar ratio), xylitol.levulinic acid (1:1 molar ratio), glucose:levulinic acid (1.1 molar ratio), glucose:lavulinic acid (1:2 molar ratio), glycerol:sorbitol (1:1 molar ratio), glycerol:lactic acid (1:1 molar ratio), sorbitol: lactic acid (1:1 molar ratio), or glycerol:levulinic acid (1:1 molar ratio).

Further combinations of DES and water may be: glycerol:betaine (2.1 molar ratio)+30% of water, glycerol:betaine (2:1 radar ratio)+25% of water, glycerol:betaine (2:1 molar ratio)+20% of water, glycerol:betaine (2:1 molar ratio)+10% of water, glycerol:betaine (1:1 molar ratio)+20% of water, glycerol:betaine (1:2 molar ratio)+30% of water, glycerol:choline chloride (2:1 molar ratio)+0% of water, glycerol:choline chloride (1:1 molar ratio)+10% of water, glycerol:choline chloride (1:2 molar ratio)+20% of water, malic acid:choline chloride (1:1 molar ratio)+30% of water, malic acid:choline chloride (1:1 molar ratio)+20% of water, lactic acid:betaine (3:1 molar ratio)+10% of water, lactic acid:betaine (2:1 molar ratio)+30% of water, lactic acid:betaine (2:1 molar ratio)+25% of water, lactic acid:betaine (2:1 molar ratio)+20% of water, acid:betaine (2:1 molar ratio)+10% of water, lactic acid:betaine (2:1 molar ratio)+5% of water, lactic acid:betaine (1:1 molar ratio)+10% of water, levulinic acid:betaine (3:1 molar ratio)+25% of water, levulinic acid:betaine (2:1 molar ratio)+30% of water, levulinic acid:betaine (2:1 molar ratio)+25% of water, levulinic acid:betaine (2:1 molar ratio)+20% of water, levulinic acid:betaine (2:1 molar ratio)+10% of water, levulinic acid:betaine (2:1 molar ratio)+5% water, pyruvio acid:betaine (2:1 mol)+25% of water, urea:betaine (2:1 mol) at 950 g/L in water, urea:betaine (1:1 mol) at 950 g/L in water, urea:betaine HCl (2.1 mol) at 0% water, urea:chloline chloride (2:1 mol) at 0% water, betaine:sorbitol (2:1 mol)+30% water, betaine:sorbitol (1:1 mol)+50 water, betaine:sorbitol (1.1 mol)+30% water, betaine:sorbitol (1:1 mol)+20% water, betaine:sorbitol (1:2 mol)+50% water, betaine:sorbitol (1:2 mol)+30% water, betaine:sorbitol (1:2 mol)+20% water, proline:levulinic acid (1:1 molar ratio)+20% water, betaine:proline (1:1 molar ratio)+30% water, betaine:proline (1:2 molar ratio)+30% water, proline:glucose (3:1 molar ratio)+20 % water, proline:glucose (2:1 molar ratio)+20% water, proline:glucose (1:1 molar ratio)+20 water, proline:glucose (1:1 molar ratio)+30% water, proline:glucose (1:2 molar ratio)+20% water, betaine:glucose (3:1 molar ratio)+20 % water, betaine:glucose (2:1 molar ratio)+20 % water, betaine:glucose (1:1 molar ratio)+20% water, betaine:glucose (1:2 molar ratio)+50% water, betaine:glucose (1:2 molar ratio)+30% water, betaine:glucose (1:2 molar ratio)+20% water, lysine:levulinic acid (1:1 molar ratio)+20% water, lysine:levulinic acid (1:2 molar ratio)+20% water, sorbitol:levulinic acid (1:1 molar ratio)+30% water, sorbitol:levulinic acid (1:1 molar ratio)+20% water, xylitol:levulinic acid (1:1 molar ratio)+20% water, glucose:levulinic acid (1:1 molar ratio)+30% water, glucose:levulinic acid (1:2 molar ratio)+30% water, glycerol:sorbitol (1:1 molar ratio)+20% water, glycerol:lactic acid (1:1 molar ratio)+20% water, sorbitol:lactic acid (1:1 molar ratio)+20% water, or glycerol:levulinio acid (1:1 molar ratio)+20% water.

It should be noted that the use of a DES as a hydrotrope is surprising and unexpected.

Thus, the present invention also provides the use of a DES as a hydrotrope.

It has been surprisingly and unexpectedly found that in the methods and uses of the present invention, the combination of two of more compounds selected from methylamines, organic acids, sugars, polyols, amino acids and urea in a molar ratio from about 4:1 to about 1:4, such as from about 2:1 to about 1:2 or about 1:1 provides a synergistic hydrotropic effect. For example, when the molar ratio of the two components is from about 1:0.5 to about 1.4, a synergistic hydrotropic effect is observed compared to the hydrotropic effect of the compounds on their own.

For example, the method of the invention may compose or consist of:

i) mixing plant biological material (such as from a plant of the Lamiaceae species, e.g. rosemary and/or sage, or such as from a plant of the Citrus genus e.g. C. sinensis, C. medica, C. reticulate, etc., or such as from a plant of the Zingiberaceae family, e.g., Curcuma longa) with an extraction solution comprising water and a DES; and

ii) removing any undissolved plant biological material from the solution obtained in (i);

iii) obtaining a flocculate and/or precipitate by adding water to and/or cooling the solution obtained in step (ii);

iv) collecting the resulting solid material obtained in step (iii) from the solution; and

v) optionally drying the solid material obtained in step (iv).

The use of the invention may comprise the use of an extraction solution comprising water and a DES to provide a biological extract and/or lipophilic, hydrophobic, oil soluble and/or non-water-soluble compounds from plant biological material material (such as from a plant of the Lamiaceae species, e.g. rosemary and/or sage, or such as from a plant of the Citrus genus e.g. C. sinensis, C. medica, C. reticulate etc., or such as from a plant of the Zingiberaceae family, e.g. Curcuma longa), wherein the use comprises:

i) mixing plant biological material (such as rosemary and/or sage and/or Curcuma longa, and/or Citrus sinensis) with an extraction solution comprising water and a DES; and

ii) removing any undissolved plant biological material from the solution obtained in (i);

iii) obtaining a flocculate and/or precipitate by adding water to and/or cooling the solution obtained in step (ii);

iv) collecting the resulting solid material obtained in step (iii) from the solution; and

v) optionally drying the solid material obtained in step (iv).

The concentration of the DES present in the extraction solution is at least the minimum hydrotropic concentration (MHC) of the DES. For example, from about 1 to about 20 times, such as from about 2 to about 16 times or from about 4 to about 8 times the minimum hydrotropic concentration (MHC).

The DES may be present in the extraction solvent at a concentration of about 99% or less, such as 90% or less or 80 or less by weight relative to the weight the extraction solvent, for example, the DES may be present in the extraction solvent at a concentration of about 70% of less or about 60% or less by weight relative to the weight of the extraction solvent or about 50% or less by weight relative to the weight of the extraction solvent, or about 40% or less by weight relative to the weight of the extraction solvent or about 30% or less by weight relative to the weight of the extraction solvent or about 20% or less by weight relative to the weight of the extraction solvent.

For example, the DES may be present in the extraction solvent at a concentration of from about 2% or about 2.5% to about 99% such as from about 5% to about 90% or from about 7.5% to about 80% by weight of the extraction solvent, or 10% to about 70% by weight or from about 15% to about 60% by weight relative to the weight of the extraction solvent.

The DES may be present in the extraction solvent at a concentration of from about 1 g/L to about 999 g/L.

In a particular embodiment of the method of the invention, in step 1) the extraction solution comprises a DES and less than about 0.5 of water, less than about 0.1, less than about 0.01, 0.001, 0.0001 or less than about 0.00001 of water.

In a particular embodiment of the methods or uses of the invention, in step i) the extraction solution comprising a DES is free of water.

In step (i) of the method of the invention, the biological material and the extraction solution comprising water and a DES may be mixed using such techniques known in the art, for example using stirring, maceration, percolation or infusion, such as magnetic or mechanical stirring, extrusion, high shear or rotor-stator-assisted extraction (for example at 1000-5000 rpm).

Stirring may be conducted at any suitable revolution per minute (rpm), for example, the stirring may be done from about 1 rpm or about 10 rpm or about 50 rpm to about 500 rpm. For mechanical stirring this may typically be done from about 1 rpm to 500 rpm, such as from about 10 rpm to about 200 rpm.

In step (i) of the method of the invention, the biological materiel and the extraction solution comprising water and a DES, or the extraction solution comprising a DES wherein said solution is free of water, or the extraction solution comprising a DES and less than about 0.5% of water, less than about 0.1, less than about 0.01, 0.001, 0.0001 or less than about 0.00001% of water, may be mixed at a temperature of from about 15° C. to about 100° C., such as from about 20° C. to about 60° C. or from about 20° C. to about 80° C. or about 25° C.

In step (i) of the method of the invention, the biological material and the extraction solution comprising water and a DES, or the extraction solution comprising a DES wherein said solution is free of water, or the extraction solution comprising a DES and less than about 0.5% of water, less than about 0.1, less than about 0.01, 0.001, 0.0001 or less than about 0.00001% of water, may be mixed at a pressure of from about 10 bar (1000 KPa) to about 1000 bar (100000 KPa) or from about 20 bar (2000 KPa) to about 100 bar (10000 KPa).

In step (i) of the method of the invention, the biological material and the extraction solution comprising water and a DES, or the extraction solution comprising a DES wherein said solution is free of water, or the extraction solution comprising a DES and less than about 0.5% of water, less than about 0.1, less than about 0.01, 0.001, 0.0001 or less than about 0.00001% of water, may be mixed for a duration of from about 1 minute to at least about 48 hours, to at least 24 hours, to at least about 10 hours, to at least about 5 hours, such as from about 5 minutes to about 1 hour or from about 5 minutes to about 30 minutes.

In step (ii), any solid biological material present in the solution obtained in step (i) may be removed by any means known in the art, for excample by filtration, static or dynamic decantation, and/or centrifugation.

In the method or use described above, the extraction solvent may recover at least 10%, at least 20%, at least 40% or at least 50%, or at least 60%, or at least 70%, or at least 80%, or at least 90%, such as from about 10% to about 80%, or from about 20% to about 60%, or from about 20% to about 80% of lipophilic, hydrophobic, oil soluble and/or non-water-soluble compounds of the biological material (such as plant biological material, algal biological material, animal biological material and/or prokaryotic biological material).

Compounds that have limited solubility in aqueous solutions, i.e. compounds that are lipophilic, hydrophobic, oil soluble and/or non-water-soluble that may be present in the biological extract of the invention include, but are not limited to, are phenolic compounds including phenolic acids (such as salycilic acid, rosmarinic acid or caffeic acid), phenolic esters, phenolic diterpenes (such as carnosic acid and its derivatives), flavonoids (such as hesperidin luteolin glucuronide), secoiridoids, curcuminoids (such as curcumin and its derivatives, sesquiterpenoids (such as turmerone, curcumadione, procurcumadiol or dehydrocurdione), diterpenoids (such as carnosic acid or hydroxycryptotanshinone), seponins, lignans, anthrequinone, glucosinolates, sulforaphane and isothiocyanates, triterpenoids (such as ursolic acid), sapogenins or carotenoids, and mixtures thereof, from the biological material (i.e. the plant and/or algal and/or and/or animal and/or prokaryotic biological material). For example, the biological extract may comprise one or more of the compounds listed above or mixtures thereof.

The water added in step (iii) reduces the concentration of the DES to near to or under the minimum hydrotropic concentration (MHC). The amount of water required to achieve this will depend on the MHC of the DES present in the aqueous solution (or the solution) and the amount of at least one hydrotropic agent present in the extraction solvent. For example, water may be added to dilute the concentration of the DES by a factor of from about 2 to about 1000, to about 100, to about 50, to about 40, to about 30, to about 20, such as by factor of from about 2 to about 20, such as from about 3 to about 10, such as from about 5 to 9, or from about 4 to about 6. For example, if the MHC of the DES was 20% by weight of the extraction solvent, sufficient water would be added to reduce the concentration of the DES to near to or below 20% by weight of the extraction solvent.

Reducing the concentration of the DES in the extraction solution to near to or under the MHC has the effect of causing any compounds present in the solution that are only soluble due to the presence of the DES to flocculate and/or precipitate from the solution. This solid material can then be collected.

For example, the method of the invention may comprise or consist of;

i) mixing plant biological material (such as from a plant of the Larniaceae sinensis, e.g. rosemary and/or sage, of such as from a plant of the Citrus genus e.g. C. sinensis C. medica, C. reticulate, or such as from a plant of the Zingiberaceae family, e.g. Carcuma longa) with an extraction solution comprising water (such as 50, 30, 25, 20, 10 or 0%) and a DES (such as urea and betaine, glycerol and betaine, pyruvic acid and betaine, choline chloride and urea, glycerol and choline chloride, malic acid and choline chloride, levulinic acid and betaine, lactic acid and betaine, sorbitel and levulinic acid, betaine and sorbitol, proline and levulinic acid, betaine and proline, betaine and glucose, proline and glucose, lysine and levulinic acid, glycerol and sorbitol, glycerol and lactic acid, glucose and levulinic acid, xylitol and levulinic acid, sorbitol and lactic acid, urea and betaine HCl, or glycerol and levulinic acid in a molar ratio of 4:1, 3:1 2:1, 1:1, 1:2, 1:3 or 1:4); and

ii) removing any undissolved plant biological material from the solution obtained in (i); iii) obtaining a flocculate and/or precipitate by adding water to and/or cooling the solution obtained in step (ii);

iv) collecting the resulting solid material obtained in step (iii) from the solution; and

v) optionally drying the solid material obtained in step (iv), wherein the water added in step (iii) to the solution obtained in step (ii) is from about 2 to about 100, such as about 2 to about 30, such as from about 4 to about 8, such as from about 8 to about 10 times the volume of the DES or the volume of the solution obtained in step (ii).

In a particular embodiment, in step i) the extraction solution comprising a DES is free of water. In another particular embodiment in step i) the extraction solution comprises a DES and less than about 0.5% of water, less than about 0.1, less than about 0.01, 0.001, 0.0001 or less than about 0.00001% of water.

The solid material that flocculate and/or precipitate are substantially compounds that are lipophilic, hydrophobic, oil soluble and/or non-water-soluble compounds present in the biological material and extracted using the method of the invention from said biological material that were soluble only due to the presence of the DES.

This flocculation/precipitation allows to collect the biological extract obtained using the method of the invention (i.e. lipophilic, hydrophobic, oil soluble and/or non-water-soluble compounds present in the biological material) since there is a creation of a solid form from the solution. This solid material can be collected very easily using technique known in the art. This permits a very easy, cost effective and efficient recuperation of the biological extract (i.e. lipophilic, hydrophobic, oil soluble and/or non-water-soluble compounds present in the biological material extracted using the method of the invention).

In step (iv), the solid material may be collected using such techniques known in the art, such as filtration, static or dynamic decantation, and/or centrifugation.

In step (v), the solid material may be dried using such techniques known in the art as previously defined.

The crude solid or semi-solid biological extract is enriched in, for example comprises about 2% or more, about 4% or more, about 5% or more or 10% or more or 20% or more by weight of the extract, compounds that are not usually soluble in aqueous solutions as defined above. For example, when the biological material plant material, such as rosemary, sage, Curcuma longa, or Citrus, the crude solid or semi-sold biological extract may be enriched in, for example comprise about 2% or more, about 4% or more, about 5% or more or 10% or more or 20% or more phenolic diterpenes, for example, carnosic acid, curcuminoids, curcumin, or hesperidin and/or its derivatives and/or other lipophilic, hydrophobic, oil soluble and/or non-water-soluble compounds present in the biological material as defined above.

For example, the method of the present invention may compose or consist of:

i) mixing plant biological material (such as from a plant of the Larniaceae species, e.g. rosemary and/or sage, or such as from a plant of the Citrus genus e.g. C. sinensis, C. medica, C. reticulate, or such as from a plant of the Zingiberaceae family, e.g. Curcuma longa) with an extraction solution comprising water (such as 50, 30, 25, 20, 10 or 0%, or such as less than about 0.5% of water, less than about 0.1, 0.01, 0.001, 0.0001 or less than about 0.00001% of water) and a DES;

ii) removing any undissolved biological material from the solution obtained in (i);

iii) obtaining a flocculate and/or precipitate by adding water to and/or cooling the solution obtained in step (ii);

iv) collecting the resulting solid material obtained in step (iii) from the solution; and

v) optionaily drying the solid material obtained in step (iv).

In step (iii), water may be added to the solution obtained in step (ii) at any suitable speed. For example, water may be added at a speed of from about 0.01 mL/s (for example, dropwise) to about 5 mL/s or more.

In step (iii), water may be added to the solution obtained in step (ii) at any temperature. For example, water may be added at a temperature of from about 15° C. to about 40° C., such as from about 20° C. to about 30° C. The water may be added to the solution obtained in step (ii) with cooling. For example, water may be added to the solution obtained in step (ii) while the solution is being cooled to a temperature of from about 0° C., to about 10° C., such as from about 2.5° C. to about 5° C. or water that has been pre-cooled to a temperature of from about 0° C. to about 10° C., such as from about 2.5° C. to about 5° C. may be used.

In step (iii), once the water has been added, the solution may be stirred to induce flocculation/precipitation. The solution may be stirred at any suitable speed. For example, the solution may be stirred at from about 130 rpm to about 500 rpm using a magnetic stirring or from about 1 rpm to about 500 rpm using a mechanical stirring.

In an aspect of the invention, step (iii) may be replaced with cooling the solution obtained in step (ii) causing solid material to precipitate from the solution (such as crystalize and separate from the solution).

Where step (iii) is replaced with cooling, the cooling of the solution obtained in step (ii) may be done at a temperature of from about 0° C. to about 10° C., such as from about 2.5° C. to about 5° C.

The method of the invention may additionally comprise step (vi), wherein in step (vi) the crude solid biological extract is washed from about 1 to about 3 times or more, such as to about 10, or about 100 times, with from about 1 to 100 volumes of water (such as from about 1, 10, 20, 30 or 40 to about 100, 90, 80, 70, 60 or 50 volumes of water, such as 2 to 4 volumes of water) and the resulting solid material is collected.

The inventors have surprisingly found that the yield of the crude (non washed) and/or purified (washed) solid biological extract can be further increased if a salt is added in the water of step (iii) and/or during the washing step (vi). Thus, in a further embodiment, the water used for washing in step (vi) and/or the water of step (iii) may comprise one or more said such as sodium chloride. The amount of salt (such as sodium chloride) added to the water of step (iii) and/or step (vi) can be from about 0.1 g/L to about 10 g/L, such as from about 2 to about 5 g/L, such as about 3 g/L.

A method of the invention comprising step (vi) provides a purified solid biological extract. lf required, the purified biological solid may be dried to reduce/remove any residual water present in the extract.

Any suitable drying techniques known in the art may be used, such as, but not limited to, freeze-, spray-, oven-, heat- or vacuum-drying.

For example, the present invention provides a method of providing a purified solid biological extract comprising:
i) combining biological material (such as plant biological material, algal biological material, animal biological material and/or prokaryotic biological material) with an extraction solution comprising water (such as 50, 30, 25, 20, 10 or 0% of water, or such as less than about 0.5% of water, less than about 0.1, 0.01, 0.001, 0.0001 or less than about 0.00001%) and a DES (such as urea:betaine, glycerol and betaine, pyruvic acid and betaine, choline chloride and urea, glycerol and choline chloride, malic acid and choline chloride, levulinic acid and betaine, lactic acid and betaine, sorbitol and levulinic acid, betaine and sorbitol, proline and levulinic acid, betaine and proline, betaine and glucose, proline and glucose, lysine and levulinic acid, glycerol and sorbitol, glycerol and lactic acid, glucose and levulinic acid, xylitol and levulinic acid, sorbitol and lactic acid, urea and betaine HCl, or glycerol and levulinic acid); and;

ii) removing any undissolved biological material from the solution obtained in (i);

iii) obtaining a flocculate and/or precipitate by adding water to and/or cooling the solution obtained in step (ii);

iv) collecting the solid material obtained in step (iii) from the solution;

v) optionally drying the solid material obtained in step (iv);

vi) washing the solid obtained in step (iv) or (v) from about 1 to about 10 times with 1 to 1000 volumes of water and collecting the resulting solid material; and

vii) optionally drying the solid material obtained in step (vi).

The present invention also provides the use of an extraction solution comprising water and a DES to provide a biological extract and/or lipophilic, hydrophobic, oil soluble and/or non-water-soluble compounds from biological material (such as plant biological material, algal biological material, animal biological material and/or prokaryotic biological material), wherein the use comprises:

i) mixing biological material (such as plant biological material, algal biological material, animal biological material and/or prokaryotic biological material) with an extraction solution comprising water (such as 50, 30, 25, 20, 10 or 0% of water, or such as less than about 0.5 of water, less than about 0.1, 0.01, 0.001, 0.0001 or less than about 0.00001% water) and a DES; and

ii) removing any undissolved biological material from the solution obtained in (i);

iii) obtaining a flocculate and/or precipitate by adding water to and/or cooling the solution obtained in step (ii);

iv) collecting the resulting solid material obtained in step (iii) from the solution; and

v) optionally drying the solid material obtained in step (iv);

vi) washing the solid obtained in step (iv) or (v) from about 1 to about 10 times with 1 to 1000 volumes of water and collecting the resulting solid material; and

vii) optionally drying the solid material obtained in step (vi).

The biological material and DES are as defined previously.

The washing in step (vi) reduces the concentration of residual DES present in the extract. Surprisingly and unexpectedly, the present inventors have found that the washing step (vi) does not reduce the concentration of lipophilic/non-water-soluble compounds present in the purified solid biological extract and in fact may increase the concentration of such compounds present in the extract. For example, where the biological material is plant material, such as rosemary and/or sage, or Curcuma longa, and biological extract is enriched in carnosic acid and/or its derivatives or curcuminoids and/or curcumin, and/or other lipophilic, hydrophobic, oil soluble and/or non-water-soluble compounds present in the biological material, washing the extract does not significantly reduce the concentration of carnosic acid and/or its derivatives or curcuminoids and/or curcumin, and/or other lipophific, hydrophobic, oil soluble and/or non-water-soluble compounds present in the biological material present within the purified extra and in fact may increase the concentration of such compounds present in the extract.

Thus, in the methods and uses described herein that provide a purified solid biological extract, the biological extract obtained may comprise less than about 5% by weight of the extract of the DES, such as less than 2% or less than 1% or less than 0.1%, or less than 0.04% of the DES, in particular at least one DES described previously. For example, the biological extract obtained may be substantially free of DES.

As used herein, the term “substantially free” means that the extract being described may contain small (for example up to 0.1% or 0.01% or 0.001% or 0.0001% by weight of the extract) of the DES, provided that the presence of DES, does not affect the essential properties of the extract.

The method of the present invention may comprise or consist of;

i) mixing plant biological material (such as from a plant of the Lamiaceae species, e.g. rosemary and/or sage, or such as from a plant of the Citrus genus e.g. C. sinensis, C. medica, C. reticulate, or such as from a plant of the Zingiberaceae family, e.g. Curcuma longa) with an extraction solution comprising water (such as 50, 30, 25, 20, 10 or 0% of water, or such as less than about 0.5% water, less than about 0.1, 0.01, 0.001, 0.0001 or less than about 0.00001%) and a DES;

ii) removing any undissolved biological material from the solution obtained (i);

iii) obtaining a flocculate and/or precipitate by adding water to and/or cooling the solution obtained in step (ii);

iv) collecting the resulting solid material obtained in step (iii) from the solution; and

v) optionally drying the solid materiel obtained in step (iv)

vi) optionally washing the solid obtained in step (iv) or (v) from about 1 to about 10 times with water and collecting the resulting solid material; and

vii) optionally drying the solid material obtained in step (vi).

If required, before step (i) the biological material (i.e. plant and/or algal and/or animal and/or prokaryotic biological material) may be dried and/or ground e.g. into a powder, before being mixed with an extraction solution comprising water and a DES.

Where the extract is obtained using a method or use comprising steps (i) to (vii) the extract may be considered to be a purified biological extract.

The purified biological extract obtained via the method or use described herein comprising steps (i) to (vii) may comprise about 10% or more, about 20% or more, about 40 or more, or about 60% or more by weight of the dried purified extract of compounds that have limited solubility in aqueous solutions, i.e. compounds that are lipophilic, hydrophobic, oil soluble and/or non-water-soluble. For example, the purified biological extract may comprise from about 10% to about 80% by weight of the extract of compounds that have limited solubility in aqueous solutions, i.e. compounds that are lipophilic, hydrophobic, oil soluble and/or non-water-soluble, or from about 20% to about 60% by weight of the extract.

For example, the extract may comprise at least about to 10% by weight of the dried purified extract of phenolic compounds including phenolic acids (such as carnosic acid, rosmarinic acid or caffeic acid), phenolic esters, phenolic diterpenes (such as salycilic acid and its derivatives), flavonoids (such as hesperidin luteolin glucuronide), secoiridoids, curcuminoids (such as curcumin and its derivatives, demethoxycurcumin or bisdemethoxycurcumin), bixin, capsaicinoids, cannabinoids, pyranoanthocyanins, stilbenes, phenolic alcohols, phenolic lipids (such as shogaol or ubiquinol), sylimarins, alkaloids, lipids, phenylpropanoids, coumarin (such as dimethoxycoumarin), organic acids (such as malik acid or tartaric acid), terpenoids including monoterpenoids, sesquiterpenoids (such as turmerone, curcumadione, procurcumadiol or dehydrocurdione), diterpenoids (such as carnosic acid or hydroxycryptotanshinone), saponins, lignans, anthraquinone, glucosinolates, sulforaphane and isothiocyanates, triterpenoids (such as ursolic acid), sapogenins or carotenoids, and mixtures thereof, from the biological material (i.e. the plant and/or algal and/or and/or animal and/or prokaryotic biological material). For example, the biological extract may comprise one or more of the compounds listed above or mixtures thereof.

For the avoidance of doubt, preferences, options, particular features and the like indicated for a given aspect, feature or parameter of the invention should, unless the context indicates otherwise, be regarded as having been disclosed in combination with any and all other preferences, option, particular features and the like as indicated for the same or other aspects, features and parameters of the invention.

The term “about ” as used herein, e.g. when referring to a measurable value (such as an amount of weight of a particular component in the composition or reaction mixture), refers to variations of ±20%, ±10%, ±5%, ±1%, ±0.5%, or particularly, ±0.1%, of the specified amount.

In some aspects of the invention, the method of the invention may consist or consist essentially of the step described herein.

For the avoidance of doubt, in this specification when we use the term “comprising” or “comprises” we mean that the method being described must contain the listed step(s) but may optionally contain additional steps. When we use the term “consisting essentially of” or “consists essentially of” we mean that the method of the invention being described must contain the listed step(s) and may also contain minor additional steps provided that the additional steps do not affect the essential properties of the method. When we use the term “consisting of” or “consists of” we mean that the method of the invention being described must cpntain the listed step(s) only.

Biological Extract

The present invention also provides a biological extract obtained using the method or uses described previously, which may be referred to hereafter as the “extract of the invention”.

The biological extract may be, for example, a crude solid or semi-solid biological extract or a purified biological extract.

The biological extract may be enriched in compounds that have limited solubility in aqueous solutions, i.e. compounds that are lipophilic, hydrophobic, oil soluble and/or non-water-soluble.

For example, the crude biological extract may comprise about 2% or more, about 5% or more, about 10% or more, about 20% or more, or about 40% or more by weight of the extract of compounds that have limited solubility in aqueous solutions, i.e. compounds that are lipophilic, hydrophobic, oil soluble and/or non-water-souble, i.e. the crude biological extract may comprise from about 2% to about 60% by weight of the extract of compounds that have limited solubility in aqueous solutions, i.e. compounds that are lipophilic, hydrophobic, oil soluble and/or non-water-soluble or from about 5% to about 40% by weight of the extract of compounds that have limited solubility in aqueous solutions, i.e. compounds that are lipophilic, hydrophobic, oil soluble and/or non-water-soluble.

The purified biological extract may comprise about 10% or more, about 20% or more, about 40% or more, or about 60%, or about 70% or more by weight of the dried purified extract of compounds that have limited solubility in aqueous solutions, i.e. compounds that are lipophilic, hydrophobic, oil soluble and/or non-water-soluble, i.e. the purified biological extract may comprise from about 10% to about 80% by weight of the extract of compounds that have limited solubility in aqueous soutions, i.e. compounds that are lipophilic, hydrophobic, oil soluble and/or non-water-soluble, or from about 20% to about 60% by weight of the extract.

For example, the crude biological extract may comprise about 2% more, about 5% or more, about 10% or more, about 20% or more, or about 40% or more by weight of the extract of phenolic compounds including phenolic acids (such as salycilic acid, rosmarinic acid or caffeic acid), phenolic esters, phenolic diterpenes (such as camosic acid and its derivatives), flavonoids (such as hesperidin or luteolin glucuronide), secoiridoids, curcuminoids (such as curcumin and its derivatives, demethoxycurcumin or bisdemethoxycurcumin), bixin, capsaicinoids, cannabinoids, pyranoanthocyanins, stilbenes, phenolic alcohols, phenolic lipids (such as shogaol or ubiquinol), sylimarins, alkaloids, lipids, phenyipropanoids, coumarin (such as dimethoxycoumarin), organic acids (such as malic acid or tartaric acid), terpenoids including monoterpenoids, sesquiterpenoids (such as turmerone, curcumadione, procurcumadiol or dehydrocurdione), diterpenoids (such as carnosic acid or hydroxycryptotanshinone), saponins, lignans, anthraquinone, glucosinolates, sulforaphane and isothiocyanates, triterpenoids (such as ursolic acid), sapogenins or carotenoids, and mixtures thereof, i.e. the crude biological extract may comprise from about 2% to about 60%, or from about 5% to about 40% by weight of the crude extract of phenolic compounds including phenolic acids (such as salycilic acid, rosmarinic acid or caffeic acid), phenolic esters, phenolic diterpenes (such as carnosic acid and its derivatives), flavonoids (such as hesperidin or luteolin glucuronide), secoiridoids, curcuminoids (such as curcumin and its derivatives, demethoxycurcumin or bisdemethoxycurcumin), bixin, capsaicinolds, cannabinoids, pyranoanthocyanins, stilbenes, phenolic alcohols, phenolic lipids (such as shogaol or ubiquinol), sylimarins, alkaloids, lipids, phenylpropanoids, coumarin (such as dimethoxycoumarin), organic acids (such as malic acid or tartaric acid), terpenoids including monterpenoids, sesquiterpenoids (such as turmerone, curcumadione, procurcumadiol or dehydrocurdione), diterpenoids (such as carnosic acid or hydroxycryptotanshinone), saponins, lignans, anthraquinone, glucosinolates, sulforaphane and isothiocyanates, triterpenoids (such as ursolic acid), sapogenins or carotenoids, and mixtures thereof.

The purified biological extract may comprise about 10% or more, about 20% or more, about 40% or more, or about 60% or more, or about 70% or more by weight of the dried purified extract of carnosic acid and/or hesperidin, i.e. the purified biological extract may comprise from about 10% to about 80% by weight of the extract of carnosic acid and/or hesperidin, or from about 20% to about 60% by weight of the extract.

The purified biological extract may comprise about 10% or more, about 20% or more, about 40% or more, or about 60% or more, or about 70% or more by weight of the dried purified extract of curcuminoids and/or curcumin, i.e. the purified biological extract may comprise from about 10% to about 80% by weight of the extract of or curcumin and/or curcuminoids from about 20% to about 60% by weight of the extract.

In the biological extract of the invention, the percentage of DES remaining may be about less than about 5% by weight of the extract of the DES, such as less than 2% or less than 1% or less than 0.1% or less than about 0.04% or less of the DES by weight of the extract, in particular at least one DES described previously. For example, the biological extract obtained may be substantially free of DES.

As used herein, the term “substantially free” means that the extract being described may contain small (for example up to 0.1% or 0.01% or 0.001% or 0.0001% by weight of the extract) of the DES, provided that the presence of DES, does not affect the essential properties of the extract.

Uses of the Biological Extract of the Invention

The biological extract of the invention may be used to provide phenolic compounds including phenolic acids (such as salycilic acid, rosmarinic aci or caffeic acid), phenolic esters, phenolic diterpenes (such as carnosic acid and its derivatives), flavonoids (such as hesperidin or luteolin glucuronide), secoiridoids, curcuminoids (such as curcumin and its derivatives), bixin, capsaicinoids, cannabinoids, pyranoanthocyanins, stilbenes, phenolic alcohols, phenolic lipids (such as shogaol or ubiquinol), sylimarins, alkaloids, lipids, phenylpropanoids, coumarin (such as dimethoxycoumarin), organic acids (such as malic acid or tartaric acid), terpenoids including monoterpenoids, sesquiterpenoids (such as turmerone, curcumadione, procurcumadiol or dehydrocurdione), diterpenoids (such as carnosic acid or hydroxycryptotanshinone), saponins, lignans, anthraquinone, glucosinolates, sulforaphane and isothiocyanates, triterpenoids (such as ursolic acid), sapogenins, saponins, or carotenoids, and mixtures thereof from the biological material (i.e. the plant and/or algal and/or animal and/or prokaryotic biological material). For example, the biological extract may comprise one or more of the compounds listed above or mixtures thereof that may be used as natural biological flavourings and taste modifiers, sweetener, fragrances, biocides, antimicrobials, proteins, enzymes, colourings, pigments, surfactants, antioxidants, chelatant, emulsifier, texturizers, vitamins, and/or bioactives of nutritional, cosmetic or pharmaceutical interest.

For example, the biological extract may be high in compounds that provide anti-oxidant and/or anti-microbial activity (e.g. anti-bacterial activity) and/or anti-inflammatory activity. Thus, the present invention provides a biological extract comprising antioxidants obtained from pant algal and/or and/or animal and/or prokaryotic biological material obtained by the methods described herein.

The present invention also provides the use of a biological extract obtained by the methods described herein as an anti-oxidant. The anti-oxidant extract may be used in the compositions and/or products as described below.

Thus, the present invention provides a biological extract comprising one or more anti-inflammatory, colour or pigments, vitamin, surfactant, flavouring agent, fragrance and/or taste modifiers obtained from plant and/or algal and/or and/or animal and/or prokaryotic biological material obtained by the methods described herein.

The present invention also provides the use of a biological extract obtained by the methods described herein as an anti-inflammatory, colour or pigments, vitamin, surfactant, flavouring agent, fragrance and/or taste modifiers. The anti-inflammatory, colour or pigments, vitamin, surfactant, flavouring agent, fragrance and/or taste modifiers extract may be used in the compositions and/or products as described below.

The present invention also provides the use of a biological extract (such as turmeric extract comprising curcumin, demethoxycurcumin or bisdemethoxycurcumin) obtained by the methods described herein as an anti-inflammatory for joint health, cognition (neuroinflammation), cardiometabolic, sport recovery, and/or digestive health.

Thus, the present invention also provides a biological extract comprising anti-microbial (e.g. anti-bacterial), anti-inflammatory, vitamin, colour or pigments, surfactant, flavouring agent, fragrance and/or taste modifiers compounds obtained from plant and/or algal and/or animal and/or prokaryotic biological material obtained by the methods described herein.

The present invention also provides the use of a biological extract obtained by the methods described herein as an anti-microbial (e.g. anti-bacterial), anti-inflammatory, colour or pigments, vitamin, surfactant, flavouring agent, fragrance and/or taste modifiers. The anti-microbial extract (e.g, anti-bacterial) or the anti-inflammatory, colour or pigments, vitamin, surfactant, flavouring agent, fragrance and/or taste modifiers may be used in the compositions and/or products as described below.

The biological extract of the invention (which may be anti-oxidant and/or anti-microbial (e.g. anti-bacterial), an anti-inflammatory, a vitamin, a colour or pigments, a surfactant, a flavouring agent, a fragrance and/or a taste modifiers) may be used to provide a nutraceutical composition, a dietary or food product for humans or animals (such as functional food compositions, i.e. food, drink, feed or pet food or a food, drink, feed or pet food supplements), a nutritional supplement, a fragrance or flavouring, a pharmaceutical (pharmaceutical compositions or formulations), a veterinary composition, an oenological or a cosmetic formulation.

The nutraceutical composition, dietary or food product for humans or animals (such as functional food compositions, i.e. food, drink, feed or pet food or a food, drink, feed or pet food supplements), nutritional supplement, fragrance or flavouring, pharmaceutical (pharmaceutical compositions or formulations), veterinary composition, oenological or cosmetic formulation may be administered orally or parenterally, or be for topical, rectal, nasal, auricular, vaginal and/or ocular application.

The present invention therefore provides a biological extract for use in nutraceutical compositions, dietary or food products for humans or animals (such as functional food compositions, i.e. food, drink, feed or pet food or a food, drink, feed or pet food supplements), nutritional supplements, fragrances or flavourings, pharmaceuticals (pharmaceutical compositions or formulations), veterinary compositions, oenological or cosmetic formulations.

The present invention also provides nutraceutical compositions, dietary or food products for humans or animals (such as functional food compositions, i.e. food, drink, feed or pet food or a food, drink, feed or pet food supplements), nutritional supplements, fragrances or flavourings, pharmaceuticals (pharmaceutical compositions or formulations), veterinary compositions, oenological or cosmetic formulations comprising the biological extract, and optionally one or more pharmaceutically/veterinary acceptable ingredients, such as excipients or carriers or (functional) food acceptable ingredients and mixtures thereof, as appropriate.

If necessary, the biological extract may be combined with other biologically active compounds within a nutraceutical composition, a dietary or food product for humans or animals (such as a functional food composition, i.e. a food, a drink, a feed or pet food or a food, drink, feed or pet food supplement), a nutritional supplement, a fragrance or flavouring, a pharmaceutical (pharmaceutical composition or formulation), a veterinary composition, an oenological or cosmetic formulation.

As used herein, references to pharmaceutically or veterinary acceptable excipients may refer to pharmaceutically or veterinary acceptable adjuvants, diluents and/or carriers as known to those skilled in the art.

Food acceptable ingredients include those known in the art (including those also referred to herein as pharmaceutically acceptable excipients) and can be natural or non-natural, i.e. their structure may occur in nature or not. In certain instances, they can originate from natural compounds and be modified before use (e.g. maltodextrin).

In an embodiment, the biological extract obtained by the methods described herein is rich in carnosic acid and is formulated with a food grade oil such as sunflower, olive oil, corn oil or rapeseed oil. In a preferred embodiment, the extract rich in carnosic acid is formulated with sunflower oil at a concentration of about 1 to 30%, such as from about 20 to 30%, such as from about 1 to 10%, such as from about 4 to 5%.

In another embodiment, the biological extract obtained by the methods described herein (such as an extract rich in carnosic acid or an extract rich in carnosic acid formulated in sunflower oil) is used for the manufacture of a food or beverage (such as a mayonnaise, a meat, etc.). In a preferred embodiment, the biological extract (such as an extract rich in carnosic acid or an extract rich in carnosic acid formulated in sunflower oil) is diluted from 10 to 100 times or 1000 or 10000 times.

By “pharmaceutically or veterinary acceptable” we mean that the additional components of the composition are generally safe, non-toxic, and neither biologically nor otherwise undesirable. For example, the additional components are generally sterile and pyrogen free. Such components must be “acceptable” in the sense of being compatible with the extract of the invention and not deleterious to the recipients thereof. Thus, “pharmaceutically acceptable excipients” includes any compound(s) used in forming a part of the formulation that is intended to act merely as an excipient, i.e. not intended to have biological activity itself.

The skilled person will understand that extracts of the invention (e.g. in the form of compositions, such as pharmaceutical or veterinary compositions) may be administered to a patient or subject (e.g. a human or animal patient or subject) by any suitable route, such as by the oral, rectal, nasal, pulmonary, buccal, sublingual, transdermal, intracisternal, intraperitoneal, or parenteral (including subcutaneous, intramuscular, intrathecal, intravenous and intradermal) route.

Extracts of the invention may be administered orally. In such instances, pharmaceutical or veterinary compositions according to the present invention may be specifically formulated for administration by the oral route.

Pharmaceutical or veterinary compositions for oral administration include solid dosage forms such as hard or soft capsules, tablets, troches, dragees, pills, lozenges, powders and granules. Where appropriate, they can be prepared with coatings such as enteric coatings, or they can be formulated so as to provide controlled release of the active ingredient, such as sustained or prolonged release, according to methods well known in the art.

Liquid dosage forms for oral administration include solutions, emulsions, aqueous or oily suspensions, syrups and elixirs.

Compositions (e.g. pharmaceutical or veterinary or food compositions) described herein, such as those intended for oral administration, may be prepared according to methods known to those skilled in the art, such as by mixing the components of the composition together.

The compositions of the invention may contain one or more additional ingredients, such as food ingredients or pharmaceutical ingredients and excipients, such as sweetening agents, flavouring agents, colouring agents and preserving agents. The compositions of the invention may contain the active ingredient(s) in admixture with non-toxic pharmaceutically acceptable excipients (or ingredients) which are suitable for the manufacture of tablets. These excipients (or ingredients) may, for example, be: inert diluents, such as calcium carbonate, sodium carbonate, lactose, calcium phosphate or sodium phosphate; granulating and disintegrating agents, for example, corn starch, maltodextrin or alginic acid; binding agents, for example, starch, gelatine or acacia; or lubricating agents, for example magnesium stearate, stearic acid, talc and mixtures thereof.

Solid compositions of the invention may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Liquid compositions of the invention may be contained within a capsule, which may be uncoated or coated as defined above.

Suitable pharmaceutical or veterinary carriers include inert solid diluents or fillers, sterile aqueous solutions and various organic solvents. Examples of solid carriers are lactose, terra alba, sucrose, cyclodextrin, maltodextrin, talc, gelatin, agar, pectin, acacia, magnesium stearate, stearic acid, arabic gum, modified starch and lower alkyl ethers of cellulose, saccharose, silica and mixtures thereof. Examples of liquid carriers are syrup, peanut oil, olive oil, phospholipids, fatty acids, fatty acid amines, polyoxyethylene and water.

Moreover, the carrier or diluent may include any sustained release material known in the art, such as glyceryl monostearate or glyceryl distearate, alone or mixed with a wax.

Suitable pharmaceutical carriers include inert solid diluents or fillers, sterile aqueous solutions and various organic solvents. Examples of solid carriers are lactose, terra alba, sucrose, cyclodextrin, maltodextrin, dextrin, talc, gelatine, agar, pectin, acacia, magnesium stearate, magnesium hydroxide, stearic acid, arabic gum, modified starch and lower alkyl ethers of cellulose, saccharose, silicon dioxide. Examples of liquid carriers are syrup, vegetables oils, phospholipids, fatty acids, fatty acid amines, polyoxyethylene and water. Moreover, the carrier or diluent may include any sustained release material known in the art, such as glyceryl monostearate or glyceryl distearate, alone or mixed with a wax.

The term “carrier” as used herein, may also refer to a natural product or a product originating from nature that has been transformed or modified so that it is distinct from the natural product from which it originated, such as maltodextrin.

Depending on the disorder, and the subject, to be treated, as well as the route of administration, extracts of the invention may be administered at varying doses (i.e. therapeutically effective doses, as administered to a patient in need thereof). In this regard, the skilled person will appreciate that the dose administered to a mammal, particularly a human, in the context of the present invention should be sufficient to affect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the age, condition, body weight, sex and response of the patient to be treated, and the stage/severity of the disease.

The pharmaceutical or veterinary or food compositions comprise an extract of the invention in a therapeutically effective amount. As used herein, the term “effective amount” is synonymous with “therapeutically effective amount”, “effective dose”, or “therapeutically effective dose” and when used in the present invention refers to the minimum dose of the extract of the invention necessary to achieve the desired therapeutic effect and includes a dose sufficient to reduce a symptom associated with inflammation. Effectiveness in treating the diseases or conditions described herein can be determined by observing an improvement in an individual based upon one or more clinical symptoms, and/or physiological indicators associated with the condition. An improvement in the diseases or conditions described herein also can be indicated by a reduced need for a concurrent therapy.

Additionally, where repeated administration of the extract of the invention is used, an effective amount of the extract of the invention will further depend upon factors, including, without limitation, the frequency of administration, the half-life of the extract of the invention, or any combination thereof.

The amount of the biological extract nutraceutical compositions, dietary or food products for humans or animal (such as functional food compositions, i.e, food, drink, feed or pet food or a food, drink, feed or pet food supplements), nutritional supplements, fragrances or flavourings, pharmaceuticals (pharmaceutical compositions or formulations), veterinary compositions, oenological or cosmetic formulations will vary depending on the application.

Typically, the amount of biological extract present in nutraceutical compositions, dietary or food products for humans or animals (such as functional food compositions, i.e. food, drink, feed or pet food or a food, drink, feed or pet food supplements), nutritional supplements, fragrances or flavourings, pharmaceuticals (pharmaceutical compositions or formulations), veterinary compositions, oenological or cosmetic formulations will be from about 0.0001% to about 100% by weight of the nutraceutical composition, dietary or food product for humans or animals (such as functional food compositions, i.e. food, drink, feed or pet food or a food, drink, feed or pet food supplements), nutritional supplement, fragrance or flavouring, pharmaceutical (pharmaceutical composition or formulation), veterinary composition, oenological or cosmetic formulation, such as from about 0.001% to about 50% or from about 0.01% to about 30%.

Pharmaceutical or veterinary or food compositions of the invention may consist of or consist essentially of the extract of the invention, and optionally a carrier.

For the avoidance of doubt, in this specification when we use the term “comprising” or “comprises” we mean that the extract or composition being described must contain the listed ingredient(s) but may optionally contain additional ingredients. When we use the term “consisting essentially of” or “consists essentially of” we mean that the extract or composition being described must contain the listed ingredient(s) and may also contain small (for example up to 5% by weight, or up to 1% or 0.1% by weight) of other ingredients provided that any additional ingredients do not affect the essential properties of the extract or composition. When we use the term “consisting of” or “consists or” we mean that the extract or composition being described must contain the listed ingredient(s) only.

### BRIEF DESCRIPTION OF THE FIGURES

FIG. 1. Effect of different betaine:urea mixtures on the solubility of the dye “disperse red 13” in aqueous solution at room temperature as measured by the absorbance at 525 nm. All solutions have been prepared in distilled water without any control of the pH. Results are expressed as the mean±Sd of a triplicate experiment (independent). Insert: Sₘₐₓ as a function of the urea-to-betaine molar fraction (%).

FIG. 2. Effect of different betaine:urea mixtures on the solubility of disperse red 13 in aqueous solution at room temperature as measured by the absorbance at 525 nm. All solutions have been prepared in pH 7 phosphate buffer solution. When necessary, the final hydrotropic solution have been adjusted to pH 7.0 with concentrated HCl. Added HCl volumes were less than 1% of the total volume in all tested mixtures, so the concentrations were not corrected. Results are expressed as the mean±Sd of a triplicate experiment (independent) insert; Sₘₐₓ as a function of the urea-to-betaine molar fraction (%).

FIG. 3. Effect of different choline chloride:urea mixtures on the solubility of disperse red 13 in aqueous solution at room temperature as measured by the absorbance at 525 nm. All solutions have been prepared in distilled water without any control of the pH. Results are expressed as the mean±Sd of a triplicate experiment (independent), insert: Sₘₐₓ as a function of the urea-to-choline chloride molar fraction (%).

FIG. 4. Sₘₐₓ of carnosic acid as a function of the urea-to-betaine molar fraction (%).

FIG. 5. Sₘₐₓ of hesperidin as a funtion of the urea-to-betaine molar fraction (%).

FIG. 6. Sₘₐₓ of curcumine as a function of the urea-to-betaine molar fraction (%).

FIG. 7 Recovery rate of carnosic acid (CA) extraction from ground rosemary leaves (A), CA content in the final crude (non-washed) extracts (B), and mass yield (C) obtained by using a 1:1 or a 2:1 urea:betaine deep eutectics at room temperature (RT) for 0.5, 1, 2, 3, 7, or 16 hours. All solvents comprise 950 g/L of DES in water. For all extractions, the plant:solvent weight ratio was 1:10 (denoted 10 M) and the precipitation was obtained using nine volumes of water at room temperature.

FIG. 8. Recovery rate of carnosic acid (CA) extraction from ground rosemary leaves (A), CA content in the final purified (washed) extracts (B), and mass yield (C) obtained by using a 1:1 or a 2:1 urea:betaine deep eutectics at room temperature (RT) for 0.5, 1, 2, or 3 hours. All solvents comprise 950 g/L of DES in water. The plant:solvent weight ratios were 1:10 (denoted 10 M), 1:15 (denoted 15 M), or 1:20 (denoted 20 M), and the precipitation was obtained using nine volumes of water at room temperature. The extracts were washed 1, 2 or 5 times.

FIG. 9. Recovery rate of carnosic acid (CA) extraction from ground rosemary leaves (A), CA content in the final crude (non-washed) extracts (B), and mass yield (C) obtained by using a 1:2 betaine:levulinic acid deep eutectics containing 10% of water at room temperature (RT) for 1 hour. The plant:solvent weight ratios were 1:10 (denoted 10 M) or 1:15 (denoted 15 M) and the precipitation was obtained for both conditions using nine volumes of water at room temperature.

FIG. 10. Recovery rate of carnosic acid (CA) extraction from ground rosemary leaves (A), CA content in the final purified (washed) extracts (B), and mass yield (C) obtained by using 1:2 betaine:levulinic acid deep eutectics containing 5, 10, 20, 25, or 30% of water at room temperature (RT) for 0.25, 0.5, 1, 2, or 3 hours. The plant:solvent weight ratios were 1:6 (denoted 6 M), 1:8 (denoted 8 M), 1:10 (denoted 10 M), or 1:15 (denoted 15 M). The precipitation was obtained using 1.5, 2, 3, 4, or 9 volumes of water at room temperature or at 4° C.

FIG. 11. Recovery rate of carnosic acid (CA) extraction from ground rosemary leaves (A). CA content in the final crude (non-washed) extracts (B), and mass yield (C) obtained by using a 1:1 or a 1:2 betaine:lactic acid deep eutectics containing 10% of water at room temperature (RT) for one hour. The plant:solvent weight ratios were 1:10 (denoted 10 M), or 1:15 (denoted 15 M) and the precipitation was obtained using nine volumes of water at room temperature.

FIG. 12. Recovery rate of carnosic acid (CA) extraction from ground rosemary leaves (A), CA content in the final purified (washed) extracts (B), and mass yield (C) obtained by using 1:2 or 1:3 betaine:lactic acid deep eutectics containing 5, 10, 20, or 25% of water at room temperature (RT) for one hour. The plant:solvent weight ratios were 1.8 (denoted 8 M), 1:10 (denoted 10 M), or 1:15 (denoted 15 M) and the precipitation was obtained using 4 or 9 volumes of water at room temperature. The first sample of the figure (from left) has been obtained after filtration of the precipitation waters on a 2 μm filter. The others have been centrifuged.

FIG. 13. Recovery rate of carnosic acid (CA) extraction from ground rosemary leaves (A), CA content in the final crude (non-washed) or purified (washed) extracts (B), and mass yield (C) obtained by using 1:2 betaine:glycerol deep eutectics containing 20 or 25 % of water at room temperature (RT) for one hour. The plant:solvent weight ratio was 1:10 (denoted 10 M) and the precipitation was obtained using 4 or 9 volumes of water at room temperature.

FIG. 14. Recovery rate of carnosic acid (CA) extraction from ground rosemary leaves (A). CA content in the final purified (washed) extracts (B), and mass yield (C) obtained by using 1:1, 1:2, or 2:1 betaine:sorbitol deep eutectics containing 20, 30, or 50% of water at room temperature (RT) for 0.5, 1 or 2 hours. The plant:solvent weight ratios were 1:8 (denoted 8 M), or 1:10 (denoted 10 M) and the precipitation was obtained using four volumes of water at room temperature.

FIG. 15. Recovery rate of carnosic acid (CA) extraction from ground rosemary leaves (A). CA content in the final purified (washed) extracts (B), and mass yield (C) obtained by using 1:1 or 1:2 betaine:sorbitol deep eutectics containing 20 or 50% of water at 60° C. for 0.5 hour. The plant:solvent weight ratios were 1:8 (denoted 8 M), or 1:10 (denoted 10 M) and the precipitation was obtained using four volumes of water at room temperature.

FIG. 16. Recovery rate of carnosic acid (CA) extraction from ground rosemary leaves (A). CA content in the final purified (washed) extracts (B), and mass yield (C) obtained by using 1.1 sorbitol:levulinic acid deep eutectics containing 20 or 30% of water at room temperature (RT) for 0.5 hour. The plant:solvent weight ratio was 1:10 (denoted 10 M) and the precipitation was obtained using four volumes of water at room temperature.

FIG. 17. Recovery rate of carnosic acid (CA) extraction from ground rosemary leaves (A), CA content in the final purified (washed) extracts (B), and mass yield (C) obtained by using a 1:2 betaine:proline deep eutectics containing 30% of water at room temperature (RT) for 0.5, 1, or 2 hours. The plant:solvent weight ratio was 1:10 (denoted 10 M) and the precipitation was obtained using four volumes of water at room temperature.

FIG. 18. Recovery rate of carnosic acid (CA) extraction from ground rosemary leaves (A), CA content in the final purified (washed) extracts (B), and mass yield (C) obtained by using a 1:2 betaine:proline deep eutectics containing 30% of water at 60° C. for 0.5 or 2 hours. The plant:solvent weight ratio was 1:10 (denoted 10 M) and the precipitation was obtained using four volumes of water at room temperature.

FIG. 19. Recovery rate of carnosic acid (CA) extraction from ground rosemary leaves (A), CA content in the final purified (washed) extracts (B), and mass yield (C) obtained by using 1:1, 1:2, 2:1, or 3:1 proline:glucose deep eutectics containing 20 or 30% of water at 60° C. or room temperature (RT) for 0.5, 1, or 2 hours. The plant:solvent weight ratio was 1:10 (denoted 10 M) and the precipitation was obtained using four volumes of water at room temperature.

FIG. 20. Recovery rate of carnosic acid (CA) extraction from ground rosemary leaves (A), CA content in the final purified (washed) extracts (B), and mass yield (C) obtained by using 1:1, 1:2, 2:1, and 3:1 betaine:glucose deep eutectics containing 20, 30, or 50% of water at 60° C. for 0.5, 1, or 2 hours. The plant:solvent weight ratio was 1:10 (denoted 10 M) and the precipitation was obtained using four volumes of water at room temperature.

FIG. 21. Recovery rate of carnosic acid (CA) extraction from ground rosemary leaves (A), CA content in the final purified (washed) extracts (B), and mass yield (C) obtained by using a 1:2 betaine:pyruvic acid deep eutectics containing 25% of water at room temperature (RT) for 0.5 hour. The plant:solvent weight ratios were 1:8 (denoted 8 M), 1.10 (denoted 10 M), or 1:15 (denoted 15 M) and the precipitation was obtained using four volumes of water at room temperature.

FIG. 22. Recovery rate of carnosic acid (CA) extraction from ground rosemary leaves (A), CA content in the final crude (non-washed) or purified (washed) extracts (B), and mass yield (C) obtained by using a 2:1 urea:choline chloride deep eutectics free of exogenously added water, a 1:1 glycerol:lactic acid deep eutectics containing 20% of water, a 1:1 glycerol:levulinic acid deep eutectics containing 20% of water, a 1:1 sorbitol:lactic acid deep eutectics containing 20% of water, a 1:1 xylitol:levulinic acid deep eutectics containing 20% of water, a 1:2 glucose:levulinic acid deep eutectics containing 30% of water, a 1:1 proline:levulinic acid deep eutectics containing 20% of water, and a 1:1 lysine:levulinic acid deep eutectics containing 20% of water. Extractions have been performed at 60° C. or room temperature (RT) for 5 or 1 hour. The plant:solvent weight ratio was 1:10 (denoted 10 M) for all solvents and the precipitation was obtained using 4or 9 volumes of water at room temperature.

FIG. 23. Recovery rate of carnosic acid (CA) extraction from ground rosemary leaves using a 2:1 urea:betaine HCl deep eutectics free of exogenously added water, betaine:glycerol deep eutectics at various molar ratio (1:1, 1:2, or 2:1) and containing 10, 20, or 30% of water, 1:1 malic acid:choline chloride deep eutectics containing 20 or 30% of water, glycerol:choline chloride deep eutectics at various molar ratio (1:1, 1:2, or 2:1) and containing 0, 10, or 20% of water, a 1:1 glycerol:sorbitol deep eutectics containing 20% of water, a 1:2 lysine:levulinic acid deep eutectics containing 20% of water, and a 1:1 betaine:proline deep eutectics containing 30% water. All extractions have been performed at room temperature (RT) for 0.5 or 1 hour with a piant:solvent weight ratio of 1:10 (denoted 10 M).

FIG. 24. Antioxidant activity measured by the Rancimat assay of crude (non-washed) and purified (washed) extracts of rosemary obtained by using urea:betaine deep eutectics.

FIG. 25. Antioxidant activity measured by the Rancimat assay of purified (washed) extracts of rosemary obtained by using betaine:levulinic acid deep eutectics.

FIG. 26. Linear positive correlation between the protection factor measured using the Rancimat assay and the carnosic acid content in the final extract (%).

FIG. 27. Effect of the content of salt (NaCl) in water on the carnosic acid (CA) hydrosolubility expressed as the CA content in the supernatant.

FIG. 28. Active recovery rate (A), content in the final extracts (B) and mass yield (C) obtained after extraction for 30 min of ground turmeric roots using different DES and temperatures (room temperature, RT or 60° C.). Solvents used were 1:1 sorbitol:levulinic acid containing 20% of water, 1:1 proline:levulinic acid containing 20% of water, 1:2 betaine:glycerol containing 30% of water, 1:2 betaine:levulinic acid containing 25% of water, and 1:2 betaine:lactic acid containing 10% of water. For all solvents, the plant:solvent weight ratio was 1:10 and the precipitation was obtained using four volumes of water at room temperature.

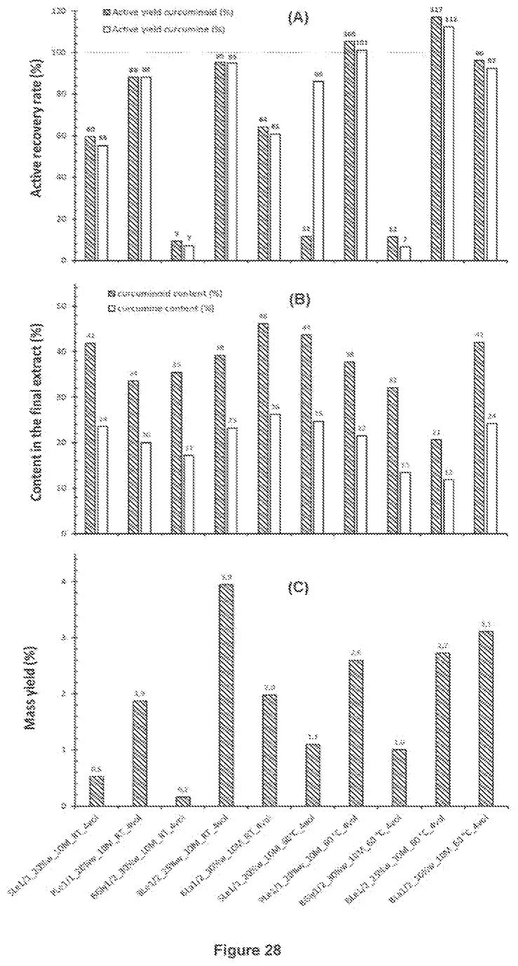

FIG. 29. Hesperidin recovery rate during extraction at room temperature for 30 min of ground orange peel using different DES. Solvents used were 1:1 sorbitol:levulinic acid containing 20% of water, 1:1 proline:levulinic acid containing 20% of water, 1:2 betaine:glycerol containing 30 of water, 1:2 betaine:levulinic acid containing 25% of water, and 1:2 betaine:lactic acid containing 10% of water. For all solvents, the plant:solvent weight ratio was 1:10.

### EXAMPLES

The present invention will be further described by reference to the following non-limiting examples.

Material and Methods

Solubilization of the Dye ‘Red Disperse 13’

Urea (≥98%, U5378, Sigma) and betaine (≥99%, 61962, Sigma) were of analytical grade. Care was taken to avoid any moisture by storing urea and betaine in a desiccator with silica. For choline chloride such caution was insufficient to avoid moisture and it was recrystallized with ethanol and stored in a desiccator with P₂O₅ as desiccant.

The desired amounts of betaine and/or urea powders were dissolved in distilled water or phosphate buffer solution (PBS, 67 mM) at pH 7.0 to form a 100 mL solution.

The concentration of urea in the urea:betaine mixtures in water or PBS ranged from 0.025 to 8.3 M.

The concentrations of pure urea solution in water or PBS ranged from 0.025 to 10.1 M, the latter value corresponding to the saturation level of urea.

Finally, the concentrations of pure betaine in water or PBS ranged from 0.025 to 5.4 M, this latter corresponding to the saturation level of betaine.

The solutions of urea and/or betaine in PBS were adjusted to provide a pH of 7.0 using concentrated HCl. The volume of added HCl was taken into account when calculating the final molar concentration of betaine and/or urea.

10 mL of the osmolyte solution was added to a vial containing 5 mg red disperse 13 in excess (final concentration: 364827, Sigma), then magnetically stirred (500 rpm) for 16 hours and filtrated (0.45 μm). The concentration of red disperse 13 in the filtrate is measured at 525 nm (Shimadzu UV-1800, Japan).

### EXAMPLE

### Example 1

### Solubilization Properties of Betaine:Urea Mixtures for Disperse Red 13 in Non-Buffered Aqueous Media

Betaine is a trimethylated form of glycine discovered for the first time in sugar beet juice. It is en abundant natural resource which can play the role of a hydrogen-bond acceptor through the two oxygen atoms of the carboxylate group (COO). Urea is known as a strong hydrogen-bond donor through the primary amine groups.

In this Example, anhydrous urea:betaine (U:B) mixtures at different molar ratios were first prepared, then solubilized in distilled water at different concentrations ranging from 1.5 g/L to the saturation level which was 607, 813, 985, 957, 795, 741, and 633 g/L for the U:B ratios 1:0, 3:1, 2:1, 1:1, 1:2, 1:3, and 0:1, respectively.

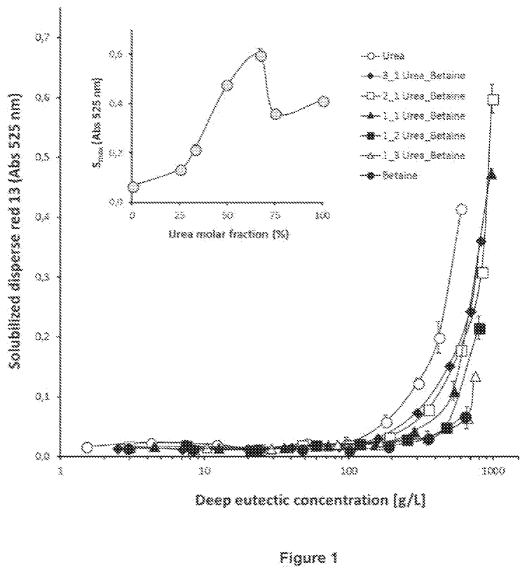

These liquid mixtures were mixed with an excess of disperse red 13 used as a hydrophobic probe to screen the solubilization properties of the solvents. It can be observed from FIG. 1 that the disperse red 13 absorbance increases exponentially 525 nm approx.) with increasing the deep eutectic concentration in water. This clearly demonstrates that the eutectic composition (1:2, betaine:urea) and the others (called peritectic mixtures instead of eutectic mixtures) behave as hydrotropes in water.

The MHC decreases as follows: 1:3 U:B>1:2 U:B>1:1 U:B>3:1 U:B>urea.

More importantly, the highest Sₘₐₓ (the maximal solubilization capacity of a given solute by a hydrotrope) was observed for the 1:2 betaine:urea ratio, which corresponds to the eutectic composition of the binary system. This is best observed in the FIG. 1 insert which plots Sₘₐₓ as a function of the urea molar fraction to betaine (%).

This means that, among all mixtures tested in this Example, the solvent with the highest capacity to solubilize the disperse red 13 was a 2:1 U:B solution at concentration of 985 g/L of water (saturation level or Cₘₐₓ).

In this example, taking into account that there is no plateau in the hydrotropy of urea and U:B mixtures, the maximal achievable concentrations (saturation level) of deep eutectics in water (Cₘₐₓ, x-axis, FIG. 1) correspond to the maximal achievable solubilizations of red disperse 13 (Sₘₐₓ, y-axis, FIG. 1). In other words, the Cₘₐₓ is the hydrotrope concentration enabling to reach the Sₘₐₓ for a given solute. Here, the higher the concentration in deep eutectics, the higher the concentratton of solubilized disperse red 13 (colligative effect). The fact that the optimized Sₘₐₓ is critically observed for the eutectic composition is an unprecedented finding.

Furthermore, although urea alone displays a hydrotropic behavior, this is not the case of betaine alone which increased the disperse red 13 too modestly to be qualified as such. The addition of urea to betaine in specific proportions (the eutectic composition, i.e. here 2:1 UB) thus lead to a strong synergistic effect of the corresponding mixture for solubilizing disperse red 13.

### Example 2

### Solubilization Properties of Betaine:Urea Mixtures for Disperse Red 13 in Buffered and pH 7-Adjusted Aqueous Media

To verify that the criticality of the U:B molar ratio is not simply due to a pH effect (pH varies depending on the ratio between urea and betaine and also on their concentration in the aqueous solution), a second series of experiments was conducted by solubilizing disperse red 13 in the same U:B mixtures but in a pH=7 phosphate buffer.

The high deep eutectic concentrations required to adjust the pH to 7 with a concentrated HCl solution because the buffering effect of the buffer was not always sufficient to counter the betaine-induced increase of pH. Added HCl volumes were less than 1% of the total volume in all tested mixtures, so the concentrations were not corrected.

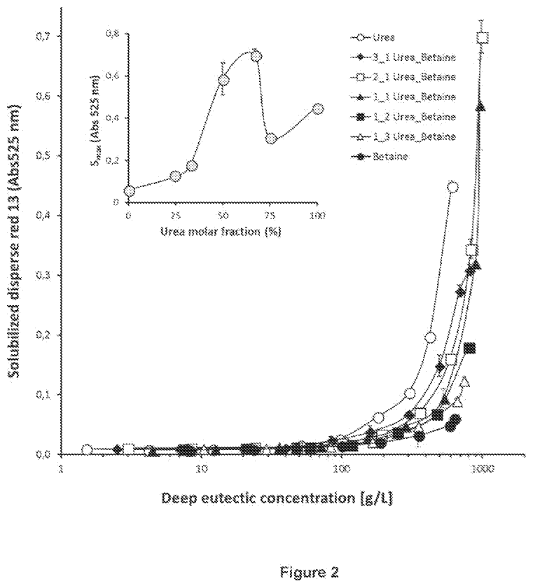

The results obtained were close to those seen in Example 1 with distillated water (uncontrolled pH) demonstrating that the deep eutectic hydrotopy is not primarily determined by the pH, but is rather controlled by the critical molar ratio between both components (FIG. 2).

Further experiments were thus conducted in distillated water. As previously observed, the eutectic composition (2:1 UB) at the saturation level in water offer the best conditions to solubilize the disperse red 13 dye. The exact same synergy as in Example 1 is also observed at pH 7 between urea and betaine.

### Example 3

### Solubilization Properties of Choline Chloride Urea Mixtures for Disperse Red 13 in Non-Buffered Aqueous Media

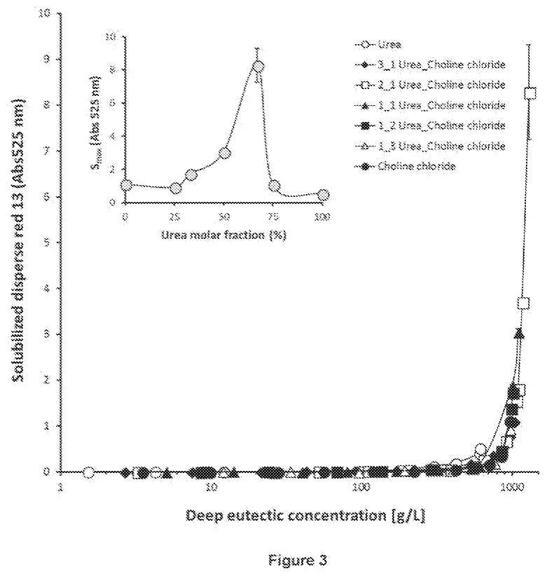

Choline chloride (ChCl) is a methylamine salt which can be either extracted from biomass or readily synthesized from fossil reserves through a very high atom economy process. In combination with hydrogen bond donors such as urea at a molar ratio of 2:1, urea:ChCl (UC); ChCl could produce a deep eutectic solvent that is liquid at 12 ° C. (FIG. 3).

### Example 4

### Solubilization Properties of Betaine:Urea Mixtures for Carnosic Acid in Non-Buffered Aqueous Media

In this Example, anhydrous urea:betaine (U:B) mixtures at different molar ratios were first prepared, then solubilized in distilled water at the saturation level; i.e. 607, 813, 985, 957, 795, 741, and 633 g/L for the U:B ratios 1:0. 3:1. 2:1, 1:1, 1:2, 1:3, and 0:1, respectively. These liquid mixture were mixed with an excess of carnosic acid, a diterpene antioxidant used as a hydrophobic probe to screen the solubilization properties of the different solvents.

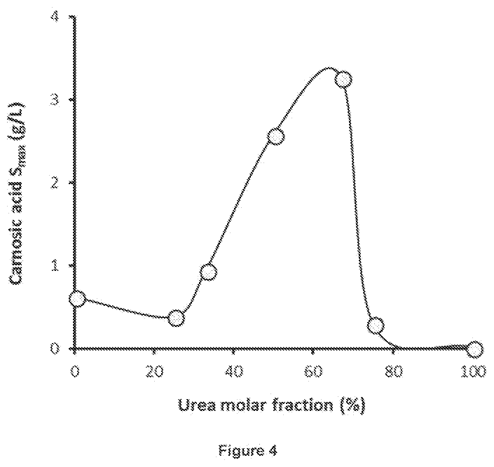

It can be observed from FIG. 4 that the highest carnosic acid Sₘₐₓ (the maximal solubilization capacity of carnosic acid by a deep eutectics) was observed for the 1:2 betaine:urea ratio, which corresponds to the eutectic composition of the binary system. This means that, among all mixtures tested in this Example, the solvent with the highest capacity to solubilize carnosic acid is a 2:1 U:B solution at concentration of 985 g/L of water (saturation level or Cₘₐₓ). Furthermore, a net synergy was obtained for the 2:1 U:B mixture.

### Example 5

### Solubilization Properties of Betaine Urea Mixtures for Hesperidin in Non-Buffered Aqueous Media

In this Example, anhydrous urea:betaine (U:B) mixtures at different molar ratios were first prepared, then solubilized in distilled water at the saturation level, i.e. 607, 813, 985, 957, 795, 741, and 633 g/L for the U:B ratios 1:0, 3:1, 2:1, 1:1, 1:2, 1:3, and 0:1, respectively.

These liquid mixtures were mixed with an excess of hesperidin, a flavonoid bioactive used as a hydrophobic probe to screen the solubilization properties of the different solvents.

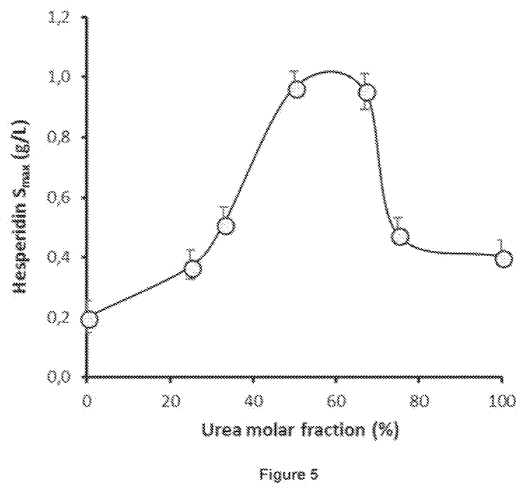

It can be observed from FIG. 5 that the highest hesperidin Sₘₐₓ (the maximal solubilization capacity of hesperidin by a deep eutectics) was observed for both the 1:2 and the 1:1 betaine:urea ratio, which corresponds to a composition nearby, or exactly at, the eutectic composition of the binary system.

This means that, among all mixtures tested in this Example, the solvents with the highest capacity to solubilize hesperidin are solutions with (i) a 2:1 U:B solution at concentration of 985 g/L of water and (ii) a 1:1 U:B solution at concentration of 957 g/L of water (saturation level or Cₘₐₓ in both cases). Furthermore, a net synergy was obtained for these two solvents compared to saturated solutions of pure betaine or pure urea.

### Example 6

### Solubilization Properties of Betaine:Urea Mixtures for Curcumin in Non-Buffered Aqueous Media

In this Example, anhydrous urea:betaine (U:B) mixtures at different molar ratios were first prepared, then solubilized in distilled water at the saturation level, i.e. 607, 813, 985, 957, 795, 741, and 633 g/L for the U:B ratios 1:0, 3:1, 2:1, 1:1, 1:2, 1:3, and 0:1, respectively. These liquid mixtures were mixed with an excess of curcumin, a phenolic bioactive and pigment used as a hydrophobic probe to screen the solubilization properties of the different solvents.

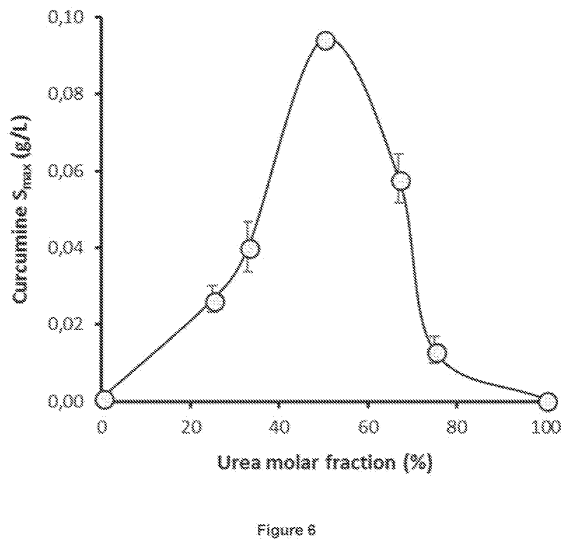

It can be observed from FIG. 6 that the highest curcumin Sₘₐₓ (the maximal solubilization capacity of curcumin by a deep eutectics) was observed for the 1:1 urea:betaine ratio, which corresponds to a composition nearby the eutectic composition of the binary system. This means that, among all mixtures tested in this Example, the solvents with me highest capacity to solubilize hesperidin is a 1:1 U:B solution at concentration of 957 g/L of water (saturation level or Cₘₐₓ). Furthermore, a net synergy was obtained for this solvent compared to saturated solutions of pure betaine or pure urea. In fact, all other combinations also exhibit a synergistic effect regarding their solubilization properties.

### Example 7

### Extraction of Rosemary Using Urea:Betaine Deep Eutectics, Cake Removal by Centrifugation then Filtration, Separation of the Extract from the Deep Eutectics Using Water as an Anti-Solvent, and Recovery of the Crude Extract by Centrifugation

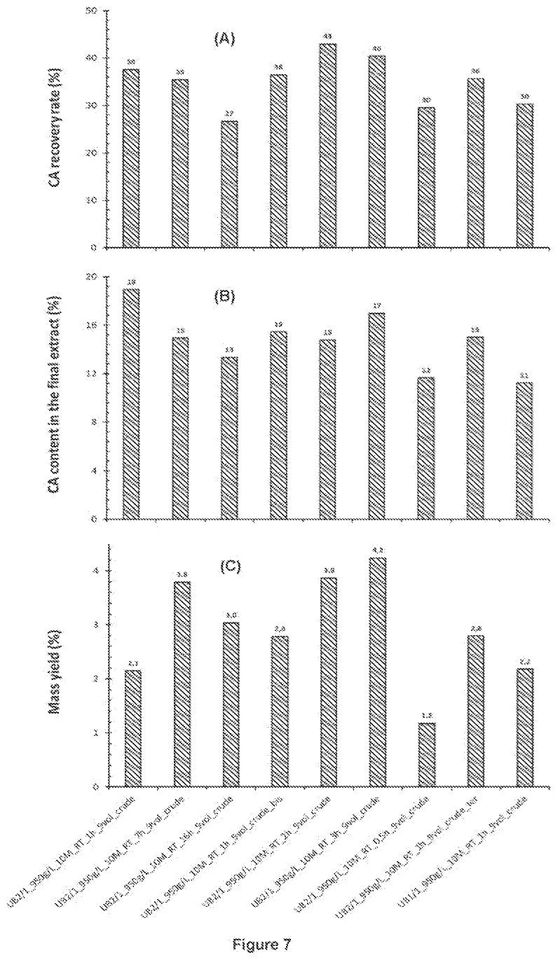

Homogeneous aqueous solutions of 2:1 urea:betaine (UB2/1_950 g/L) and 1.1 urea:betaine (UB1/1_950 g/L) mixtures both near the saturation level (950 g/L) were prepared. Twenty grams of ground rosemary leaves (containing 2.77% carnosic acid) were incubated at room temperature with 200 mL of this deep eutectic solution (plany:solvent: 1:10) for 0.5, 1, 2, 3, 7, or 16 hours and the enriched solution was separated from the plant by centrifugation followed by a filtration. The filtrate was analyzed by HPLC and the recovery rate was found to be comprised between 27 and 43% (FIG. 7a). Then, the filtrate was diluted by 9 volumes of water. This step of using water as an anti-solvent to precipitate the active components that will then constitute the crude extract is essential to separate most of the deep eutectics from the extract. The resulting precipitate was magnetically stirred for 15 minutes and left to settle for another 15 minutes. The precipitate was recovered by centrifugation and dried overnight at 45° C. in a vacuum oven. Finally, dried pellets (hereafter referred to as ‘crude extract’) was collected. They contained between 11 and 19% of carnosic acid (HPLC quantification) (FIG. 7b). The final mass yield was between 1.2 and 4.2% (FIG. 7c).

### Example 8

### Extraction of Rosemary Using Urea:Betaine Deep Eutectics, Cake Removal by Centrifugation then Filtration, Separation of the Extract from the Deep Eutectics Using Water as an Anti-Solvent, Recovery of the Crude Extract by Centrifugation, and Removal of Residual Deep Eutectics by a Washing Procedure with Water

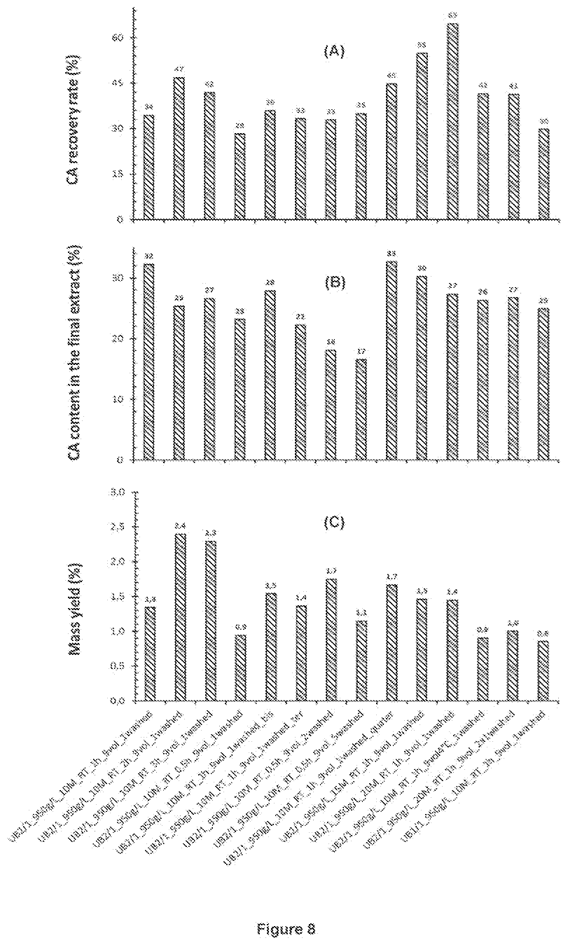

Here the process followed in Example 7 was reproduced with an additional washing procedure at the end. Homogeneous aqueous solutions of 2:1 urea:betaine (UB2/1_950 g/L) and 1:1 urea:betaine (UB1/1_950 g/L) mixtures both near the saturation level (950 g/L) were prepared. Twenty grams of ground rosemary leaves (containing 2.77% carnosic acid) were incubated at room temperature with 200 mL of this deep eutectic solution (plant:solvent 1:10) for 0.5, 1, 2, or 3 hours and the enriched solution was separated from the plant by centrifugation followed by a filtration. The filtrate was analyzed by HPLC and the recovery rate was found to be composed between 28 and 65% (FIG. 8a). Then, the filtrate was diluted by 9 volumes of water. This step of using water as an anti-solvent to precipitate the active components that will then constitute the crude extract is essential to separate most of the deep eutectics from the extract. The resulting precipitate was magnetically stirred for 15 minutes and left to settle for another 15 minutes.

Then, an additional washing procedure using 200 mL of water was applied to remove the residual deep eutectics from the extract (hereafter referred to as ‘solvent-free eutectic extract’ or ‘washed extract’) and further increase the carnosic acid concentration. The solvent-free eutectic extract that has been dried overnight at 45° C. in a vacuum oven finally contained between 17 and 33 of carnosic acid (HPLC quantification) ((FIG. 8b), which represent an important improvement of the carnosic acid content compared to the process without the washing procedure described in Example 7. The final mass yield concomitantly decreased with values ranging from 0.8 to 2.4% (FIG. 8c), which is still well acceptable regarding industrial standards.

### Example 9

### Chemical Characterization of the Rosemary Extracts Obtained in Examples 7 (Crude Extract) and 8 (Purified Extract) Using a 2:1 Urea:Betaine Mixture Near the Saturation Level (950 g/L) at Room Temperature for 2 Hour

Table 1 shows some of the identified compounds found in a eutectic rosemary extract before (crude extract corresponding to sample ‘UB2/1_950 g/L_10M_RT_2h_10vol_crude’ depicted in FIG. 7) and after (purified extract corresponding to sample ‘UB2/1_950 g/L_10M_RT_2h_10vol_1washed’ depicted in FIG. 8) the application of the washing procedure. The sign + means presence, and the sign − means absence.

TABLE 1
Identified compounds in eutectic rosemary extracts before (crude extract)
and after (purified extract) the application of the washing procedure

| Identified compounds in the | | Chemical | Crude | Purified |
|---|---|---|---|---|
| eutectic extracts | Formula | family | extract | extract |
| Gluconic acid | C₆H₁₂O₇ | Organic acid | + | − |
| Malic acid | C₄H₆O₅ | Organic acid | + | − |
| Tartaric acid | C₄H₆O₄ | Organic acid | + | − |
| Salycilic acid | C₇H₆O₃ | Phenolic acid | + | − |
| Caffeic acid | C₉H₈O₄ | Phenolic acid | + | − |
| Nepitrin | C₂₂H₂₂O₁₂ | Flavonoid | + | − |
| Rosmarinic acid | C₁₈H₁₆O₈ | Phenolic acid | + | − |
| Luteolin glucuronide | C₂₁H₁₈O₁₂ | Flavonoid | + | − |
| Luteolin 3-O-(o-acetyl) D | C₂₃H₂₀O₁₃ | Flavonoid | + | + |
| glucuronide isomer I | | | | |
| Luteolin 3-O-(o-acetyl) D | C₂₃H₂₀O₁₃ | Flavonoid | + | + |
| glucuronide isomer II | | | | |
| Luteolin 3-O-(o-acetyl) D | C₂₃H₂₀O₁₃ | Flavonoid | + | − |
| glucuronide isomer III | | | | |
| Scutellarein | C₁₅H₁₀O₆ | Flavonoid | + | − |
| Nepetin/isorhamnetin | C₁₆H₁₂O₇ | Flavonoid | + | + |
| Diosmetin | C₁₆H₁₂O₆ | Flavonoid | + | + |
| Hispidulin | C₁₆H₁₂O₆ | Flavonoid | + | + |
| Cirsimaritin | C₁₇H₁₄O₆ | Flavonoid | + | + |
| Apigenin | C₁₅H₁₀O₅ | Flavonoid | + | + |
| Dimethoxycoumarin | C₁₁H₁₀O₄ | Coumarin | + | + |
| Rhamnazin/dimethylquercetin | C₁₇H₁₄O₇ | Flavonoid | + | + |
| Rosmanol | C₂₀H₂₆O₅ | Phenolic | + | + |
| | | diterpene | | |
| Cirsimaritin isomer | C₁₇H₁₄O₆ | Flavonoid | + | + |
| Epiisorosmanol | C₂₀H₂₆O₅ | Phenolic | + | + |
| | | diterpene | | |
| Epirosmanol | C₂₀H₂₆O₅ | Phenolic | + | + |
| | | diterpene | | |
| Hydroxycryptotanshinone | C₁₉H₂₀O₄ | Diterpenoid | − | + |
| Genkwanin | C₁₆H₁₂O₅ | Flavonoid | + | + |
| Epirasmanol isomer | C₂₀H₂₆O₅ | Phenolic | − | + |
| | | diterpene | | |
| Gingerol | C₁₇H₂₆O₄ | Phenolic lipid | − | + |
| Rosmadial | C₂₀H₂₄O₅ | Phenolic | + | + |
| | | diterpene | | |
| Epirosmanol methyl ether | C₂₁H₂₈O₅ | Phenolic | + | + |
| | | diterpene | | |
| Ubiquinol | C₁₈H₂₈O₄ | Phenolic lipid | − | + |
| para-Miltioic acid | C₁₉H₂₄O₅ | Diterpenoid | − | + |
| Epirosmanol methyl ether | C₂₁H₂₈O₅ | Phenolic | + | + |
| | | diterpene | | |
| Carnosol | C₂₀H₂₆O₄ | Phenolic | + | + |
| | | diterpene | | |
| Carnosol isomer | C₂₀H₂₆O₄ | Phenolic | + | + |
| | | diterpene | | |
| Rosmadial isomer | C₂₀H₂₄O₅ | Phenolic | + | + |
| | | diterpene | | |
| Rosmaridiphenol | C₂₀H₂₈O₃ | Phenolic | + | + |
| | | diterpene | | |
| Carnosic acid | C₂₀H₂₈O₄ | Phenolic | + | + |
| | | diterpene | | |
| 12-Methoxycarnosic acid | C₂₁H₃₀O₄ | Phenolic | + | + |
| | | diterpene | | |
| 5,6,7,10-Tetrahydro-7- | C₁₉H₂₆O₃ | Phenolic | − | + |
| hydroxyrosmariquinone | | diterpene | | |
| Shogaol | C₂₀H₃₀O₃ | Phenolic lipid | + | + |
| Ursolic acid | C₃₀H₄₈O₃ | Triterpene | + | + |

### Example 10

### Extraction of Rosemary Using Betain:Levulinic Acid DES, Cake Removal by Filtration, Separation of the Extract from the Deep Eutectics Using Water as an Anti-Solvent, and Recovery of the Crude Extract by Centrifugation

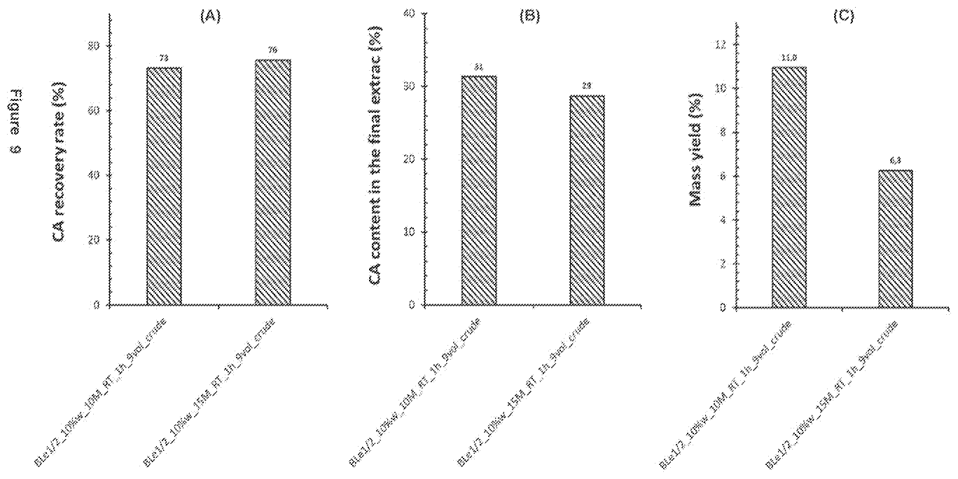

A homogeneous solution of 1:2 betaine:levulinic acid_mixture containing 10% of water (BLe1/2_10% w) was prepared. Twenty grams of ground rosemary leaves (containing 2.77% carnosic acid) were incubated at room temperature with 200 mL (plant:solvent:1:10) or 300 mL (plant:solvent 1:15) of this deep eutectic solution for one hour and the enriched solution was separated from the plant by filtration. The filtrate was analyzed by HPLC and the recovery rate was found to be comprised between 73 and 76% (FIG. 9a). Then, the filtrate was diluted by 9 volumes of water. This step of using water as an anti-solvent to precipitate the active components that will then constitute the crude extract is essential to separate most of the deep eutectics from the extract. The resulting precipitate was magnetically stirred for 15 minutes and left to settle for another 15 minutes. The precipitate was recovered by centrifugation and freeze-dried overnight. Finally, dried pellets (hereafter referred to as ‘crude extract’) was collected. They contained between 29 and 31% of carnosic acid (HPLC quantification) (FIG. 9b). The final mass yield was between 6.3 and 11% (FIG. 9c).

### Example 11

### Extraction of Rosemary Using Betaine:Levulinic Acid DES, Cake Removal by Filtration, Separation of the Extract from the Deep Eutectics Using Water as an Anti-Solvent, Recovery of the Crude Extract by Centrifugation, and Removal of Residual Deep Eutectics by a Washing Procedure with Water

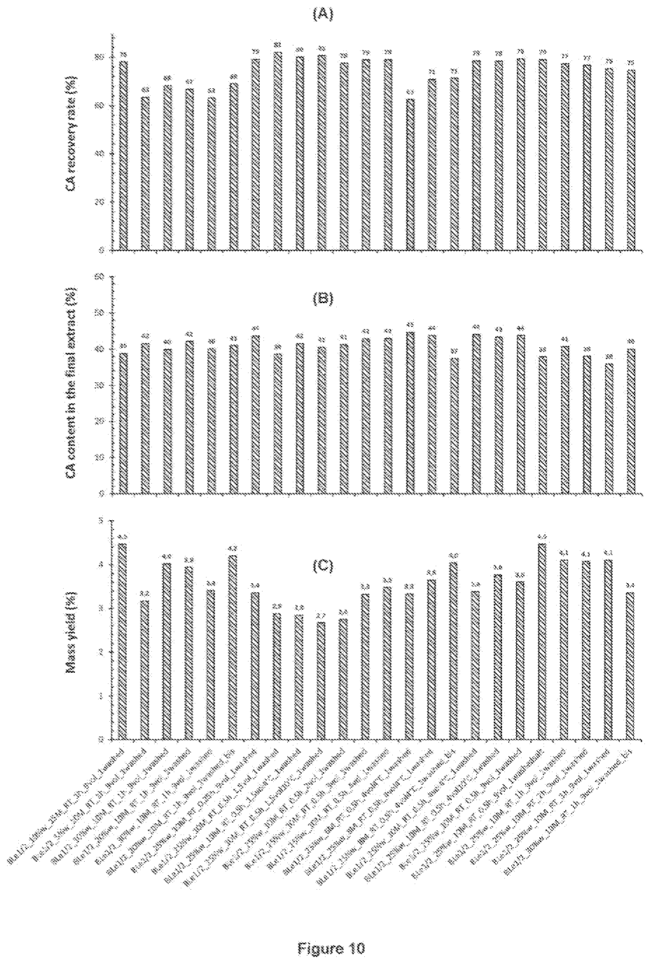

Here the process followed in Example 10 was reproduced with an additional washing procedure at the end. Homogeneous solutions of 1:2 betaine:levulinic acid mixtures containing 5% (BLel1/2_5%w), 10% (BLe1/2_10% w), 20% (BLe1/2_20% w), 25% (BLe1/2_25% w), and 30% (BLe1/2_30% w) of water were prepared. Twenty grams of ground rosemary leaves (containing 2.77% carnosic acid) were incubated at room temperature with 120 mL (plant:solvent: 1:6), 160 mL (plant:solvent: 1:8), 200 mL (plant:solvent: 1:10), or 300 mL (plant:solvent: 1:15) of this deep eutectic solution for 0.26, 0.5, 1, 2, or 3 hours and the enriched solution was separated from the plant by filtration. The filtrate was analyzed by HPLC and the recovery rate was found to be comprised between 63 and 82% (FIG. 10a). Then, the filtrate was diluted by 1.5, 2, 3, 4, and 9 volumes of water at 4 or 10° C. or at room temperature. This step of using water as an anti-solvent to precipitate the active components that will then constitute the crude extract is essential to separate most of the deep eutectics from the extract. The resulting precipitate was magnetically stirred for 15 minutes and left to settle for another 15 minutes.

Then, an additional washing procedure using the same volume of water as the volume or solvent used for the extraction (120, 160, 200, or 300 mL) was applied to remove the residual deep eutectics from the extract and further increase the carnosic acid concentration. The solvent-free eutectic extract that has been freeze-dried overnight finally contained between 36 and 45% of carnosic acid (HPLC quantification). (FIG. 10b), which represents an important improvement of the carnosic acid content compared to the process without the washing procedure described in Example 10. The final mass yield concomitantly decreased with values ranging from 2.7 to 4.5% (FIG. 10c), which is still well acceptable regarding industrial standards.

### Example 12

### Extraction of Rosemary Using Betaine:Lactic Acid DES, Cake Removal by Filtration, Separation of the Extract from the Deep Eutectics Using Water as an Anti-Solvent and Recovery of the Crude Extract by Centrifugation

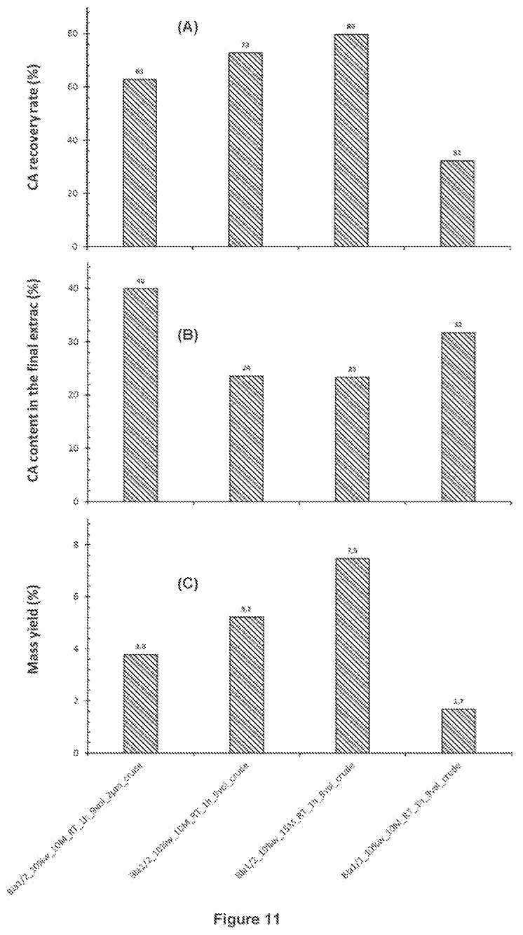

Homogeneous solutions of 1:2 betaine:lactic acid (BLa1/2_10% w) and 1:1 betaine:latic acid (BLa1/1_10% w) mixtures each containing 10% of water were prepared. Twenty grams of ground rosemary leaves (containing 2.77% carnosic acid) were incubated at room temperature with 200 mL (plant:solvent 1:10) or 300 mL (plant:solvent: 1:15) of these deep eutectic solutions for one hour and the enriched solution was separated from the plant by filtration. The filtrate was analyzed by HPLC and the recovery rate was found to be comprised between 32 and 80% (FIG. 11a). Then, the filtrate was diluted by 9 volumes of water. This step of using water as an anti-solvent to precipitate the active components that will then constitute the crude extract is essential to separate most of the deep eutectics from the extract. The resulting precipitate was magnetically stirred for 15 minutes and left to settle for another 15 minutes. The precipitate was recovered by centrifugation and freeze-dried overnight. Finally, dried pellets were collected. They contained between 23 and 40% of carnosic acid (HPLC quantification) (FIG. 11b). The final mass yield was between 1.7 and 7.5% (FIG. 11c).

### Example 13

### Extraction of Rosemary Using Betame:Lactic Acid DES, Cake Removal by Filtration, Separation of the Extract from the Deep Eutectics Using Water as an Anti-Solvent Recovery of the Crude Extract by Centrifugation, and Removal of Residual Deep Eutectics by a Washing Procedure with Water

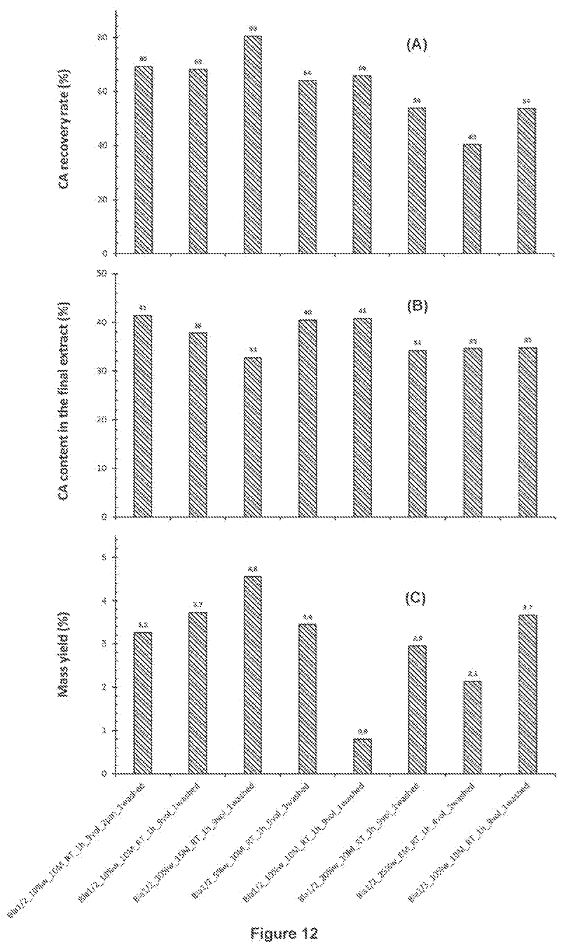

Here the process followed in Example 12 was reproduced with an additional washing procedure at the end. Homogeneous solutions of 1:2 betaine:lactic acid mixtures containing 5% (BLa1/2_5% w), 10% (BLa1/2_10% w), 20% (BLa1/2_20% w), and 25% (BLa1/2_25% w) % of water and a solution of 1:3 betaine:lactic acid mixture containing 10% of water (BLa1/3_10% w) were prepared. Twenty grams of ground rosemary leaves (containing 2.77% carnosic acid) were incubated at room temperature with 160 mL (plant:solvent 1:8), 200 mL (plant:solvent 1:10), or 300 mL (plant:solvent 1:15) of this deep eutectic solution for one hour and the enriched solution was separated from the plant by filtration. The filtrate was analyzed by HPLC and the recovery rate was found to be compnsed between 40 and 80% (FIG. 12a). Then, the filtrate was diluted by 4 or 9 volumes of water at room temperature. This step of using water as an anti-solvent to precipitate the active components that will then constitute the crude extract is essential to separate most of the deep eutectics from the extract. The resulting precipitate was magnetically stirred for 15 minutes and left to settle for another 15 minutes. Then, an additional washing procedure using the same volume of water as the volume of solvent used for the extraction (160, 200, or 300 ml) of water was applied to remove the residual deep eutectics from the extract and further increase the carnosic acid concentration. The solvent-free eutectic extract that has been freeze-dried overnight finally contained between 33 and 41% of carnosic acid (HPLC quantification) (FIG. 12b). The final mass yield ranged from 0.8 to 4.6% (FIG. 12c), which is still well acceptable regarding industrial standards.

### Example 14

### Extraction of Rosemary Using Betaine:Glycerol DES, Cake Removal by Filtration, Separation of the Extract from the Deep Eutectics Using Water as an Anti-Solvent, and Recovery of the Crude Extract by Centrifugation

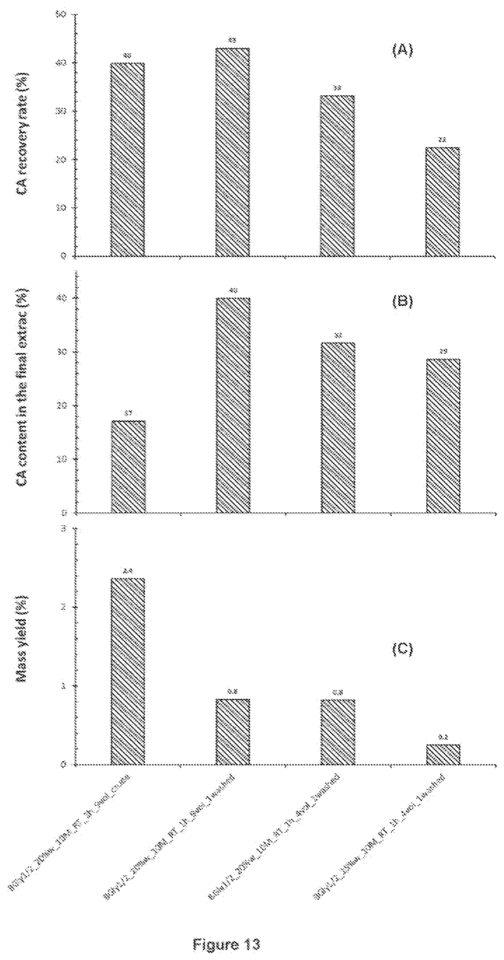

Homogeneous solutions of 1:2 betaine:glycerol mixture containing 20% of water (BGly1/2_20% w) were prepared. Twenty grams of ground rosemary leaves (containing 2.77% carnosic acid) were incubated at room temperature with 200 mL (10 M) of this deep eutectic solutions for one hour and the enriched solution was separated from the plant by filtration. The filtrate was analyzed by HPLC and the recovery rate was found to be 40% (FIG. 13a, sample ‘BGly1/2_20% w_10M_RT_1h_9vol_crude’). Then, the filtrate was diluted by 9 volumes of water. This step of using water as an anti-solvent to precipitate the active components that will then constitute the crude extract is essential to separate most of the deep eutectics from the extract. The resulting precipitate was magnetically stirred for 15 minutes and left to settle for another 15 minutes. The precipitate was recovered by centrifugation and freeze-dried overnight. Finally, dried pellets were collected. They contained 17% of carnosic acid (HPLC quantification) (FIG. 13b). The final mass yield was 2.4% (FIG. 13c).

### Example 15

### Extraction of Rosemary Using Betame:Glycerol DES, Cake Removal by Filtration, Separation of the Extract from the Deep Eutectics Using Water as an Anti-Solvent Recovery of the Crude Extract by Centrifugation, and Removal of Residual Deep Eutectics by a Washing Procedure with Water

Here the process followed in Example 14 was reproduced with an additional washing procedure at the end. Homogeneous solutions of 1:2 betaine:glycerol mixtures containing 20% (BGly1/2_20% w) and 25% (BGly1/2_25% w) of water were prepared. Twenty grams of ground rosemary leaves (containing 2.77% carnosic acid) were incubated at room temperature with 200 ml of this deep eutectic solution for one hour and the enriched solution was separated from the plant by filtration. The filtrate was analyzed by HPLC and the recovery rate was found to be comprised between 22 and 43 % (FIG. 13a). Then, the filtrate was diluted by 4 or 9 volumes of water at room temperature. This step of using water as an anti-solvent to precipitate the active components that will then constitute the crude extract is essential to separate most of the deep eutectics from the extract. The resulting precipitate was magnetically stirred for 15 minutes and left to settle for another 15 minutes.

Then, an additional washing procedure using 200 mL of water was applied to remove the residual deep eutectics from the extract and further increase the carnosic acid concentration. The solvent-free eutectic extract that has been freeze-dried overnight finally contained between 29 and 40% of carnosic acid (HPLC quantification) (FIG. 13b). The final mass yield ranged from 0.2 to 0.8% (FIG. 13c), which is still well acceptable regarding industrial standards.

### Example 16

### Extraction of Rosemary Using Betaine:Sorbitol DES at Room Temperature, Cake Removal by Filtration, Separation of the Extract from the Deep Eutectics Using Water as an Anti-Solvent, Recovery of the Crude Extract by Centrifugation, and Removal of Residual Deep Eutectics by a Washing Procedure with Water

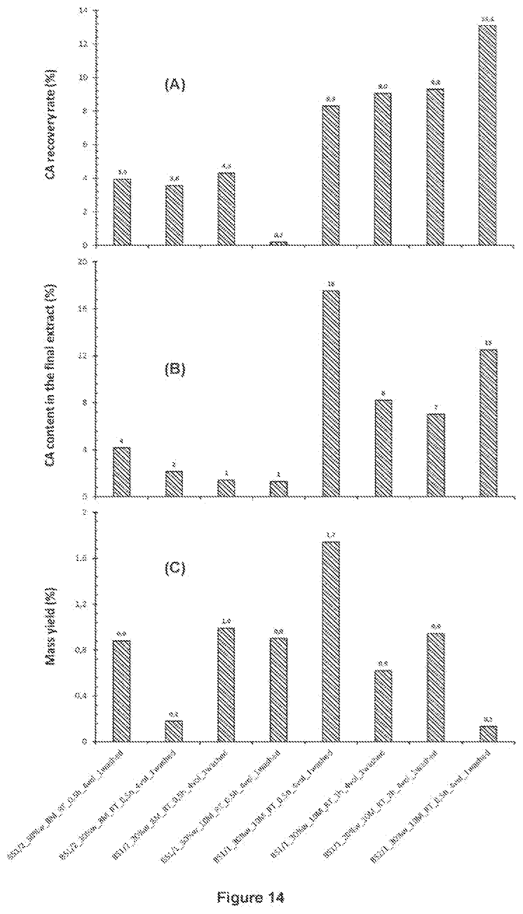

Homogeneous solutions of 1:2 betaine:sorbitol mixtures containing 20% (BS1/2_20% w), 30% (BS1/2_30% w) and 50% (BS1/2_50% w) of water were prepared as well as solutions of 1:1 betaine:sorbitol mixtures containing 20% (BS1/1_20% w), 30% (BS1/1_30% w) and 50% (BS1/1_50% w), and 2:1 betaine:sorbitol mixtures containing 30% (BS2/1_30% w). Twenty grams of ground rosemary leaves (containing 2.77% carnosic acid) were incubated at room temperature with 160 mL (plant:solvent: 1:8), 200 mL (plant:solvent: 1:10); and 300 mL (plant:solvent: 1.15) of this deep eutectic solution for 0.5, 1, and 2 hour and the enriched solution was separated from the plant by filtration. The filtrate was analyzed by HPLC and the recovery rate was found to be comprised between 3.9 and 13.1% (FIG. 14a). Then, the filtrate was diluted by 4 volumes of water at room temperature. This step of using water as an anti-solvent to precipitate the active components that will then constitute the crude extract is essential to separate most of the deep eutectics from the extract. The resulting precipitate was magnetically stirred for 15 minutes and left to settle for another 15 minutes.

Then, an additional washing procedure using the same volume of water as the volume of solvent used for the extraction (160, 200, or 300 mL) was applied to remove the residual deep euterctics from the extract and further increase the camosic acid concentration. The solvent-free eutectic extract that has been freeze-dried overnight finally contained between 1 and 18% of carnosic acid (HPLC quantification) (FIG. 14b). The final mass yield ranged from 0.1 to 1.7% (FIG. 14c), which is still well acceptable regarding industrial standards.

### Example 17

### Extraction of Rosemary Using Betaine:Sorbitol DES at 60° C. Cake Removal by Filtration, Separation of the Extract from the Deep Eutectics Using Water as an Anti-Solvent, Recovery of the Crude Extract by Centrifugation, and Removal of Residual Deep Eutectics by a Washing Procedure with Water

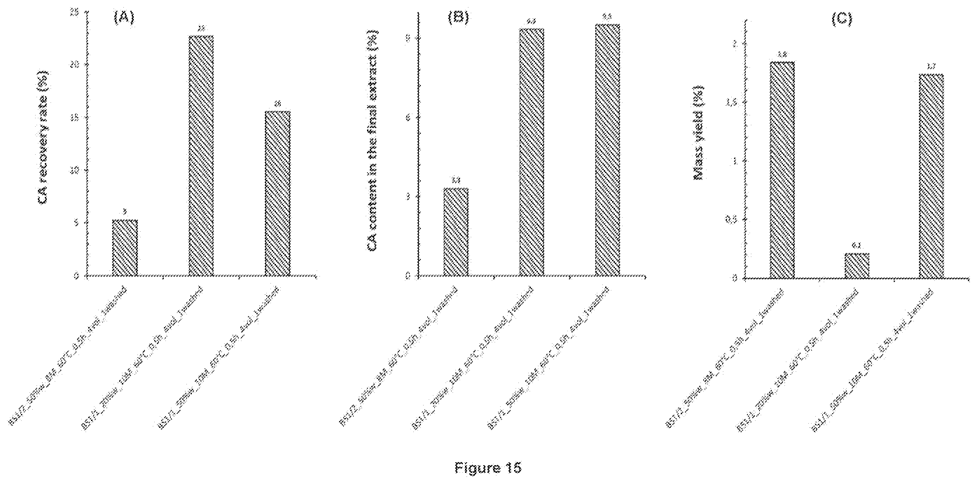

Homogeneous solutions of 1:2 betaine:sorbitol mixtures containing 50% of water (BS1/2_50% w) and solutions of 1:1 betaine:sorbitol mixtures containing 20% (BS1/1_20% w), and 50% (BS1/1_50% w) of water were prepared. Twenty grams of ground rosemary leaves (containing 2.77% carnosic acid) were incubated at 60° C. with 160 mL (plant:solvent 1:8) or 200 mL (plant:solvent 1:10) of this deep eutectic solution for 0.5, 1, or 2 hours and the enriched solution was separated from the plant by filtration. The filtrate was analyzed by HPLC and the recovery rate was found to be comprised between 3.9 and 13.1% (FIG. 15a). Then, the filtrate was diluted by 4 volumes of water at room temperature. This step of using water as an anti-solvent to precipitate the active components that will then constitute the crude extract is essential to separate most of the deep, eutectics from the extract. The resulting precipitate was magnetically stirred for 15 minutes and left to settle for another 15 minutes.

Then, an additional washing procedure using the same volume of water as the volume of solvent used for the extraction (160 or 200 mL) was applied to remove the residual deep eutectics from the extract and further increase the carnosic acid concentration. The solvent-free eutectic extract that has been freeze-dried overnight finally contained between 1 and 18% of carnosic acid (HPLC quantification) (FIG. 15b). The final mass yield ranged from 0.1 to 1.7% (FIG. 15c), which is still well acceptable regarding industrial standards.

### Example 18

### Extraction of Rosemary Using Sorbitol:Levulinic Acid DES, Cake Removal by Filtration, Separation of the Extract from the Deep Eutectics Using Water as an Anti-Solvent, Recovery of the Crude Extract by Centrifugation, and Removal of Residual Deep Eutectics by a Washing Procedure with Water

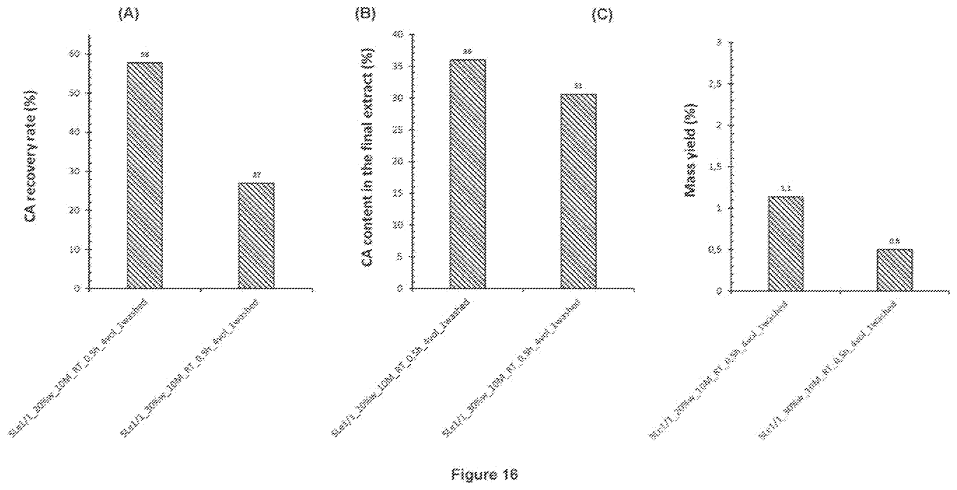

Homogenous solutions of 1:1 sorbitol:levulinic acid mixtures containing 20% (SLe1/1_20% w) or 30% (SLe1/1_30% w) of water were prepared. Twenty grams of ground rosemary leaves (containing 2.77% carnosic acid) were incubated at room temperature with 200 mL (plant solvent: 1:10) of this deep eutectic solution for 0.5 hour, and the enriched solution was separated from the plant by filtration. The filtrate was analyzed by HPLC and the recovery rate was found to be 58 and 27%, respectively (FIG. 16a) Then, the filtrate was diluted by 4 volumes of water at room temperature. This step of using water as an anti-solvent to precipitate the active components that will then constitute the crude extract is essential to separate most of the deep eutectics from the extract. The resulting precipitate was magnetically stirred for 15 minutes and left to settle for another 15 minutes.

Then, an additional washing procedure using 200 mL of water was applied to remove the residual deep eutectics from the extract and further increase the carnosic acid concentration. The solvent-free eutectic extract that has been freeze-dried overnight finally contained 36% of carnosic acid for SLe1/1_20% w and 31% for SLe1/1_30% w (HPLC quantification) (FIG. 16b). The final mass yield was found to be 1.1 and 0.5, respectively (FIG. 16c), which is still well acceptable regarding industrial standards.

### Example 19

### Extraction of Rosemary Using Betaine:Proline DES at Room Temperature, Cake Removal by Filtration, Separation of the Extract from the Deep Eutectics Using Water as an Anti-Solvent, Recovery of the Crude Extract by Centrifugation, and Removal of Residual Deep Eutectics by a Washing Procedure with Water

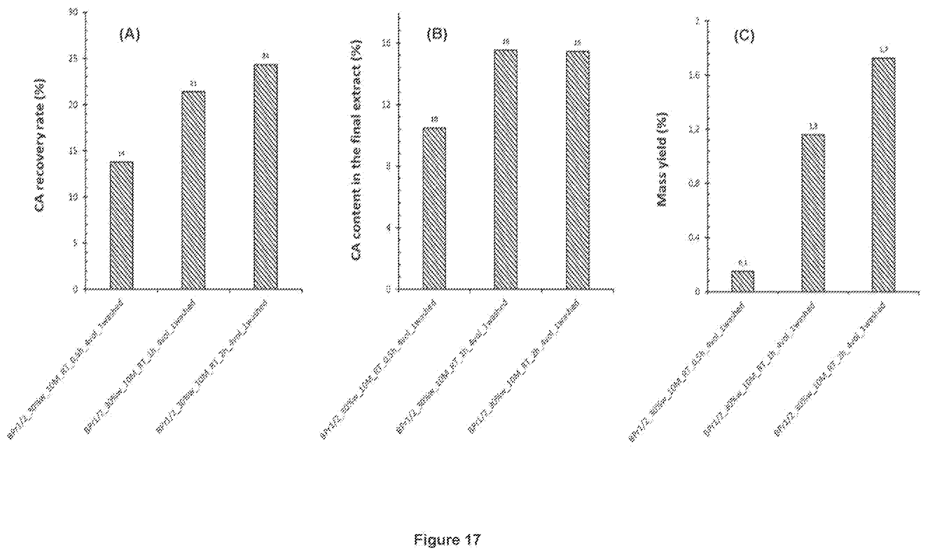

A homogenous solution of 1:2 betaine:proline mixture containing 30% of water was prepared. Twenty grams of ground rosemary leaves (containing 2.77% carnosic acid) were incubated at room temperature with 200 mL (plant:solvent: 1:10) of this deep eutectic solution for 0.5, 1, or 2 hours, and the enriched solution was separated from the plant by filtration. The filtrate was analyzed by HPLC and the recovery rate was found to be between 14 and 24% (FIG. 17a). Then, the filtrate was diluted by 4 volumes of water at room temperature. This step of using water as an anti-solvent to precipitate the active components that will then constitute the crude extract is essential to separate most of the deep eutectics from the extract. The resulting precipitate was magnetically stirred for 15 minutes and left to settle for another 15 minutes.

Then, an additional washing procedure using 200 mL of water was applied to remove the residual deep eutectics from the extract and further increase the carnosic acid concentration. The solvent-free eutectic extract that has been freeze-dried overnight finally contained between 10 and 16% of carnosic acid (HPLC quantification) (FIG. 17b). The final mass yield was found to be between 0.1 and 1.7 (FIG. 17c), which is still well acceptable regarding industrial standards.

### Example 20

### Extracton of Rosemary Using Betaine:Proline DES at 60° C. Cake Removal by Filtration, Separation of the Extract from the Deep Eutectics Using Water as an Anti-Solvent Recovery of the Crude Extract by Centrifugation, and Removal of Residual Deep Eutectics by a Washing Procedure with Water

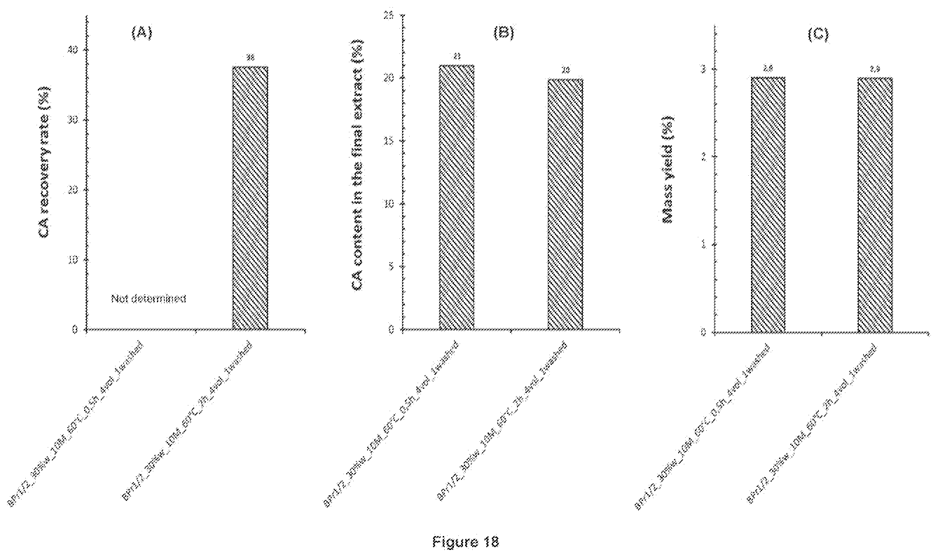

A homogenous solution of 1:2 betaine:proline mixture containing 30% of water was prepared. Twenty grams of ground rosemary leaves (containing 2.77% carnosic acid) were incubated at 60° C. with 200 mL (plant:solvent; 1:10) of this deep eutectic solution for 0.5, or 2 hours, and the enriched solution was separated from the plant by filtration. The filtrate was analyzed by HPLC and the recovery rate was found to be 38% for the extraction performed after 2 hours (FIG. 18a). Unfortunately, the recovery rate has not been evaluated for the extraction performed after 0.5 hour.

Then, the filtrate was diluted by 4 volumes of water at room temperature. This step of using water as an anti-solvent to precipitate the active components that will then constitute the crude extract is essential to separate most of the deep eutectics from the extract. The resulting precipitate was magnetically stirred for 15 minutes and left to settle for another 15 minutes.

Then, an additional washing procedure using 200 mL of water was applied to remove the residual deep eutectics from the extract and further increase the carnosic acid concentration. The solvent-free eutectic extract that has been freeze-dried overnight finally contained 21% (0.5 h) and 20% (2 h) of carnosic acid HPLC quantification) (FIG. 18b). The final mass yield was found to be 2.9% in both cases (FIG. 18c).

### Example 21

### Extraction of Rosemary Using Proline:Glucose DES, Cake Removal by Filtration, Separation of the Extract from the Deep Eutectics Using Water as an Anti-Solvent, Recovery of the Crude Extract by Centrifugation, and Removal of Residual Deep Eutectics by a Washing Procedure with Water

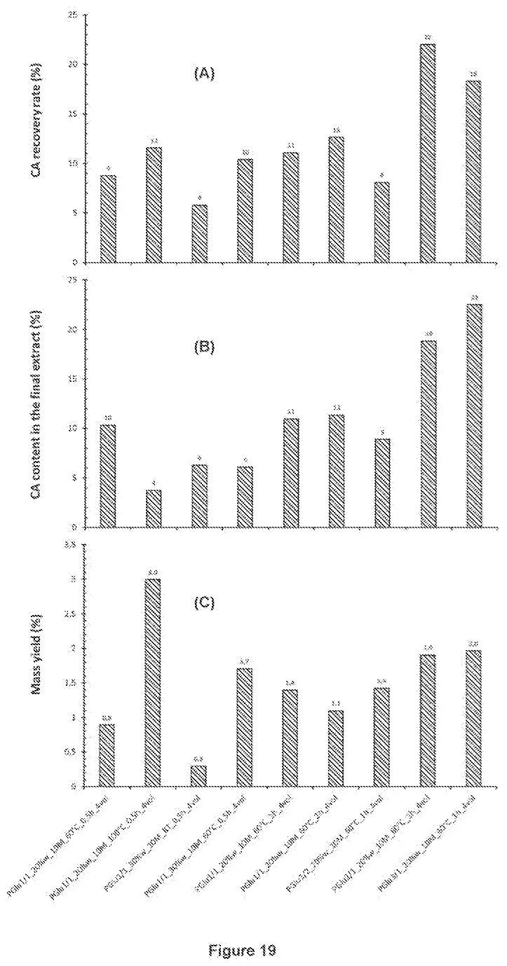

Homogenous solutions of 1:1 proline:glucose mixtures containing 20% (PGlu1/1_20% w) or 30% (PGlu1/1_30% w) of water were prepared as well as 1:2 proline:glucose (PGlu1/2_20% w), 2:1 proline:glucose (PGlu2/1_20% w), and 3:1 proline:glucose (PGlu3/1_20% w) mixtures each containing 20% of water. Twenty grams of ground rosemary leaves (containing 2.77% carnosic acid) were incubated at room temperature, 60° C, or 100° C. with 200 mL (plant:solvent: 1:10) of this deep eutectic solution for 0.5, 1, or 2 hours, and the enriched solution was separated from the plant by filtration. The filtrate was analyzed by HPLC and the recovery rate was found to be between 6 and 22% (FIG. 19a). Then, the filtrate was diluted by 4 volumes of water at room temperature. This step of using water as an anti-solvent to precipitate the active components that will then constitute the crude extract is essential to separate most of the deep eutectics from the extract. The resulting precipitate was magnetically stirred for 15 minutes and left to settle for another 15 minutes.

Then, an additional washing procedure using 200 mL of water was applied to remove the residual deep eutectics from the extract and further increase the carnosic acid concentration. The solvent-free eutectic extract that has been freeze-dried overnight finally contained between 4 and 23% of carnosic acid (HPLC quantification) (FIG. 19b). The final mass yield was found to be between 0.3 and 3% (FIG. 19c), which is still well acceptable regarding industrial standards.

### Example 22

### Extraction of Rosemary Using Betaine:Glucose DES, Cake Removal by Filtration, Separation of the Extract from the Deep Eutectics Using Water as an Anti-Solvent Recovery of the Crude Extract by Centrifugation, and Removal of Residual Deep Eutectics by a Washing Procedure with Water

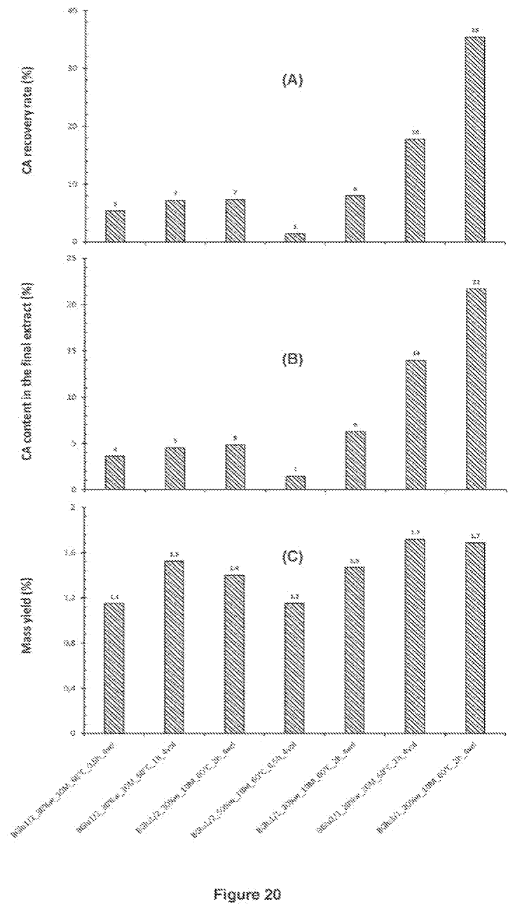

Homogenous solutions of 1:2 betaine:glucose mixtures containing 30% (BGlu1/2_30% w) or 50% (BGlu1/2_50% w) of water were prepared as well as 1:1 betaine:glucose (BGlu1/2_20% w), 2:1 betaine:glucose (BGlu2/1_20% w), and 3:1 betaine:glucose (BGlu3/1_20% w) mixtures each containing 20% of water. Twenty grams of ground rosemary leaves (containing 2.77% carnosic acid) were incubated at 60° C. with 200 mL (plant:solvent 1:10) of this deep eutectic solution for 0.5, 1, or 2 hours, and the enriched solution was separated from the plant by filtration. The filtrate was analyzed by HPLC and the recovery rate was found to be between 1 and 35% (FIG. 20a). Then, the filtrate was diluted by 4 volumes of water at room temperature. This step of using water as an anti-solvent to precipitate the active components that will then constitute the crude extract is essential to separate most of the deep eutectics from the extract. The resulting precipitate was magnetically stirred for 15 minutes and left to settle for another 15 minutes.

Then, an additional washing procedure using 200 mL of water was applied to remove the residual deep eutectics from the extract and further increase the carnosic acid concentration. The solvent-free eutectic extract that has been freeze-dried overnight finally contained between 1 and 22% of carnosic acid (HPLC quantification) (FIG. 20b). The final mass yield was found to be between 1.1 and 1.7% (FIG. 20c).

### Example 23

### Extraction of Rosemary Using Betaine:Pyruvic Acid DES, Cake Removal by Filtration, Separation of the Extract from the Deep Eutectics Using Water as an Anti-Solvent, Recovery of the Crude Extract by Centrifugation, and Removal of Residual Deep Eutectics by a Washing Procedure with Water

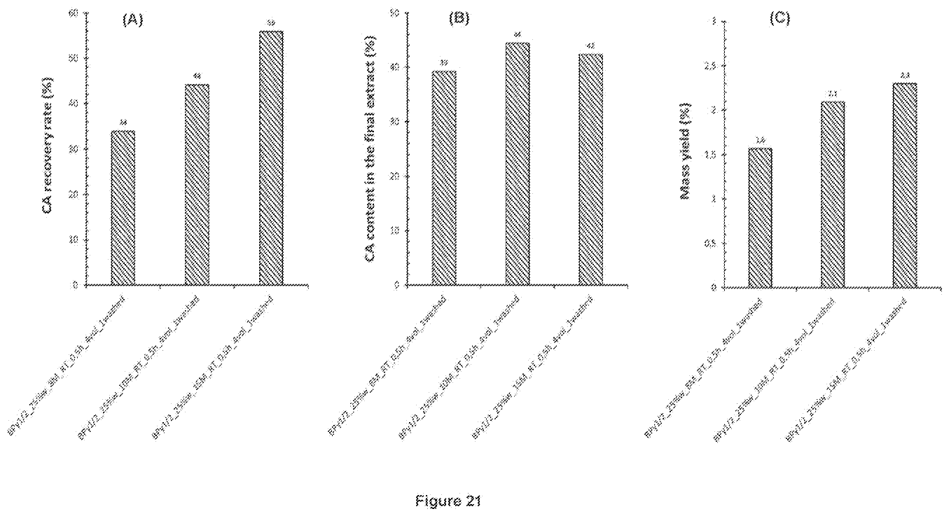

A homogenous solution of 1.2 betaine:pyruvic acid mixture containing 25% of water was prepared. Twenty grams of ground rosemary leaves (containing 2.77% carnosic acid) were incubated at room temperature with 160 mL (plant:solvent: 1:8), 200 mL (plant:solvent 1:10), or 300 mL (plant:solvent: 1:15) of this deep eutectic solution for 0.5 hour, and the enriched solution was separated from the plant by filtration. The filtrate was analyzed by HPLC and the recovery rate was found to be 34, 44, and 56%, respectively (FIG. 21a).

Then, the filtrate was diluted by 4 volumes of water at room temperature. This step of using water as an anti-solvent to precipitate the active components that will then constitute the crude extract is essential to separate most of the deep eutectics from the extract. The resulting precipitate was magnetically stirred for 15 minutes and left to settle for another 15 minutes.

Then, an additional washing procedure using the same volume of water as the volume of solvent used for the extraction (160, 200, or 300 mL) was applied to remove the residual deep eutectics from the extract and further increase the carnosic acid concentration. The solvent-free eutectic extract that has been freeze-dried overnight finally contained 39% (plant:solvent: 1:8), 44% (plant:solvent: 1:10), and 42% (plant:solvent: 1:15) of carnosic acid (HPLC quantification) (FG. 21b). The final mass yield was found to be 1.6, 2.1, and 2.3%, respectively (FIG. 18c).

### Example 24

### Extraction of Rosemary Using Urea:Choline chloride DES, Cake Removal by Centrifugation then Filtration, Separation of the Extract from the Deep Eutectics Using Water as an Anti-Solvent, and Recovery of the Crude Extract by Centrifugation

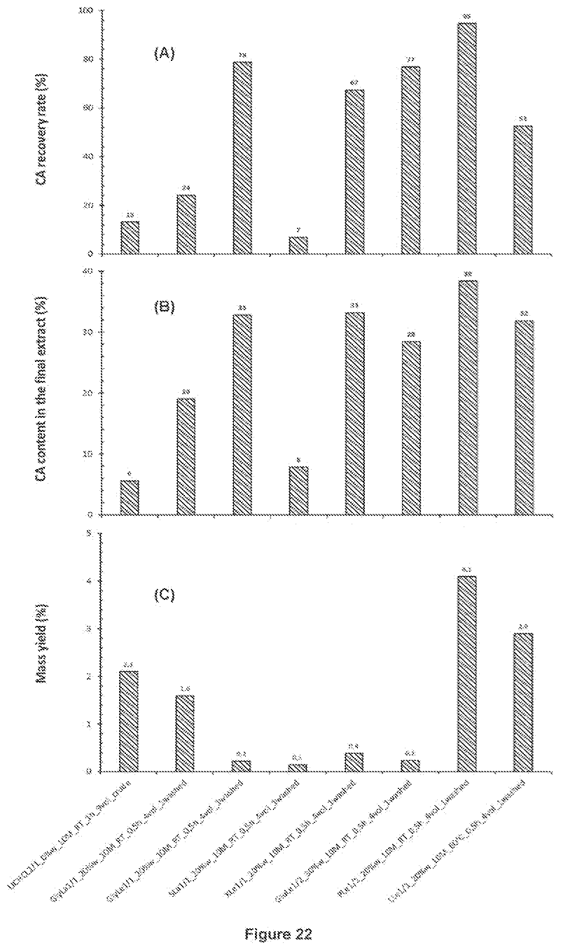

A homogeneous solution of a 2:1 urea:choline chloride neat mixture (UChCl2/1_0% w) was prepared. Twenty grams of ground rosemary leaves (containing 2.77% carnosic acid) were incubated at room temperature with 200 mL (plant:solvent: 1:10) of this deep eutectic solutions for one hour, and the enriched solution was separated from the plant by filtration. The filtrate was analyzed by HPLC and the recovery rate was found to be 13% (FIG. 22a, sample ‘UChCl2/1_0% w_10M_RT_1h_9vol_crude’). Then, the filtrate was diluted by 9 volumes of water. This step of using water as an anti-solvent to precipitate the active components that will then constitute the crude extract is essential to separate most of the deep eutectics from the extract. The resulting precipitate was magnetically stirred for 15 minutes and left to settle for another 15 minutes. The precipitate was recovered by centrifugation and freeze-dried overnight. Finally, dried pellets were collected. They contained 6% of carnosic acid (HPLC quantification) (FIG. 22b). The final mass yield was 2.1% (FIG. 22c).

### Example 25

### Extraction of Rosemary Using Glycerol:Lactic Acid DES, Cake Removal by Filtration, Separation of the Extract from the Deep Eutectics Using Water as an Anti-Solvent, Recovery of the Crude Extract by Centrifugation, and Removal of Residual Deep Eutectics by a Washing Procedure with Water

A homogeneous solution of a 1:1 glycerol:lactic acid mixture containing 20% of water (GlyLa1/1_20% w) was prepared. Twenty grams of ground rosemary leaves (containing 2.77% carnosic acid) were incubated at room temperature with 200 mL (plant:solvent:1:10) of this deep eutectic solution for 0.5 hour, and the enriched solution was separated from the plant by filtration. The filtrate was analyzed by HPLC and the recovery rate was found to be 24% (FIG. 22a). Then, the filtrate was diluted by 4 volumes of water at room temperature. This step of using water as an anti-solvent to precipitate the active components that will then constitute the crude extract is essential to separate most of the deep eutectics from the extract. The resulting precipitate was magnetically stirred for 15 minutes and left to settle for another 15 minutes.

Then, an additional washing procedure using 200 ml of water was applied to remove the residual deep eutectics from the extract and further increase the carnosic acid concentration. The solvent-free eutectic extract that has been freeze-dried overnight finally contained 19% of carnosic acid (HPLC quantification) (FIG. 22b). The final mass yield was found to be 1.6% (FIG. 22c).

### Example 26

### Extraction of Rosemary Using Glycerol:Levulinic Acid DES, Cake Removal by Filtration, Separation of the Extract from the Deep Eutectics Using Water as an Anti-Solvent, Recovery of the Crude Extract by Centrifugation, and Removal of Residual Deep Eutectics by a Washing Procedure with Water

A homogeneous solution of a 1:1 glycerol:levulinic acid mixture containing 20% of water (GlyLe1/1_20% w) was prepared. Twenty grams of ground rosemary leaves (containing 2.77% carnosic acid) were incubated at room temperature with 200 mL (plant:solvent 1.10) of this deep eutectic solution for 0.5 hour, and the enriched solution was separated from the plant by filtration. The filtrate was analyzed by HPLC and the recovery rate was found to be 79% (FIG. 22a). Then, the filtrate was diluted by 4 volumes of water at room temperature. This step of using water as an anti-solvent to precipitate the active components that will then constitute the crude extract is essential to separate most of the deep eutectics from the extract. The resulting precipitate was magnetically stirred for 15 minutes and left to settle for another 15 minutes.

Then, an additional washing procedure using 200 mL of water was applied to remove the residual deep eutectics from the extract and further increase the carnosic acid concentration. The solvent-free eutectic extract that has been freeze-dried overnight finally contained 33% of carnosic acid (HPLC quantification) (FIG. 22b). The final mass yield was found to be 0.2% (FIG. 22c).

### Example 21

### Extraction of Rosemary Using Sorbitol:Lactic Acid DES, Cake Removal by Filtration, Separation of the Extract from the Deep Eutectics Using Water as an Anti-Solvent, Recovery of the Crude Extract by Centrifugation, and Removal of Residual Deep Eutectics by a Washing Procedure with Water

A homogeneous solution of a 1:1 sorbitol:lactic add mixture containing 20% of water (SLa1/1_20% w) was prepared. Twenty grams of ground rosemary leaves (containing 2.77% carnosic acid) were incubated at room temperature with 200 mL (plant:solvent:1:10) of this deep eutectic solution for 0.5 hour, and the enriched solution was separated from the plant by filtration. The filtrate was analyzed by HPLC and the recovery rate was found to be 7% (FIG. 22a). Then, the filtrate was diluted by 4 volumes of water at room temperature. This step of using water as an anti-solvent to precipitate the active components that will then constitute the crude extract is essential to separate most of the deep eutectics from the extract. The resulting precipitate was magnetically stirred for 15 minutes and left to settle for another 15 minutes.

Then, an additional washing procedure using 200 mL of water was applied to remove the residual deep eutectics from the extract and further increase the carnosic acid concentration. The solvent-free eutectic extract that has been freeze-dried overnight finally contained 8% of carnosic acid (HPLC quantification) (FIG. 22b). The final mass yield was found to be 0.1% (FIG. 22c).

### Example 28

### Extraction of Rosemary Using Xylitol:Levulinic Acid DES, Cake Removal by Filtration, Separation of the Extract from the Deep Eutectics Using Water as an Anti-Solvent Recovery of the Crude Extract by Centrifugation, and Removal of Residual Deep Eutectics by a Washing Procedure with Water

A homogeneous solution of a 1:1 xylitol:levulinic acid mixture containing 20% of water (XLe1/1_20% w) was prepared. Twenty grams of ground rosemary leaves (containing 2.77% carnosic acid) were incubated at room temperature with 200 mL (plant solvent:1:10) of this deep eutectic solution for 0.5 hour, and the enriched solution was separated from the plant by filtration. The filtrate was analyzed by HPLC and the recovery rate was found to be 67% (FIG. 22a). Then, the filtrate was diluted by 4 volumes of water at room temperature. This step of using water as an anti-solvent to precipitate the active components that will then constitute the crude extract is essential to separate most of the deep eutectics from the extract. The resulting precipitate was magnetically stirred for 15 minutes and left to settle for another 15 minutes.

Then, an additional washing procedure using 200 mL of water was applied to remove the residual deep eutectics from the extract and further increase the carnosic acid concentration. The solvent-free eutectic extract that has been freeze-dried overnight finally contained 33% of carnosic acid (HPLC quantification) (FIG. 22b). The final mass yield was found to be 0.4% (FIG. 22c).

### Example 29

### Extraction of Rosemary Using Glucose:Levulinic Acid DES, Cake Removal by Filtration, Separation of the Extract from the Deep Eutectics Using Water as an Anti-Solvent Recovery of the Crude Extract by Centrifugation, and Removal of Residual Deep Eutectics by a Washing Procedure with Water

A homogeneous solution of a 1:2 glucose:levulinic acid mixture containing 30% of water (GluLe1/2_30% w) was prepared. Twenty grams of ground rosemary leaves (containing 2.77% carnosic acid) were incubated at room temperature with 200 mL (plant:solvent:1:10) of this deep eutectic solution for 0.5 hour, and the enriched solution was separated from the plant by filtration. The filtrate was analyzed by HPLC and the recovery rate was found to be 77% (FIG. 22a). Then, the filtrate was diluted by 4 volumes of water at room temperature. This step of using water as an anti-solvent to precipitate the active components that will then constitute the crude extract is essential to separate most of the deep eutectics from the extract. The resulting precipitate was magnetically stirred for 15 minutes and left to settle for another 15 minutes.

Then, an additional washing procedure using 200 mL of water was applied to remove the residual deep eutectics from the extract and further increase the carnosic acid concentration. The solvent-free eutectic extract that has been freeze-dried overnight finally contained 28% of carnosic acid (HPLC quantification) (FIG. 22b). The final mass yield was found to be 0.2% (FIG. 22c).

### Example 30

### Extraction of Rosemary Using Proline:Levulinic Acid DES, Cake Removal by Filtration, Separation of the Extract from the Deep Eutectics Using Water as an Anti-Solvent, Recovery of the Crude Extract by Centrifugation, and Removal of Residual Deep Eutectics by a Washing Procedure with Water

A homogeneous solution of a 1:1 proline:levulinic acid mixture containing 20% of water (PLe1/1_20% w) was prepared. Twenty grams of ground rosemary leaves (containing 2.77% carnosic acid) were incubated at room temperature with 200 mL (plant:solvent:1.10) of this deep eutectic solution for 0.5 hour, and the enriched solution was separated from the plant by filtration. The filtrate was analyzed by HPLC and the recovery rate was found to be 95% (FIG. 22a). Then, the filtrate was diluted by 4 volumes of water at room temperature. This step of using water as an anti-solvent to precipitate the active components that will then constitute the crude extract is essential to separate most of the deep eutectics from the extract. The resulting precipitate was magnetically stirred for 15 minutes and left to settle for another 15 minutes.

Then, an additional washing procedure using 200 mL of water was applied to remove the residual deep eutectics from the extract and further increase the carnosic acid concentration. The solvent-free eutectic extract that has been freeze-dried overnight finally contained 38% of carnosic acid (HPLC quantification) (FIG. 22b). The final mass yield was found to be 4.1% (FIG. 22c).

### Example 31

### Extraction of Rosemary Using Lysine:Levulinic Acid DES, Cake Removal by Filtration, Separation of the Extract from the Deep Eutectics Using Water as an Anti-Solvent, Recovery of the Crude Extract by Centrifugation, and Removal of Residual Deep Eutectics by a Washing Procedure with Water

A homogeneous solution of a 1:1 lysine:levulinic acid mixture containing 20% of water (PLe1/1_20% w) was prepared. Twenty grams of ground rosemary leaves (containing 2.77% carnosic acid) were incubated at 60° C. with 200 mL (plant:solvent: 1:10) of this deep eutectic solution for 0.5 hour, and the enriched solution was separated from the plant by filtration. The filtrate was analyzed by HPLC and the recovery rate was found to be 53% (FIG. 22a). Then, the filtrate was diluted by 4 volumes of water at room temperature. This step of using water as an anti-solvent to precipitate the active components that will then constitute the crude extract is essential to separate most of the deep eutectics from the extract. The resulting precipitate was magnetically stirred for 15 minutes and left to settle for another 15 minutes.

Then, an additional washing procedure using 200 mL of water was applied to remove the residual deep eutectics from the extract and further increase the carnosic acid concentration. The solvent-free eutectic extract that has been freeze-dried overnight finally contained 32% of carnosic acid (HPLC quantification) (FIG. 22b). The final mass yield was found to be 2.9% (FIG. 22c).

### Example 32

### Extraction of Rosemary Using Urea:Betaine HCI DES, and Cake Removal by Centrifugation then Filtration

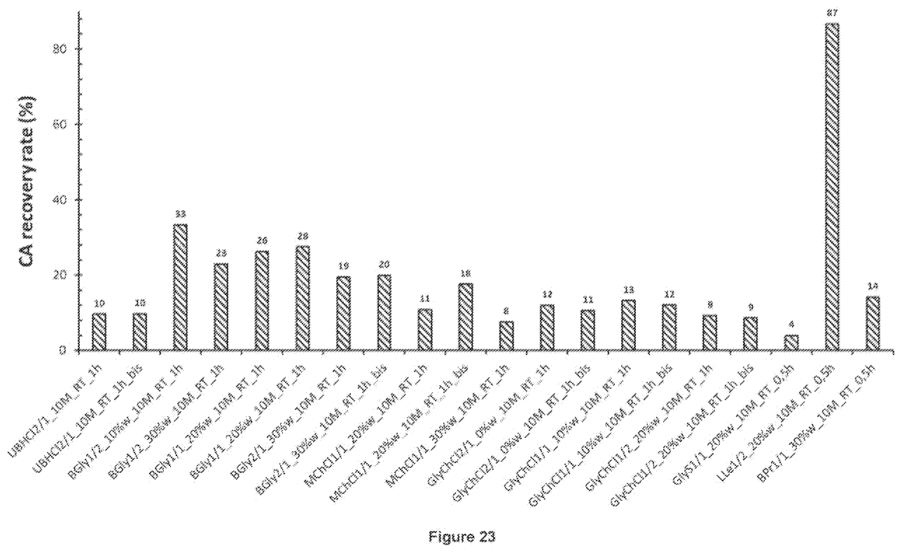

A homogeneous solution of a 2:1 urea:betaine HCl neat mixture (UBHCl2/1) was prepared. Twenty grams of ground rosemary leaves (containing 2.77% carnosic acid) were incubated at room temperature with 200 mL (plant:solvent: 1:10) of this deep eutectic solution for one hour, and the enriched solution was separated from the plant by centrifugation, then filtration. The filtrate was analyzed by HPLC and the recovery rate was found to be 10% (FIG. 23).

### Example 33

### Extraction of Rosemary Using Betaine:Glycerol DES, and Cake Removal by Filtration

Homogeneous solutions of 1:2 betaine:glycerol mixtures containing 10% (BGly1/2_10% w) and 30% (BGly1/2_30% w) of water was prepared as well as 1:1 betaine:glycerol mixtures containing 20% of water (BGly1/1_20% w), and 2:1 betaine:glycerol mixtures containing 30% of water (BGly2/1_30% w). Twenty grams of ground rosemary leaves (containing 2.77% carnosic acid) were incubated at room temperature with 200 mL (plant:solvent: 1:10) of this deep eutectic solution for one hour, and the enriched solution was separated from the plant by filtration. The filtrate was analyzed by HPLC and the recovery rate was found to be comprised between 19 and 33% (FIG. 23).

### Example 34

### Extraction of Rosemary Using Malic Acid:Choline Chloride DBS, and Cake Removal by Filtration

Homogeneous solutions of 1:1 malic acid:choline chloride mixtures containing 20% (MChCl1/1_20% w) and 30% (MChCl1/1_30% w) of water was prepared. Twenty grams of ground rosemary leaves (containing 2.77% carnosic acid) were incubated at room temperature with 200 mL (plant:solvent: 1.10) of this deep eutectic solution for one hour, and the enriched solution was separated from the plant by filtration. The filtrate was analyzed by HPLC and the recovery rate was found to be comprised between 8 and 18% (FIG. 23).

### Example 35

### Extraction of Rosemary Using Glycerol:Choline Chloride DES, and Cake Removal by Filtration

Homogeneous solutions of a 2:1 glycerol:choline chloride neat mixture (GlyChCl2/1_0% w), a 1:1 glycerol:choline chloride mixture containing 10% of water (GlyChCl1/1_10% w), and a 1:2 glycerol:choline chloride mixture containing 20% of water (GlyChCl1/2_20% w)_were prepared. Twenty grams of ground rosemary leaves (containing 2.77% carnosic acid) were incubated at room temperature with 200 mL (plant:solvent: 1:10) of this deep eutectic solution for one hour, and the enriched solution was separated from the plant by filtration. The filtrate was analyzed by HPLC and the recovery rate was found to be composed between 9 and 13% (FIG. 23).

### Example 36

### Extraction of Rosemary Using Glycerol:Sorbitol DES, and Cake Removal by Filtration

A homogeneous solution of a 1:1 gycerol:sorbitol mixture containing 20% of water (GlyS1/1_20% w) was prepared. Twenty grams of ground rosemary leaves (containing 2.77% carnosic acid) were incubated at room temperature with 200 mL (plant.solvent:1:10) of this deep eutectic solution for 0.5 hour, and the enriched solution was separated from the plant by filtration. The filtrate was analyzed by HPLC and the recovery rate was found to be 4% (FIG. 23).

### Example 37

### Extraction of Rosemary Using Lysine:Levulinic Acid DES, and Cake Removal by Filtration

A homogeneous solution of a 1:2 lysine:levulinic acid mixture containing 20% of water (LLe1/2_20% w) was prepared. Twenty grams of ground rosemary leaves (containing 2.77% carnosic acid) were incubated at room temperature with 200 mL (plant:solvent:1:10) of this deep eutectic solution for 0.5 hour, and the enriched solution was separated from the plant by filtration. The filtrate was analyzed by HPLC and the recovery rate was found to be 87% (FIG. 23).

### Example :38

### Extraction of Rosemary Using Betaine:Proline DES, and Cake Removal by Filtration

A homogeneous solution of a 1:1 betaine:proline mixture containing 30% of water (BPr1/1_30% w) was prepared. Twenty grams of ground rosemary leaves (containing 2.77% carnosic acid) were incubated at room temperature with 200 mL (plant:solvent:1:10) of this deep eutectic solution for 0.5 hour, and the enriched solution was separated from the plant by filtration. The filtrate was analyzed by HPLC and the recovery rate was found to be 14 (FIG. 23).

### Example 39

### Negative Controls Showing the Importance of the DES Combination for the Precipitation/Flocculation Step

To further verify that the process of the invention necessitates the combination of two molecules (hence the formation of a DES), such as betaine:levulinic acid far example, we tested some negative control for the precipitation step. Indeed, we reproduced the exact same process as for 1:2 betaine:levulinic acid containing 10% or 25% of water on the sole levulinic acid without any betaine added in the mixture (Table 2). Ground rosemary leaves were incubated in these mixtures at a plant:solvent weight ratio of 1:10 for one hour at room temperature. While the first step of extraction was successfully performed with carnosic acid recovery rates ranging from 84 to 93%, then it was not possible to recover any crude extract by filtration or centrifugation after we applied the standard precipitation step with the addition of water.

Then, we directly used pure levulinic acid without any water addition (nor betaine) and performed the extraction on ground rosemary leaves at a plant:solvent weight ratio of 1:10 for one hour at room temperature. Again, we were unable to collect the precipitate formed (if any) with the same procedure that the one currently applied on DES.

A last verification was done by using betaine alone containing 40% of water (near the saturation level in water) using the same extraction and precipitation procedure as for levulinic acid alone except that the extraction duration was 0.5 h. Although 9.2% of carnosic acid was extracted, again, the collect of the precipitate (if any) was unsuccessful.

TABLE 2
Negative controls used for the precipitation/flocculation step

| | | | | | CA | Vol. of | Quantity of extract |
|---|---|---|---|---|---|---|---|
| Studied | Water | Plant:solvent | | | recovery | water to | collected after |
| molecule | content | ratio | Time | | rate (%) | precipitate | precipitation (mg) |
| Levulinic | 10% | 1:10 | 1 | h | 84.5 | 4 | 0 |
| acid | | | | | | | |
| Levulinic | 10% | 1:10 | 1 | h | 87.0 | 4 | 0 |
| acid | | | | | | | |
| Levulinic | 25% | 1:10 | 1 | h | 92.7 | 4 | 0 |
| acid | | | | | | | |
| Levulinic | 0% | 1:10 | 1 | h | 97.9 | 4 | 0 |
| acid | | | | | | | |
| Betaine | 40% | 1:10 | 0.5 | h | 9.2 | 4 | 0 |

These negative controls demonstrate in the example of levulinic acid:betaine DES that the combination of the two compounds is absolutely required to obtain a crude or a purified rosemary extract, because no extract can be collected when either levulinic acid alone or betaine alone are used instead of the corresponding DES.

### Example 40

### Antioxidant Activity (Rancimat Method) of the Crude and Purified Eutectic Extracts of Rosemary Obtained with Urea:Betaine Deep Eutectics and Formulated in a Sunflower Oil

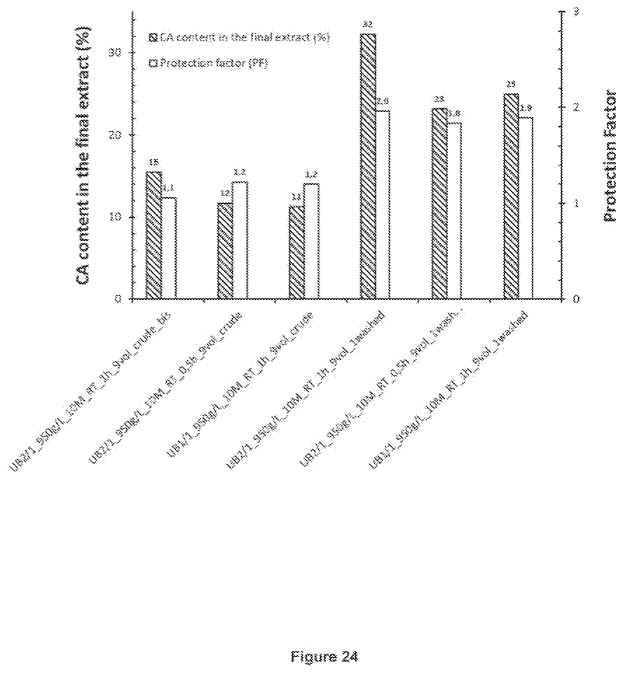

The crude (non-washed) and purified (washed) rosemary extracts obtain using the process of the invention described in Examples 7 and 8 with urea betaine deep eutectics were assessed for their antioxidant activity by the Rancimat method. Extracts were solubilized in sunflower oil at a final concentration of 1000 mg/kg, and were introduced in glass tubes in the Rancimat apparatus. The samples were heated at 110° C. and flushed with air at a flux of 10 L/h to accelerate the oxidation kinetics. The conductivity of the collected headspace allows to precisely evaluate the lag phase that passes until oxidation significantly increases (induction time). By dividing the induction time of the sample treated with a rosemary extract by that of an untreated sunflower oil control, we can determine the protection factor (PF). A PF of 1 shows that the extract is non-antioxidant, while a PF with a higher value demonstrates an antioxidant activity. Here we show that three different crude rosemary extracts obtained using 2:1 urea:betaine or 1:1 urea:betaine mixtures near their saturation levels (950 and 957 g/L. respectively) exhibit a significant antioxidant activity in oil formulation with PFs between 1.1 and 1.2 (FIG. 24). When these crude extracts are washed to yield purified rosemary extracts, the PF drastically increases to reach values ranging from 1.8 to 2.0. This means that by applying the process of the invention with the additional washing procedure, we doubled the oxidative stability of an oily formulation (here a sunflower oil).

### Example 41

### Antioxidant Activity (Rancimat Method) of the Crude and Purified Eutectic Extracts of Rosemary Obtained with Betaine:Levulinic Acid DES and Formulated in a Sunflower Oil

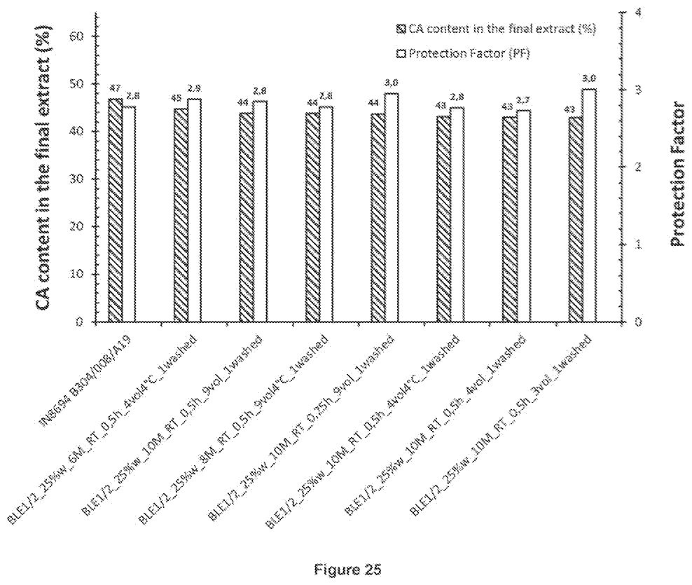

With the same Rancimat protocol as described in Example 40, we show that seven different purified rosemary extracts obtained using 1:2 betaine:levulinic acid DES exhibit a significant antioxidant activity in oil formulation with PFs between 2.7 and 3 (FIG. 25). This means that by applying the process of the invention with the additional washing procedure, we can tripled the oxidative stability of an oily formulation (here a sunflower oil). Interestingly, we shows a standard rosemary extract obtained using a traditional extraction with acetone followed by a complex purification process. The extracts of the invention are all-comparable or even better that this rosemary extract reference.

### Example 42

### Correlation between the Protection Factor Measured using the Rancimat Assay and the Carnosic Acid Content in the Final Extract (%)

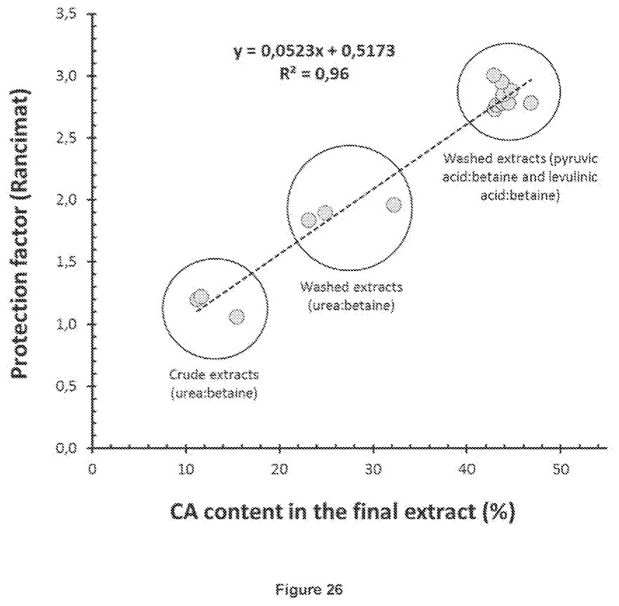

Here we plotted the the protection factor measured using the Rancimat assay and exemplified in Example 40 and 41 as a function of the carnosic acid content in the final extract (%). A linear positive correlation that can be mathematically expressed as y=0.0523x+0.5173 was found with a R² of 0.96 (FIG. 26). This means that the higher the carnosic acid content in the final extract, the higher the corresponding antioxidant activity.

### Example 43

### Effect of the Content of Salt (NaCl) in Water on the Carnosic Acid (CA) Hydrosolubility

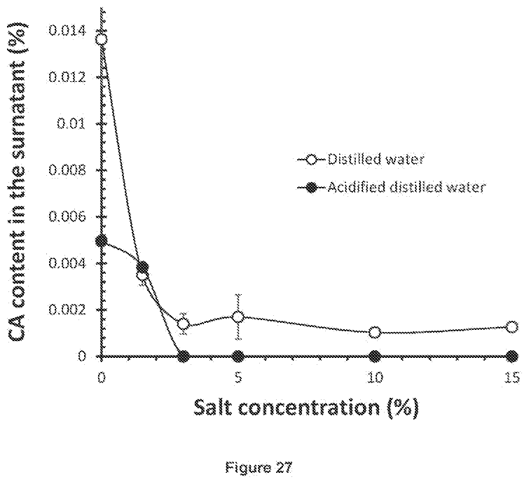

To further improve the washing procedure that leads to a high carnosic acid content in the final extract and, concomitantly, a strong antioxidant activity, here we studied the effect of the addition of sodium chloride on the CA hydrosolubility. Results shown in FIG. 27 clearly demonstrate that salt can help to reduce the amounts of CA lost during the washing procedure. Indeed, the water solubility of CA is strongly reduced by adding 3 g/L of salt both in distilled water and in acidified distilled water. Thus, the amount of CA retrieved after centrifugation following the washing procedure could be strongly improved by adding salt.

This salt addition at the washing step has been performed on an extraction of camosic acid from ground rosemary leaves for 0.5 h at room temperature and at a plant:solvent weight ratio of 10 using a 1:2 betaine:levulinic acid mixture containing 25% of water. After filtration, the carnosic acid recovery rate was found to be 79%. We then added 9 volumes of water at room temperature under magnetic stirring for 15 min and we let for settling for other 15 minutes. At the end of the procedure, we used water added with sodium chloride for the washing step (3.5%). The final carnosic acid content of the purified extract was found to be 38%, while the final mass yield was 4.5%. With this verification, we demonstrated that the addition of salt is compatible with the obtention of an extract.

Besides the washing procedure, the addition of salt can also help during the precipitation/flocculation step by using water added with 3 g/L or more of salt as an antisolvent. This could minimize the CA amounts lost during the precipitation.

### Example 44

### Extraction of Ground Sage Leaves (1 h), Cake Removal by Centrifugation then Filtration, Separation of the Extract from the Deep Eutectics Using Water as an Anti-Solvent, Recovery of the Crude Extract by Centrifugation, and Removal of Residual Deep Eutectics by a Washing Procedure with Water

A homogeneous aqueous solution of a 2:1 urea:betaine mixture was prepared near the saturation level (950 g/L). Twenty grams of sage (Salvia officinalis) leaves (containing 1.75% carnosic acid) were incubated at ambient temperature with 200 mL of this deep eutectic solution for 1 h and the enriched solution was separated from the plant by centrifugation followed by a filtration. The filtrate was analyzed by HPLC and the concentration of carnosic acid was found to be 0.67 g/L, which corresponds to a recovery rate of 28.6%. Then, the filtrate was diluted by water by a factor 10. This step of using water as an anti-solvent to precipitate the active components that will then constitute the crude extract is essential to separate most of the deep eutectics from the extract. The resulting precipitate was magnetically stirred for 15 minutes and let for settling for other 15 minutes. The precipitate was recovered by centrifugation and dried overnight at 45° C. in a vacuum oven. Finally, 573 mg of dried pellets was collected (mass yield: 2.86%), containing 7.30% of carnosic acid (HPLC quantification), which corresponds to a final recovery rate of 11.92% relative to the sage starting material.

This process was then reproduced in a separate experiment. A recovery rate of carnosic acid of 31.6% was observed in the filtrate after removal of sage residues, which is comparable to what was obtained previously. Then, an additional washing procedure using 200 mL of water was applied to remove the residual deep eutectics from the extract and further increase the carnosic acid concentration. The solvent-free eutectic extract that has been dried overnight at 45° C. in a vacuum oven final contained 14.3% of carnosic acid (HPLC quantification). The final mass yield was 1.22%, with a global recovery rate of carnosic acid of 9.95%. The extract is easily recoverable and forms a fluid powder that can be further ground.

### Example 45

### Extraction of Ground Turmeric Roots (1 h), Cake Removal by Filtration, Separation of the Extract from the Deep Eutectic Solvent Using Water as an Anti-Solvent Recovery of the Crude Extract by Centrifugation, and Removal of Residual Deep Eutectic Solvent by a Washing Procedure with Water

Homogeneous aqueous solutions of 1:1 sorbitol:levulinic acid mixture containing 20% water (SLe1/1_20% w), 1:1 proline:levulinic acid mixture containing 20% water (PLe1/1_20% w), 1:2 betaine:glycerol mixture containing 30% water (BGly1/2_30% w), 1.2 betaine:levulinic acid mixture containing 25% water (BLe1/2_25% w), and 1:2 betaine:lactic acid mixture containing 10% water (BLa1/2_10% w) were prepared. Twenty grams of ground turmenc roots (Curcuma longa) leaves (containing 2.93% curcuminoids and 1.74% curcumin) were incubated at ambient temperature or 60° C. with 200 mL of each DES (plant-solvent ratio: 1:10) for one hour and the enriched solution was separated from the plant by filtration. The filtrate was analyzed by HPLC and the recovery rate was comprised between 9 and 117% for curcuminoids and between 7 and 112% for curcumin (FIG. 27a). Then, the filtrate was diluted with four volumes of water. This step of using water as an anti-solvent to precipitate the active components that will then constitute the crude extract is essential to separate most of the DES from the extract. The resulting precipitate was magnetically stirred for 15 min and let for settling for other 15 min. The precipitate was recovered by centrifugation and then washed using 200 mL of acidic water to remove the residual DES from the extract and further increase the carnosic acid concentration. Again a centrifugation step enables to recover the pellets which constitute the solvent-free eutectic extracts. These latters have been freeze-dried overnight and finally contained between 21 and 46% of curcuminoids and between 12 and 26% for curcumin (HPLC quantification) (FIG. 27b). The final mass yield was between 0.2 and 3.9% (FIG. 27c).

### Example 46

### Chemical Composition of the Purified (Washed) Turmeric Extract Obtained in Example 45 Using a 1:2 Betaine:Lactic Acid DES

Table 3 shows some of the identified compounds found in a eutectic turmeric extract obtained using a 1:2 betaine:lactic acid containing 10% of water and described in Example 45 and FIG. 27 (sample ‘BLa1/2_10% w_10M_1h_4vol_1 washed’).

TABLE 3
Identified compounds in a eutectic turmeric purified extract

| Identified compounds in the | | |
|---|---|---|
| eutectic extract | Formula | Chemical family |
| Ar-turmerone | C₁₅H₂₁O | Sesquiterpene |
| Curcumin | C₂₁H₂₀O₆ | Curcuminoid |
| Dihydrocurcumin | C₂₁H₂₂O₆ | Curcuminoid |
| Demethoxycurcumin | C₂₀H₁₈O₅ | Curcuminoid |
| Dehydrodemethoxycurcumin | C₂₀H₂₀O₅ | Curcuminoid |
| Bisdemethoxycurcumin | C₁₉H₁₈O₄ | Curcuminoid |
| Dehydro bisdemethoxycurcumin | C₁₉H₁₈O₄ | Curcuminoid |
| Curcumadione | C₁₅H₂₃O₂ | Sesquiterpene |
| Procurcumadiol | C₁₅H₂₃O₃ | Sesquiterpene |
| Dehydrocurdione | C₁₅H₂₃O₂ | Sesquiterpene |
| Deoxy bisdemethoxycurcumin | C₁₉H₁₆O₃ | Curcuminoid |
| Deoxy dehydrobisdemethoxycurcumin | C₁₉H₁₈O₃ | Curcuminoid |
| O-Demethyldemethoxycurcumin | C₁₉H₁₆O₅ | Curcuminoid |

### Example 47

### Extraction of Ground Orange (Citrus sinensis) peel (1 h) and Cake Removal by Filtration

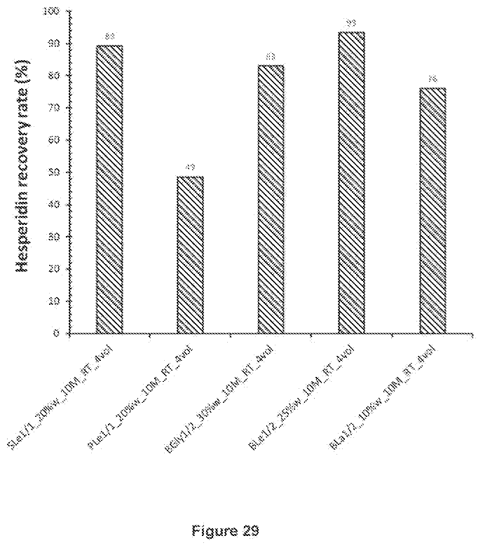

Homogeneous aqueous solutions of 1:1 sorbitol:levulinic acid mixture containing 20% water (SLe1/1_20% w), 1:1 proline:levulinic acid mixture containing 20% water (PLe1/1_20% w), 1:2 betaine:glycerol mixture containing 30% water (BGly1/2_30% w), 1:2 betaine:levulinic add mixture containing 25% water (BLe1/2_25% w), and 1:2 betaine:lactic acid mixture containing 10% water (BLa1/2_10% w) were prepared. Twenty grams of ground orange peel (containing 2.67% hesperidin) were incubated at ambient temperature with 200 mL of each DES (plant:solvent ratio: 1:10) for one hour and the enriched solution was separated from the plant by filtration. The filtrate was analyzed by HPLC and the recovery rate was comprised between 49 and 93% for hesperidin (FIG. 29).

1. A method for providing a solid biological extract comprising:
i) mixing biological material with an extraction solution comprising water and a Deep Eutectic Solvent (DES);
ii) removing any undissolved biological material from the solution obtained in (i);
iii) obtaining a flocculate and/or precipitate by adding water to and/or cooling the solution obtained in step (ii);
iv) collecting the resulting solid material obtained in step (iii) from the solution; and
v) optionally drying the solid material obtained in step (iv).

2. The method for providing the solid biological extract of claim 1:
wherein the extraction solution of step i) is free of water.

3. The method according to claim 1, wherein the solid biological extract comprises at least 2% by weight of lipophilic, hydrophobic, oil soluble and/or non-water-soluble compounds.

4. The method according to claim 3, wherein the lipophilic, hydrophobic, oil soluble and/or non-water-soluble compounds comprise one or more of phenolic compounds including phenolic acids, phenolic esters, phenolic diterpenes, flavonoids, secoiridoids, curcuminoids, bixin, capsaicinoids, cannabinoids, pyranoanthocyanins, stilbenes, phenolic alcohols, phenolic lipids, sylimarins, alkaloids, lipids, phenylpropanoids, coumarin, organic acids, terpenoids including monoterpenoids, sesquiterpenoids, diterpenoids, saponins, lignans, anthraquinone, glucosinolates, sulforaphane and isothiocyanates, triterpenoids, sapogenins or carotenoids, and mixtures thereof, from the biological material.

5. The method according to claim 1, wherein the solid biological extract comprises at least 2% by weight carnosic acid and/or its derivatives and/or other lipophilic, hydrophobic, oil soluble and/or non-water-soluble compounds present in the biological material.

6. (canceled)

7. (canceled)

8. The method according to claim 1, wherein the biological material is a plant biological material.

9. The method according to claim 8, wherein the plant biological material is obtained or obtainable from the plant roots, the aerial parts of a plant or a mixture thereof.

10. The method according to claim 8, wherein the plant biological material is obtained from or obtainable from at least one plant of the Lamiaceae species.

11. (canceled)

12. (canceled)

13. The method according to claim 1, wherein the DES is obtained by combining at least two compounds selected from methylamines, organic acids, sugars, polyols, amino acids, and urea.

14. The method according to claim 13, wherein:
a. the methylamines are selected from N-trimethylamine oxide (TMAO), betaine, glycerophosphocholine, carnitine, homarine, choline chloride, and methyl sulfonium solutes including dimethylsulfonopropionate (DMSP) and derivatives thereof, for example, their halide forms;
b. the organic acids are selected from levulinic acid, lactic acid, malic acid, maleic acid, pyruvic acid, fumaric acid, succinic acid, citric acid, citraconic acid, glutaric acid, glycolic acid, acetic acid, aconitic acid, tartaric acid, ascorbic acid, malonic acid, oxalic acid, glucuronic acid, neuraminic acid, sialic acid, shikimic acid, phytic acid, galacturonic acid, iduronic acid, hyaluronic acid, hydroxycitric acid, lactone derivatives and derivatives thereof;
c. the sugars are selected from trehalose, glucose, sucrose, lactose, ribose, fructose, galactose and derivatives thereof;
d. the polyols are selected from glycerol, erythritol, mannitol, sorbitol, xylitol, ethylene glycol, propylene glycol, ribitol, aldonitol, propanediol, inositol, pentylene glycol, and derivatives thereof; and
e. the amino acids are selected from glycine, proline, taurine, lysine, and derivatives thereof.

15. The method according to claim 1, wherein the DES is: urea and betaine, glycerol and betaine, pyruvic acid and betaine, choline chloride and urea, glycerol and choline chloride, malic acid and choline chloride, levulinic acid and betaine, lactic acid and betaine, sorbitol and levulinic acid, betaine and sorbitol, proline and levulinic acid, betaine and proline, betaine and glucose, proline and glucose, lysine and levulinic acid, glycerol and sorbitol, glycerol and lactic acid, glucose and levulinic acid, xylitol and levulinic acid, sorbitol and lactic acid, urea and betaine HCl, or glycerol and levulinic acid.

16. The method according to claim 1, wherein the concentration of the DES in the extraction solution used in step (i) is at least the minimum hydrotropic concentration (MHC) of the DES.

17. (canceled)

18. (canceled)

19. (canceled)

20. The method according to claim 1, further comprising:
vi) washing the solid obtained in step (iv) or (v) from about 1 to about 10 times or more with water and collecting the resulting solid material, and optionally drying the solid material.

21. The solid biological extract obtained or obtainable by the method as defined in claim 1.

22. (canceled)

23. The solid biological extract according to claim 21, wherein the solid biological extract comprises at about 2% or more by weight of the solid biological extract of lipophilic, hydrophobic, oil soluble and/or non-water-soluble compounds or about 10% or more by weight of the extract of lipophilic, hydrophobic, oil soluble and/or non-water-soluble compounds.

24. (canceled)

25. (canceled)

26. The solid biological extract according to claim 21, wherein the solid biological extract comprises about 2% or less by weight of the solid biological extract of DES or about 0.04% or less by weight of the solid biological extract of DES.

27. (canceled)

28. (canceled)

29. A method of utilizing the solid biological extract according to claim 21 as an anti-oxidant, anti-microbial, anti-inflammatory, as colour or pigments, as vitamins, as surfactant, as flavouring agent, as fragrance and/or as taste modifiers.

30. (canceled)

31. A nutraceutical composition, a dietary or food product for humans or animals, a nutritional supplement, a fragrance or flavouring, a pharmaceutical, a veterinary composition, or an oenological or cosmetic formulation comprising the solid biological extract according to claim 21, and optionally a pharmaceutically/veterinary acceptable ingredient.

32. The method according to claim 2, wherein the DES is obtained by combining at least two compounds selected from methylamines, organic acids, sugars, polyols, amino acids and urea.

33. The method according to claim 32, wherein:
f. the methylamines are selected from N-trimethylamine oxide (TMAO), betaine, glycerophosphocholine, carnitine, homarine, choline chloride, and methyl sulfonium solutes including dimethylsulfonopropionate (DMSP) and derivatives thereof, for example, their halide forms;
g. the organic acids are selected from levulinic acid, lactic acid, malic acid, maleic acid, pyruvic acid, fumaric acid, succinic acid, citric acid, citraconic acid, glutaric acid, glycolic acid, acetic acid, aconitic acid, tartaric acid, ascorbic acid, malonic acid, oxalic acid, glucuronic acid, neuraminic acid, sialic acid, shikimic acid, phytic acid, galacturonic acid, iduronic acid, hyaluronic acid, hydroxycitric acid, lactone derivatives and derivatives thereof;
h. the sugars are selected from trehalose, glucose, sucrose, lactose, ribose, fructose, galactose, and derivatives thereof;
i. the polyols are selected from glycerol, erythritol, mannitol, sorbitol, xylitol, ethylene glycol, propylene glycol, ribitol, aldonitol, propanediol, inositol, pentylene glycol, and derivatives thereof; and
j. the amino acids are selected from glycine, proline, taurine, lysine, and derivatives thereof;

34. The method according to claim 2, wherein the DES is: urea and betaine, glycerol and betaine, pyruvic acid and betaine, choline chloride and urea, glycerol and choline chloride, malic acid and choline chloride, levulinic acid and betaine, lactic acid and betaine, sorbitol and levulinic acid, betaine and sorbitol, proline and levulinic acid, betaine and proline, betaine and glucose, proline and glucose, lysine and levulinic acid, glycerol and sorbitol, glycerol and lactic acid, glucose and levulinic acid, xylitol and levulinic acid, sorbitol and lactic acid, urea and betaine HCl, or glycerol and levulinic acid.
